SEQUENCE LISTING (198 sequences)

SEQ ID NO: 1 - DNA, 357 bp, synthetic construct
  misc_feature 1..357: note = synthetic
  source 1..357: mol_type = other DNA
caggtgcagctggtggagtccgggggaggcgtggtccagcctgggaggtccctgagactctcctgtacagcgtctggattcaccttcagtagttatgccatgtactgggtccgccaggctccaggcaaggggctggagtgggtgtcagttatatactatgatggaagttataaatactatgcagactccgtgaagggccgattcaccatctccagagacaattccaagaacacgctgtatctgcaaatggacagcctgagagccgaggacacggctgtctattactgtgcgagttataactggaactactggtacttcgatttctggggccgtggcaccctggtcactgtctcctca

SEQ ID NO: 2 - protein, 119 aa, synthetic construct
  REGION 1..119: note = synthetic
QVQLVESGGGVVQPGRSLRLSCTASGFTFSSYAMYWVRQAPGKGLEWVSVIYYDGSYKYYADSVKGRFTISRDNSKNTLYLQMDSLRAEDTAVYYCASYNWNYWYFDFWGRGTLVTVSS

SEQ ID NO: 3 - DNA, 24 bp, synthetic construct
  misc_feature 1..24: note = synthetic
  source 1..24: mol_type = other DNA
ggattcaccttcagtagttatgcc

SEQ ID NO: 4 - protein, 8 aa, synthetic construct
  REGION 1..8: note = synthetic
GFTFSSYA

SEQ ID NO: 5 - DNA, 24 bp, synthetic construct
  misc_feature 1..24: note = synthetic
  source 1..24: mol_type = other DNA
atatactatgatggaagttataaa

SEQ ID NO: 6 - protein, 8 aa, synthetic construct
  REGION 1..8: note = synthetic
IYYDGSYK

SEQ ID NO: 7 - DNA, 36 bp, synthetic construct
  misc_feature 1..36: note = synthetic
  source 1..36: mol_type = other DNA
gcgagttataactggaactactggtacttcgatttc

SEQ ID NO: 8 - protein, 12 aa, synthetic construct
  REGION 1..12: note = synthetic
ASYNWNYWYFDF

SEQ ID NO: 9 - DNA, 324 bp, synthetic construct
  misc_feature 1..324: note = synthetic
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccgtcaaggttcagtggcagtggatctgggacagatttcactctcaccatcagcagtctgcaacctgaagattttgcaacttactactgtcaacagagttacagtacccctccgatcaccttcggccaagggacacgactggagattaaa

SEQ ID NO: 10 - protein, 108 aa, synthetic construct
  REGION 1..108: note = synthetic
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPITFGQGTRLEIK

SEQ ID NO: 11 - DNA, 18 bp, synthetic construct
  misc_feature 1..18: note = synthetic
  source 1..18: mol_type = other DNA
cagagcattagcagctat

SEQ ID NO: 12 - protein, 6 aa, synthetic construct
  REGION 1..6: note = synthetic
QSISSY

SEQ ID NO: 13-14 - skipped (2 entries)

SEQ ID NO: 15 - DNA, 30 bp, synthetic construct
  misc_feature 1..30: note = synthetic
  source 1..30: mol_type = other DNA
caacagagttacagtacccctccgatcacc

SEQ ID NO: 16 - protein, 10 aa, synthetic construct
  REGION 1..10: note = synthetic
QQSYSTPPIT

SEQ ID NO: 17 - DNA, 354 bp, synthetic construct
  misc_feature 1..354: note = synthetic
  source 1..354: mol_type = other DNA
caggtgcagctggtggagtctgggggaggcgtggtccagccgggggggtccctgagactctcctgtgcagcctctggattcaccttcagtacttatgacattcactgggtccgccaggctccaggcaaggggctggagtgggtggcagtcatatcttttgatggaagtaatagaaattatggagactccgtgaagggccgattcaccatctccagggacaattccgacaataccctgtttctggaaatgaacaatctgagatttgaagacacggctgtgtattactgtgcgaaagagggctacggtgttgacttccaacactggggccagggcaccctggtcaccgtctcctca

SEQ ID NO: 18 - protein, 118 aa, synthetic construct
  REGION 1..118: note = synthetic
QVQLVESGGGVVQPGGSLRLSCAASGFTFSTYDIHWVRQAPGKGLEWVAVISFDGSNRNYGDSVKGRFTISRDNSDNTLFLEMNNLRFEDTAVYYCAKEGYGVDFQHWGQGTLVTVSS

SEQ ID NO: 19 - DNA, 24 bp, synthetic construct
  misc_feature 1..24: note = synthetic
  source 1..24: mol_type = other DNA
ggattcaccttcagtacttatgac

SEQ ID NO: 20 - protein, 8 aa, synthetic construct
  REGION 1..8: note = synthetic
GFTFSTYD

SEQ ID NO: 21 - DNA, 24 bp, synthetic construct
  misc_feature 1..24: note = synthetic
  source 1..24: mol_type = other DNA
atatcttttgatggaagtaataga

SEQ ID NO: 22 - protein, 8 aa, synthetic construct
  REGION 1..8: note = synthetic
ISFDGSNR

SEQ ID NO: 23 - DNA, 33 bp, synthetic construct
  misc_feature 1..33: note = synthetic
  source 1..33: mol_type = other DNA
gcgaaagagggctacggtgttgacttccaacac

SEQ ID NO: 24 - protein, 11 aa, synthetic construct
  REGION 1..11: note = synthetic
AKEGYGVDFQH

SEQ ID NO: 25 - DNA, 384 bp, synthetic construct
  misc_feature 1..384: note = synthetic
  source 1..384: mol_type = other DNA
caggtgcagctgcaggagtcgggcccaggactggtgaagccttcggagaccctgtccctcacctgcactgtctctggtggctccatcagtaattactactggacctggatccggcagcccccagggaagggactggagtggattgggcatatctctaacagtgggaccaccaactataacccctccctcaagagtcgagtcaccatatcagtagacacgtccaagaaccagttctccctgaagctgacctctgtgaccgccgcagacacggccgtgtattactgtgcgagactccccctcggtattgcagcagttgatacagattattattattacttcggtatggacgtctggggccaagggaccacggtcaccgtctcctca

SEQ ID NO: 26 - protein, 128 aa, synthetic construct
  REGION 1..128: note = synthetic
QVQLQESGPGLVKPSETLSLTCTVSGGSISNYYWTWIRQPPGKGLEWIGHISNSGTTNYNPSLKSRVTISVDTSKNQFSLKLTSVTAADTAVYYCARLPLGIAAVDTDYYYYFGMDVWGQGTTVTVSS

SEQ ID NO: 27 - DNA, 24 bp, synthetic construct
  misc_feature 1..24: note = synthetic
  source 1..24: mol_type = other DNA
ggtggctccatcagtaattactac

SEQ ID NO: 28 - protein, 8 aa, synthetic construct
  REGION 1..8: note = synthetic
GGSISNYY

SEQ ID NO: 29 - DNA, 21 bp, synthetic construct
  misc_feature 1..21: note = synthetic
  source 1..21: mol_type = other DNA
atctctaacagtgggaccacc

SEQ ID NO: 30 - protein, 7 aa, synthetic construct
  REGION 1..7: note = synthetic
ISNSGTT

SEQ ID NO: 31 - DNA, 66 bp, synthetic construct
  misc_feature 1..66: note = synthetic
  source 1..66: mol_type = other DNA
gcgagactccccctcggtattgcagcagttgatacagattattattattacttcggtatggacgtc

SEQ ID NO: 32 - protein, 22 aa, synthetic construct
  REGION 1..22: note = synthetic
ARLPLGIAAVDTDYYYYFGMDV

SEQ ID NO: 33 - DNA, 348 bp, synthetic construct
  misc_feature 1..348: note = synthetic
  source 1..348: mol_type = other DNA
gaggtgcagctggtggagtctgggggaggtgtggtacggcctggggggtccctgagactctcctgtacagcctctggattcacctttgatgattatggcatgagctgggtccgccaagctccagggaagggactggagtgggtctctggtattaattggaatggtggtagaacagaatatgcagacgctgtgaagggccgattcatcatctccagagacaacgccaagaactccctgtatctgcaaatgaacagtctgagagccgaggacacggccttgtatttctgtgcgagaacggaattacggtgggactactggggccagggaaccctggtcaccgtctcctca

SEQ ID NO: 34 - protein, 116 aa, synthetic construct
  REGION 1..116: note = synthetic
EVQLVESGGGVVRPGGSLRLSCTASGFTFDDYGMSWVRQAPGKGLEWVSGINWNGGRTEYADAVKGRFIISRDNAKNSLYLQMNSLRAEDTALYFCARTELRWDYWGQGTLVTVSS

SEQ ID NO: 35 - DNA, 24 bp, synthetic construct
  misc_feature 1..24: note = synthetic
  source 1..24: mol_type = other DNA
ggattcacctttgatgattatggc

SEQ ID NO: 36 - protein, 8 aa, synthetic construct
  REGION 1..8: note = synthetic
GFTFDDYG

SEQ ID NO: 37 - DNA, 24 bp, synthetic construct
  misc_feature 1..24: note = synthetic
  source 1..24: mol_type = other DNA
attaattggaatggtggtagaaca

SEQ ID NO: 38 - protein, 8 aa, synthetic construct
  REGION 1..8: note = synthetic
INWNGGRT

SEQ ID NO: 39 - DNA, 27 bp, synthetic construct
  misc_feature 1..27: note = synthetic
  source 1..27: mol_type = other DNA
gcgagaacggaattacggtgggactac

SEQ ID NO: 40 - protein, 9 aa, synthetic construct
  REGION 1..9: note = synthetic
ARTELRWDY

SEQ ID NO: 41 - DNA, 363 bp, synthetic construct
  misc_feature 1..363: note = synthetic
  source 1..363: mol_type = other DNA
gaggtgcagctggtggagtctgggggaggcttggtacagcctggagggtccctgagactctcctgtgcagcctctggactcaccttcagtagttatgaaatgaactgggtccgccaggctccagggaaggggctggagtgggtttcacacattagtagtgctggtagtaccaaatactacgcagactctgtgaagggccgattcaccatctccagagacaacgccaagaactcactgtttctgcaaatgaacagcctgagagccgaggacacggctatttattactgtgcgggctataactacgcctactactacttcggtgtggacgtctggggccaagggaccacggtcaccgtctcctca

SEQ ID NO: 42 - protein, 121 aa, synthetic construct
  REGION 1..121: note = synthetic
EVQLVESGGGLVQPGGSLRLSCAASGLTFSSYEMNWVRQAPGKGLEWVSHISSAGSTKYYADSVKGRFTISRDNAKNSLFLQMNSLRAEDTAIYYCAGYNYAYYYFGVDVWGQGTTVTVSS

SEQ ID NO: 43 - DNA, 24 bp, synthetic construct
  misc_feature 1..24: note = synthetic
  source 1..24: mol_type = other DNA
ggactcaccttcagtagttatgaa

SEQ ID NO: 44 - protein, 8 aa, synthetic construct
  REGION 1..8: note = synthetic
GLTFSSYE

SEQ ID NO: 45 - DNA, 24 bp, synthetic construct
  misc_feature 1..24: note = synthetic
  source 1..24: mol_type = other DNA
attagtagtgctggtagtaccaaa

SEQ ID NO: 46 - protein, 8 aa, synthetic construct
  REGION 1..8: note = synthetic
ISSAGSTK

SEQ ID NO: 47 - DNA, 42 bp, synthetic construct
  misc_feature 1..42: note = synthetic
  source 1..42: mol_type = other DNA
gcgggctataactacgcctactactacttcggtgtggacgtc

SEQ ID NO: 48 - protein, 14 aa, synthetic construct
  REGION 1..14: note = synthetic
AGYNYAYYYFGVDV

SEQ ID NO: 49 - DNA, 360 bp, synthetic construct
  misc_feature 1..360: note = synthetic
  source 1..360: mol_type = other DNA
cagctgcagctgcaggagtcgggcccaggactggtgaagccttcggagaccctgtccctcatttgcactgtctctggtggctccatcagcagtaataattactactggggctggatccgccagcccccagggaaggggctggaatggattgggaatttgtatggtagtgggagtacctactacaacccgtccctcaagagtcgagtcaccatatccgtagacacgtccaagaaccagttctccctgaagctgagttttgtgaccgccgcagacacggctgtgtatcactgtgcgagatgggacactaacgggggggcttttgatatctggggccaagggacaatggtcaccgtctcttca

SEQ ID NO: 50 - protein, 120 aa, synthetic construct
  REGION 1..120: note = synthetic
QLQLQESGPGLVKPSETLSLICTVSGGSISSNNYYWGWIRQPPGKGLEWIGNLYGSGSTYYNPSLKSRVTISVDTSKNQFSLKLSFVTAADTAVYHCARWDTNGGAFDIWGQGTMVTVSS

SEQ ID NO: 51 - DNA, 30 bp, synthetic construct
  misc_feature 1..30: note = synthetic
  source 1..30: mol_type = other DNA
ggtggctccatcagcagtaataattactac

SEQ ID NO: 52 - protein, 10 aa, synthetic construct
  REGION 1..10: note = synthetic
GGSISSNNYY

SEQ ID NO: 53 - DNA, 21 bp, synthetic construct
  misc_feature 1..21: note = synthetic
  source 1..21: mol_type = other DNA
ttgtatggtagtgggagtacc

SEQ ID NO: 54 - protein, 7 aa, synthetic construct
  REGION 1..7: note = synthetic
LYGSGST

SEQ ID NO: 55 - DNA, 36 bp, synthetic construct
  misc_feature 1..36: note = synthetic
  source 1..36: mol_type = other DNA
gcgagatgggacactaacgggggggcttttgatatc

SEQ ID NO: 56 - protein, 12 aa, synthetic construct
  REGION 1..12: note = synthetic
ARWDTNGGAFDI

SEQ ID NO: 57 - DNA, 345 bp, synthetic construct
  misc_feature 1..345: note = synthetic
  source 1..345: mol_type = other DNA
caggttcagctggtgcagtctggagctgaggtgaagaagcctggggcctcagtgaaggtctcctgcaaggcttctggttacacctttaccagccttggaatcagctgggtgcgacaggcccctggacaagggcttgagtggatgggatggatcagcgcctacagtggtaatagagactatgcacagaagttccagggcagagtcaccatgaccacagacacatccacgagcacagcctatatggagctgaggagcctgagatctgacgacacggccgtgtattattgtgcgagatctctacgttttgactactggggccagggaaccctggtcaccgtctcctca

SEQ ID NO: 58 - protein, 115 aa, synthetic construct
  REGION 1..115: note = synthetic
QVQLVQSGAEVKKPGASVKVSCKASGYTFTSLGISWVRQAPGQGLEWMGWISAYSGNRDYAQKFQGRVTMTTDTSTSTAYMELRSLRSDDTAVYYCARSLRFDYWGQGTLVTVSS

SEQ ID NO: 59 - DNA, 24 bp, synthetic construct
  misc_feature 1..24: note = synthetic
  source 1..24: mol_type = other DNA
ggttacacctttaccagccttgga

SEQ ID NO: 60 - protein, 8 aa, synthetic construct
  REGION 1..8: note = synthetic
GYTFTSLG

SEQ ID NO: 61 - DNA, 24 bp, synthetic construct
  misc_feature 1..24: note = synthetic
  source 1..24: mol_type = other DNA
atcagcgcctacagtggtaataga

SEQ ID NO: 62 - protein, 8 aa, synthetic construct
  REGION 1..8: note = synthetic
ISAYSGNR

SEQ ID NO: 63 - DNA, 24 bp, synthetic construct
  misc_feature 1..24: note = synthetic
  source 1..24: mol_type = other DNA
gcgagatctctacgttttgactac

SEQ ID NO: 64 - protein, 8 aa, synthetic construct
  REGION 1..8: note = synthetic
ARSLRFDY

SEQ ID NO: 65 - DNA, 357 bp, synthetic construct
  misc_feature 1..357: note = synthetic
  source 1..357: mol_type = other DNA
gaggtgcagctggtggagtctgggggaggcttggtacagcctggcaggtccctgagactctcctgtgcagcctcaggattcaccttggatgattttgccatgcactgggtccggcaagctccagggaagggcctggagtgggtctcaggtattagttggaatagcggtaatataggttatgcggactctgtgaagggccgcttcaccatctccagagacaacgccaggaactccctgtatctgcaaatgaacagtctgagagcggaagacacggccttgtattactgcacctcgggatactacctctactacgctatggacgtctggggccaggggaccacggtcaccgtctcctca

SEQ ID NO: 66 - protein, 119 aa, synthetic construct
  REGION 1..119: note = synthetic
EVQLVESGGGLVQPGRSLRLSCAASGFTLDDFAMHWVRQAPGKGLEWVSGISWNSGNIGYADSVKGRFTISRDNARNSLYLQMNSLRAEDTALYYCTSGYYLYYAMDVWGQGTTVTVSS

SEQ ID NO: 67 - DNA, 24 bp, synthetic construct
  misc_feature 1..24: note = synthetic
  source 1..24: mol_type = other DNA
ggattcaccttggatgattttgcc

SEQ ID NO: 68 - protein, 8 aa, synthetic construct
  REGION 1..8: note = synthetic
GFTLDDFA

SEQ ID NO: 69 - DNA, 24 bp, synthetic construct
  misc_feature 1..24: note = synthetic
  source 1..24: mol_type = other DNA
attagttggaatagcggtaatata

SEQ ID NO: 70 - protein, 8 aa, synthetic construct
  REGION 1..8: note = synthetic
ISWNSGNI

SEQ ID NO: 71 - DNA, 36 bp, synthetic construct
  misc_feature 1..36: note = synthetic
  source 1..36: mol_type = other DNA
acctcgggatactacctctactacgctatggacgtc

SEQ ID NO: 72 - protein, 12 aa, synthetic construct
  REGION 1..12: note = synthetic
TSGYYLYYAMDV

SEQ ID NO: 73 - DNA, 357 bp, synthetic construct
  misc_feature 1..357: note = synthetic
  source 1..357: mol_type = other DNA
caggtgcagctggtgcagtctgggcctgaggtgaagaagcctgggtcttcggtgaaggtctcctgcaaggcttctggaggcaccatcgacagaaatgaaatcacctgggtgcgacaggcccctggacaagggcttgagtggatgggagtaatcatccctatcttaggtgaggcacactacgaacgaaaattccagggcagactcacgattaccatggacgagtccacgagtacagcctatatggaactgagtaatctgagatctgaggacacggccgtatattactgtgcgagagagagggcagctcgaggctaccttgactactggggccagggaaccctggtcaccgtctcctca

SEQ ID NO: 74 - protein, 119 aa, synthetic construct
  REGION 1..119: note = synthetic
QVQLVQSGPEVKKPGSSVKVSCKASGGTIDRNEITWVRQAPGQGLEWMGVIIPILGEAHYERKFQGRLTITMDESTSTAYMELSNLRSEDTAVYYCARERAARGYLDYWGQGTLVTVSS

SEQ ID NO: 75 - DNA, 24 bp, synthetic construct
  misc_feature 1..24: note = synthetic
  source 1..24: mol_type = other DNA
ggaggcaccatcgacagaaatgaa

SEQ ID NO: 76 - protein, 8 aa, synthetic construct
  REGION 1..8: note = synthetic
GGTIDRNE

SEQ ID NO: 77 - DNA, 24 bp, synthetic construct
  misc_feature 1..24: note = synthetic
  source 1..24: mol_type = other DNA
atcatccctatcttaggtgaggca

SEQ ID NO: 78 - protein, 8 aa, synthetic construct
  REGION 1..8: note = synthetic
IIPILGEA

SEQ ID NO: 79 - DNA, 36 bp, synthetic construct
  misc_feature 1..36: note = synthetic
  source 1..36: mol_type = other DNA
gcgagagagagggcagctcgaggctaccttgactac

SEQ ID NO: 80 - protein, 12 aa, synthetic construct
  REGION 1..12: note = synthetic
ARERAARGYLDY

SEQ ID NO: 81 - DNA, 360 bp, synthetic construct
  misc_feature 1..360: note = synthetic
  source 1..360: mol_type = other DNA
gaggtgcagctggtggagtctgggggaggcttggtccagccgggggggtccctgagactctcctgtacagcctctggattcaccttcagtaattatgctatattctgggtccgccaggctccagggaagggactggaatatgtttcaggtattagtaataatggggttactaaatattatgcagactctgtgagggacagattcaccatctccagagacaattccaggaacacggtgtctattcaaatggacagcctgagaactgaggacatggctgtgtattactgtgcgcgagaaaactggggatcggacgatggttttgatatctggggccaaggaacaatggtcaccgtctcttca

SEQ ID NO: 82 - protein, 120 aa, synthetic construct
  REGION 1..120: note = synthetic
EVQLVESGGGLVQPGGSLRLSCTASGFTFSNYAIFWVRQAPGKGLEYVSGISNNGVTKYYADSVRDRFTISRDNSRNTVSIQMDSLRTEDMAVYYCARENWGSDDGFDIWGQGTMVTVSS

SEQ ID NO: 83 - DNA, 24 bp, synthetic construct
  misc_feature 1..24: note = synthetic
  source 1..24: mol_type = other DNA
ggattcaccttcagtaattatgct

SEQ ID NO: 84 - protein, 8 aa, synthetic construct
  REGION 1..8: note = synthetic
GFTFSNYA

SEQ ID NO: 85 - DNA, 24 bp, synthetic construct
  misc_feature 1..24: note = synthetic
  source 1..24: mol_type = other DNA
attagtaataatggggttactaaa

SEQ ID NO: 86 - protein, 8 aa, synthetic construct
  REGION 1..8: note = synthetic
ISNNGVTK

SEQ ID NO: 87 - DNA, 39 bp, synthetic construct
  misc_feature 1..39: note = synthetic
  source 1..39: mol_type = other DNA
gcgcgagaaaactggggatcggacgatggttttgatatc

SEQ ID NO: 88 - protein, 13 aa, synthetic construct
  REGION 1..13: note = synthetic
ARENWGSDDGFDI

SEQ ID NO: 89 - DNA, 357 bp, synthetic construct
  misc_feature 1..357: note = synthetic
  source 1..357: mol_type = other DNA
caggtgcagttggtgcagtctgggactgaggtgaagaagcctgggtcctcggtgaaggtctcctgcaaggcttctggaggcaccttcagcacctatcctatcaattgggtgcgacaggcccctggacaagggcttgagtggatgggagggatcatccctatctttggtacggcaagctacgcacagaagttccagggcagagtcgcgattaccacggacgaatccacgagcacagcctacatggagctgaacagcctgagatctgaggacacggccgtgtattattgtgcgagagagaggccgaactggggatctaatgactactggggccagggaaccctggtcaccgtctcctca

SEQ ID NO: 90 - protein, 119 aa, synthetic construct
  REGION 1..119: note = synthetic
QVQLVQSGTEVKKPGSSVKVSCKASGGTFSTYPINWVRQAPGQGLEWMGGIIPIFGTASYAQKFQGRVAITTDESTSTAYMELNSLRSEDTAVYYCARERPNWGSNDYWGQGTLVTVSS

SEQ ID NO: 91 - DNA, 24 bp, synthetic construct
  misc_feature 1..24: note = synthetic
  source 1..24: mol_type = other DNA
ggaggcaccttcagcacctatcct

SEQ ID NO: 92 - protein, 8 aa, synthetic construct
  REGION 1..8: note = synthetic
GGTFSTYP

SEQ ID NO: 93 - DNA, 24 bp, synthetic construct
  misc_feature 1..24: note = synthetic
  source 1..24: mol_type = other DNA
atcatccctatctttggtacggca

SEQ ID NO: 94 - protein, 8 aa, synthetic construct
  REGION 1..8: note = synthetic
IIPIFGTA

SEQ ID NO: 95 - DNA, 36 bp, synthetic construct
  misc_feature 1..36: note = synthetic
  source 1..36: mol_type = other DNA
gcgagagagaggccgaactggggatctaatgactac

SEQ ID NO: 96 - protein, 12 aa, synthetic construct
  REGION 1..12: note = synthetic
ARERPNWGSNDY

SEQ ID NO: 97 - DNA, 366 bp, synthetic construct
  misc_feature 1..366: note = synthetic
  source 1..366: mol_type = other DNA
caggtgcagctggtggagtctgggggaggcgtggtccagcctgggaggtccctgagactcgcctgtgcagcgtctggattcacctttagaagttatggcatgcactgggtccgccaggctccaggcaaggggctggagtgggtggcagttatctggtatgatggaagtaataaatactatacagactccgtgaagggccgattcaccatctccagagacaattccaagaacacgctgtatctgcaaatgaacagcctgagagccgaggacacggctatgtattactgtgcgatagacaaatgggctacggtgactacgtacctgtttgactactggggccagggaaccctggtcaccgtctcctca

SEQ ID NO: 98 - protein, 122 aa, synthetic construct
  REGION 1..122: note = synthetic
QVQLVESGGGVVQPGRSLRLACAASGFTFRSYGMHWVRQAPGKGLEWVAVIWYDGSNKYYTDSVKGRFTISRDNSKNTLYLQMNSLRAEDTAMYYCAIDKWATVTTYLFDYWGQGTLVTVSS

SEQ ID NO: 99 - DNA, 24 bp, synthetic construct
  misc_feature 1..24: note = synthetic
  source 1..24: mol_type = other DNA
ggattcacctttagaagttatggc

SEQ ID NO: 100 - protein, 8 aa, synthetic construct
  REGION 1..8: note = synthetic
GFTFRSYG

SEQ ID NO: 101 - DNA, 24 bp, synthetic construct
  misc_feature 1..24: note = synthetic
  source 1..24: mol_type = other DNA
atctggtatgatggaagtaataaa

SEQ ID NO: 102 - protein, 8 aa, synthetic construct
  REGION 1..8: note = synthetic
IWYDGSNK

SEQ ID NO: 103 - DNA, 45 bp, synthetic construct
  misc_feature 1..45: note = synthetic
  source 1..45: mol_type = other DNA
gcgatagacaaatgggctacggtgactacgtacctgtttgactac

SEQ ID NO: 104 - protein, 15 aa, synthetic construct
  REGION 1..15: note = synthetic
AIDKWATVTTYLFDY

SEQ ID NO: 105 - DNA, 363 bp, synthetic construct
  misc_feature 1..363: note = synthetic
  source 1..363: mol_type = other DNA
gaggtgcagctggtggagtctgggggaggcttggtacagtctggagggtccctgagactctcttgtgcagcctctggattcaccttcagtcgttatgaaatgaactgggtccgccaggctccagggaaggggctggagtgggttgcatatattagtaatactggtactaccagggactattcagactctgtgaagggccgattcaccatctccagagacaacgccaagaactcactatatctacaaatgagcagcctgagagtcgaggacacggctgtttattattgtgtgagagagaaatttagtgggacctctgactggttcgacccctggggccagggaaccctggtcaccgtctcctca

SEQ ID NO: 106 - protein, 121 aa, synthetic construct
  REGION 1..121: note = synthetic
EVQLVESGGGLVQSGGSLRLSCAASGFTFSRYEMNWVRQAPGKGLEWVAYISNTGTTRDYSDSVKGRFTISRDNAKNSLYLQMSSLRVEDTAVYYCVREKFSGTSDWFDPWGQGTLVTVSS

SEQ ID NO: 107 - DNA, 24 bp, synthetic construct
  misc_feature 1..24: note = synthetic
  source 1..24: mol_type = other DNA
ggattcaccttcagtcgttatgaa

SEQ ID NO: 108 - protein, 8 aa, synthetic construct
  REGION 1..8: note = synthetic
GFTFSRYE

SEQ ID NO: 109 - DNA, 24 bp, synthetic construct
  misc_feature 1..24: note = synthetic
  source 1..24: mol_type = other DNA
attagtaatactggtactaccagg

SEQ ID NO: 110 - protein, 8 aa, synthetic construct
  REGION 1..8: note = synthetic
ISNTGTTR

SEQ ID NO: 111 - DNA, 42 bp, synthetic construct
  misc_feature 1..42: note = synthetic
  source 1..42: mol_type = other DNA
gtgagagagaaatttagtgggacctctgactggttcgacccc

SEQ ID NO: 112 - protein, 14 aa, synthetic construct
  REGION 1..14: note = synthetic
VREKFSGTSDWFDP

SEQ ID NO: 113 - DNA, 375 bp, synthetic construct
  misc_feature 1..375: note = synthetic
  source 1..375: mol_type = other DNA
gaggtgcagctggtggagtctgggggaggcttggtgcagcctggggggtccctgagactctcctgtgcagcctctggattcacctttagtagctatgccatgaactgggtccgccaggctccagggaaggggctggaatgggtctcagctattagtggtagtggtgatagcacatactacacagactccgtgaagggccggttcaccatctccagagacaattccaagaacacgctgtatctgcaaatgaacaccctgagagccgaggacacggccgtatattactgtgcgaaagaggagattgttatgttggtaggtgctaccccggggtggttcgacccctggggccagggaaccctggtcaccgtctcctca

SEQ ID NO: 114 - protein, 125 aa, synthetic construct
  REGION 1..125: note = synthetic
EVQLVESGGGLVQPGGSLRLSCAASGFTFSSYAMNWVRQAPGKGLEWVSAISGSGDSTYYTDSVKGRFTISRDNSKNTLYLQMNTLRAEDTAVYYCAKEEIVMLVGATPGWFDPWGQGTLVTVSS

SEQ ID NO: 115 - DNA, 24 bp, synthetic construct
  misc_feature 1..24: note = synthetic
  source 1..24: mol_type = other DNA
ggattcacctttagtagctatgcc

SEQ ID NO: 116 - protein, 8 aa, synthetic construct
  REGION 1..8: note = synthetic
GFTFSSYA

SEQ ID NO: 117 - DNA, 24 bp, synthetic construct
  misc_feature 1..24: note = synthetic
  source 1..24: mol_type = other DNA
attagtggtagtggtgatagcaca

SEQ ID NO: 118 - protein, 8 aa, synthetic construct
  REGION 1..8: note = synthetic
ISGSGDST

SEQ ID NO: 119 - DNA, 54 bp, synthetic construct
  misc_feature 1..54: note = synthetic
  source 1..54: mol_type = other DNA
gcgaaagaggagattgttatgttggtaggtgctaccccggggtggttcgacccc

SEQ ID NO: 120 - protein, 18 aa, synthetic construct
  REGION 1..18: note = synthetic
AKEEIVMLVGATPGWFDP

SEQ ID NO: 121 - DNA, 387 bp, synthetic construct
  misc_feature 1..387: note = synthetic
  source 1..387: mol_type = other DNA
gaggtgcagctggtgcagtctggagcagaggtgaaaaagcccggggagtctctgaagatctcctgtaagggttctggatacagttttaccagttactggatcgactgggtgcgccagatgcccgggaaaggcctggagtggatggggatcatctatcctgatgactctgataccagatacagtccgtccttccaaggccaggtcaccatgtcagtcgacaagtccatcagcaccgcctacctgcagtggagcagcctgaaggcctcggacaccgccatatattactgtgcgagacatgagagaggtatagtagaagctggtacggattactactactactacggtatggacgtctggggccaagggaccacggtcaccgtctcctca

SEQ ID NO: 122 - protein, 129 aa, synthetic construct
  REGION 1..129: note = synthetic
EVQLVQSGAEVKKPGESLKISCKGSGYSFTSYWIDWVRQMPGKGLEWMGIIYPDDSDTRYSPSFQGQVTMSVDKSISTAYLQWSSLKASDTAIYYCARHERGIVEAGTDYYYYYGMDVWGQGTTVTVSS

SEQ ID NO: 123 - DNA, 24 bp, synthetic construct
  misc_feature 1..24: note = synthetic
  source 1..24: mol_type = other DNA
ggatacagttttaccagttactgg

SEQ ID NO: 124 - protein, 8 aa, synthetic construct
  REGION 1..8: note = synthetic
GYSFTSYW

SEQ ID NO: 125 - DNA, 24 bp, synthetic construct
  misc_feature 1..24: note = synthetic
  source 1..24: mol_type = other DNA
atctatcctgatgactctgatacc

SEQ ID NO: 126 - protein, 8 aa, synthetic construct
  REGION 1..8: note = synthetic
IYPDDSDT

SEQ ID NO: 127 - DNA, 66 bp, synthetic construct
  misc_feature 1..66: note = synthetic
  source 1..66: mol_type = other DNA
gcgagacatgagagaggtatagtagaagctggtacggattactactactactacggtatggacgtc

SEQ ID NO: 128 - protein, 22 aa, synthetic construct
  REGION 1..22: note = synthetic
ARHERGIVEAGTDYYYYYGMDV

SEQ ID NO: 129 - DNA, 357 bp, synthetic construct
  misc_feature 1..357: note = synthetic
  source 1..357: mol_type = other DNA
caggttcagctggtgcagtctggagctgaggtgatgaagcctggggcctcagtgaaggtctcctgcagggcttctggttacacctttagcagctatgatatcaactgggtgcgacaggcccctggacaaggacttgagtggatgggatggatcagcactgccactggtaacacaaactatccacagaaggtccagggcagagtcaccatgaccacagacacatcaacgaacacagcctacatggagctgaggagcctgagatctgacgacacggccgtatattattgtacgagagagggcaattcgaagggctggctcgacccctggggccagggaaccctggtcaccgtctcctca

SEQ ID NO: 130 - protein, 119 aa, synthetic construct
  REGION 1..119: note = synthetic
QVQLVQSGAEVMKPGASVKVSCRASGYTFSSYDINWVRQAPGQGLEWMGWISTATGNTNYPQKVQGRVTMTTDTSTNTAYMELRSLRSDDTAVYYCTREGNSKGWLDPWGQGTLVTVSS

SEQ ID NO: 131 - DNA, 24 bp, synthetic construct
  misc_feature 1..24: note = synthetic
  source 1..24: mol_type = other DNA
ggttacacctttagcagctatgat

SEQ ID NO: 132 - protein, 8 aa, synthetic construct
  REGION 1..8: note = synthetic
GYTFSSYD

SEQ ID NO: 133 - DNA, 24 bp, synthetic construct
  misc_feature 1..24: note = synthetic
  source 1..24: mol_type = other DNA
atcagcactgccactggtaacaca

SEQ ID NO: 134 - protein, 8 aa, synthetic construct
  REGION 1..8: note = synthetic
ISTATGNT

SEQ ID NO: 135 - DNA, 36 bp, synthetic construct
  misc_feature 1..36: note = synthetic
  source 1..36: mol_type = other DNA
acgagagagggcaattcgaagggctggctcgacccc

SEQ ID NO: 136 - protein, 12 aa, synthetic construct
  REGION 1..12: note = synthetic
TREGNSKGWLDP

SEQ ID NO: 137 - DNA, 363 bp, synthetic construct
  misc_feature 1..363: note = synthetic
  source 1..363: mol_type = other DNA
caggtacagctgcagcagtcaggtcccggacaaatgaagtcctcgcagaccctcacactcacctgtgccatctccggggacagtgtctctagcaacagtgctatttggtactggatcaggcagtccccatcgagaggccttgagtggctggggaggacaaagttcaggtccaaatggtataatgattttgcactatctatgaaaagtcgaataaccatcaacccagacacatccaagaaccagttctccctgcacctgaactctgtgactcccgaggacacggctgtatattactgtgcaagatataagtgggaactttactttgactactggggccagggaaccctggtcaccgtctcctca

SEQ ID NO: 138 - protein, 121 aa, synthetic construct
  REGION 1..121: note = synthetic
QVQLQQSGPGQMKSSQTLTLTCAISGDSVSSNSAIWYWIRQSPSRGLEWLGRTKFRSKWYNDFALSMKSRITINPDTSKNQFSLHLNSVTPEDTAVYYCARYKWELYFDYWGQGTLVTVSS

SEQ ID NO: 139 - DNA, 30 bp, synthetic construct
  misc_feature 1..30: note = synthetic
  source 1..30: mol_type = other DNA
ggggacagtgtctctagcaacagtgctatt

SEQ ID NO: 140 - protein, 10 aa, synthetic construct
  REGION 1..10: note = synthetic
GDSVSSNSAI

SEQ ID NO: 141 - DNA, 27 bp, synthetic construct
  misc_feature 1..27: note = synthetic
  source 1..27: mol_type = other DNA
acaaagttcaggtccaaatggtataat

SEQ ID NO: 142 - protein, 9 aa, synthetic construct
  REGION 1..9: note = synthetic
TKFRSKWYN

SEQ ID NO: 143 - DNA, 33 bp, synthetic construct
  misc_feature 1..33: note = synthetic
  source 1..33: mol_type = other DNA
gcaagatataagtgggaactttactttgactac

SEQ ID NO: 144 - protein, 11 aa, synthetic construct
  REGION 1..11: note = synthetic
ARYKWELYFDY

SEQ ID NO: 145 - DNA, 354 bp, synthetic construct
  misc_feature 1..354: note = synthetic
  source 1..354: mol_type = other DNA
gaggtgcagctggtggagtctgggggaggcttggtccagcctggggggtccctgagactctcctgtgcagcctctggattcacctttagtacctattggatgaactgggtccgccaggctccagggaaggggctggagtgggtggccaacataaaggaagatggaagtgagaaatactatgtggactctgtgaagggccgattcaccatctccagagacaacgccaagaactcactgtatctgcaaatgaacagcctgagagccgaggacacggctgtgtattactgtgcgagagagggttttggagtggttacctatatctggggccaagggacaatggtcaccgtctcttca

SEQ ID NO: 146 - protein, 118 aa, synthetic construct
  REGION 1..118: note = synthetic
EVQLVESGGGLVQPGGSLRLSCAASGFTFSTYWMNWVRQAPGKGLEWVANIKEDGSEKYYVDSVKGRFTISRDNAKNSLYLQMNSLRAEDTAVYYCAREGFGVVTYIWGQGTMVTVSS

SEQ ID NO: 147 - DNA, 24 bp, synthetic construct
  misc_feature 1..24: note = synthetic
  source 1..24: mol_type = other DNA
ggattcacctttagtacctattgg

SEQ ID NO: 148 - protein, 8 aa, synthetic construct
  REGION 1..8: note = synthetic
GFTFSTYW

SEQ ID NO: 149 - DNA, 24 bp, synthetic construct
  misc_feature 1..24: note = synthetic
  source 1..24: mol_type = other DNA
ataaaggaagatggaagtgagaaa

SEQ ID NO: 150 - protein, 8 aa, synthetic construct
  REGION 1..8: note = synthetic
IKEDGSEK

SEQ ID NO: 151 - DNA, 33 bp, synthetic construct
  misc_feature 1..33: note = synthetic
  source 1..33: mol_type = other DNA
gcgagagagggttttggagtggttacctatatc

SEQ ID NO: 152 - protein, 11 aa, synthetic construct
  REGION 1..11: note = synthetic
AREGFGVVTYI

SEQ ID NO: 153 - DNA, 351 bp, synthetic construct
  misc_feature 1..351: note = synthetic
  source 1..351: mol_type = other DNA
caggtgcagctggtggagtctgggggaggcttggtcaagcctggagggtccctgagactctcctgtgcagcctctggattcaccttcagtgactactatatgacctggatccgccagactccagggaaggggctggattgggtttcatacattagttctagtggtactaacaaatacaacgcagactctgtgaagggccgattcaccatctccagggacaacgccaagaactcactgtatctgcaaatgaacagcctgcgagccgaggacacggccgtatattactgtgtgagagaccccccctggggtatggacgtctggggccaagggaccacggtcaccgtctcctca

SEQ ID NO: 154 - protein, 117 aa, synthetic construct
  REGION 1..117: note = synthetic
QVQLVESGGGLVKPGGSLRLSCAASGFTFSDYYMTWIRQTPGKGLDWVSYISSSGTNKYNADSVKGRFTISRDNAKNSLYLQMNSLRAEDTAVYYCVRDPPWGMDVWGQGTTVTVSS

SEQ ID NO: 155 - DNA, 24 bp, synthetic construct
  misc_feature 1..24: note = synthetic
  source 1..24: mol_type = other DNA
ggattcaccttcagtgactactat

SEQ ID NO: 156 - protein, 8 aa, synthetic construct
  REGION 1..8: note = synthetic
GFTFSDYY

SEQ ID NO: 157 - DNA, 24 bp, synthetic construct
  misc_feature 1..24: note = synthetic
  source 1..24: mol_type = other DNA
attagttctagtggtactaacaaa

SEQ ID NO: 158 - protein, 8 aa, synthetic construct
  REGION 1..8: note = synthetic
ISSSGTNK

SEQ ID NO: 159 - DNA, 30 bp, synthetic construct
  misc_feature 1..30: note = synthetic
  source 1..30: mol_type = other DNA
gtgagagaccccccctggggtatggacgtc

SEQ ID NO: 160 - protein, 10 aa, synthetic construct
  REGION 1..10: note = synthetic
VRDPPWGMDV

SEQ ID NO: 161 - DNA, 360 bp, synthetic construct
  misc_feature 1..360: note = synthetic
  source 1..360: mol_type = other DNA
gaggtgcagctggtggagtctgggggaggcttggtacagcctggggggtccctgagactctcctgtgttgcctctggattcaccttcaataaatacgacatgcactgggtccgccaaactactggaaaaggtctagagtgggtctcaggtattgatactgatggtgacacatactatccaggctccgtgaagggccgattcaccatctccagagaaaatgccgagaactccctgtatcttcaaatgaacggcctgagagtcggggacacggctgtgtattactgtgcaagatggccttggagtggtttctatggtgcttttgatatctggggccaagggacaatggtcaccgtctcttca

SEQ ID NO: 162 - protein, 120 aa, synthetic construct
  REGION 1..120: note = synthetic
EVQLVESGGGLVQPGGSLRLSCVASGFTFNKYDMHWVRQTTGKGLEWVSGIDTDGDTYYPGSVKGRFTISRENAENSLYLQMNGLRVGDTAVYYCARWPWSGFYGAFDIWGQGTMVTVSS

SEQ ID NO: 163 - DNA, 24 bp, synthetic construct
  misc_feature 1..24: note = synthetic
  source 1..24: mol_type = other DNA
ggattcaccttcaataaatacgac

SEQ ID NO: 164 - protein, 8 aa, synthetic construct
  REGION 1..8: note = synthetic
GFTFNKYD

SEQ ID NO: 165 - DNA, 21 bp, synthetic construct
  misc_feature 1..21: note = synthetic
  source 1..21: mol_type = other DNA
attgatactgatggtgacaca

SEQ ID NO: 166 - protein, 7 aa, synthetic construct
  REGION 1..7: note = synthetic
IDTDGDT

SEQ ID NO: 167 - DNA, 42 bp, synthetic construct
  misc_feature 1..42: note = synthetic
  source 1..42: mol_type = other DNA
gcaagatggccttggagtggtttctatggtgcttttgatatc

SEQ ID NO: 168 - protein, 14 aa, synthetic construct
  REGION 1..14: note = synthetic
ARWPWSGFYGAFDI

SEQ ID NO: 169 - DNA, 372 bp, synthetic construct
  misc_feature 1..372: note = synthetic
  source 1..372: mol_type = other DNA
caggtgcagctgcaggagtcgggcccaggactggtgaagccttcacagaccctgtccctcacctgcactgtctctggtggctccatcagcagcggtggtgactactggagctggatccgccagctcccagggaagggcctggagtggattgggtacatctattacagtgggagcgcctactataatccgtccctcaagagtcgaggtaccatatcaatagacacgtctaagaaccagttctccctgaagctgacctctgtgactgccgcggacacggccgtatatttctgtgtgaaattacgatttttggagtggttcttggggggctggttcggcccctggggccagggaaccctggtcaccgtctcctca

SEQ ID NO: 170 - protein, 124 aa, synthetic construct
  REGION 1..124: note = synthetic
QVQLQESGPGLVKPSQTLSLTCTVSGGSISSGGDYWSWIRQLPGKGLEWIGYIYYSGSAYYNPSLKSRGTISIDTSKNQFSLKLTSVTAADTAVYFCVKLRFLEWFLGGWFGPWGQGTLVTVSS

SEQ ID NO: 171 - DNA, 30 bp, synthetic construct
  misc_feature 1..30: note = synthetic
  source 1..30: mol_type = other DNA
ggtggctccatcagcagcggtggtgactac

SEQ ID NO: 172 - protein, 10 aa, synthetic construct
  REGION 1..10: note = synthetic
GGSISSGGDY

SEQ ID NO: 173 - DNA, 21 bp, synthetic construct
  misc_feature 1..21: note = synthetic
  source 1..21: mol_type = other DNA
atctattacagtgggagcgcc

SEQ ID NO: 174 - protein, 7 aa, synthetic construct
  REGION 1..7: note = synthetic
IYYSGSA

SEQ ID NO: 175 - DNA, 48 bp, synthetic construct
  misc_feature 1..48: note = synthetic
  source 1..48: mol_type = other DNA
gtgaaattacgatttttggagtggttcttggggggctggttcggcccc

SEQ ID NO: 176 - protein, 16 aa, synthetic construct
  REGION 1..16: note = synthetic
VKLRFLEWFLGGWFGP

SEQ ID NO: 177 - DNA, 351 bp, synthetic construct
  misc_feature 1..351: note = synthetic
  source 1..351: mol_type = other DNA
gaggtgcagctggtggagtctgggggaggcttggtacagccgggggggtccctgagactctcctgtgcagcctctggattcacctttagcaactatggcatgacctgggtccgccaggctccagggaagggcctggaatgggtctcagctattactggtggtggtggtagcacatactactcaaactccgtgaagggccggttcaccatctccagagacaattccaagaacacggtgtatctgcgaatgaacagtgtgagagccgaggacacggccgtatattactgtgcgaaatataagtggaacttcgtggacgactggggccagggaaccacggtcaccgtctcctca

SEQ ID NO: 178 - protein, 117 aa, synthetic construct
  REGION 1..117: note = synthetic
EVQLVESGGGLVQPGGSLRLSCAASGFTFSNYGMTWVRQAPGKGLEWVSAITGGGGSTYYSNSVKGRFTISRDNSKNTVYLRMNSVRAEDTAVYYCAKYKWNFVDDWGQGTTVTVSS

SEQ ID NO: 179 - DNA, 24 bp, synthetic construct
  misc_feature 1..24: note = synthetic
  source 1..24: mol_type = other DNA
ggattcacctttagcaactatggc

SEQ ID NO: 180 - protein, 8 aa, synthetic construct
  REGION 1..8: note = synthetic
GFTFSNYG

SEQ ID NO: 181 - DNA, 24 bp, synthetic construct
  misc_feature 1..24: note = synthetic
  source 1..24: mol_type = other DNA
attactggtggtggtggtagcaca

SEQ ID NO: 182 - protein, 8 aa, synthetic construct
  REGION 1..8: note = synthetic
ITGGGGST

SEQ ID NO: 183 - DNA, 30 bp, synthetic construct
  misc_feature 1..30: note = synthetic
  source 1..30: mol_type = other DNA
gcgaaatataagtggaacttcgtggacgac

SEQ ID NO: 184 - protein, 10 aa, synthetic construct
  REGION 1..10: note = synthetic
AKYKWNFVDD

SEQ ID NO: 185 - protein, 918 aa, synthetic construct
  REGION 1..918: note = human GP130 UniProtKB - Q17RA0
MLTLQTWLVQALFIFLTTESTGELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEAIVVPVCLAFLLTTLLGVLFCFNKRDLIKKHIWPNVPDPSKSHIAQWSPHTPPRHNFNSKDQMYSDGNFTDVSVVEIEANDKKPFPEDLKSLDLFKKEKINTEGHSSGIGGSSCMSSSRPSISSSDENESSQNTSSTVQYSTVVHSGYRHQVPSVQVFSRSESTQPLLDSEERPEDLQLVDHVDGGDGILPRQQYFKQNCSQHESSPDISHFERSKQVSSVNEEDFVRLKQQISDHISQSCGSGQMKMFQEVSAADAFGPGTEGQVERFETVGMEAATDEGMPKSYLPQTVRQGGYMPQ

SEQ ID NO: 186 - protein, 1165 aa, synthetic construct
  REGION 1..1165: note = human LEPR (UniProtKB/Swiss-Prot Accession No. P48357)
MICQKFCVVLLHWEFIYVITAFNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDAGLYVIVPVIISSSILLLGTLLISHQRMKKLFWEDVPNPKNCSWAQGLNFQKPETFEHLFIKHTASVTCGPLLLEPETISEDISVDTSWKNKDEMMPTTVVSLLSTTDLEKGSVCISDQFNSVNFSEAEGTEVTYEDESQRQPFVKYATLISNSKPSETGEEQGLINSSVTKCFSSKNSPLKDSFSNSSWEIEAQAFFILSDQHPNIISPHLTFSEGLDELLKLEGNFPEENNDKKSIYYLGVTSIKKRESGVLLTDKSRVSCPFPAPCLFTDIRVLQDSCSHFVENNINLGTSSKKTFASYMPQFQTCSTQTHKIMENKMCDLTV

SEQ ID NO: 187 - protein, 846 aa, synthetic construct
  REGION 1..846: note = hLEPR-MMH
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDEQKLISEEDLGGEQKLISEEDLHHHHHH

SEQ ID NO: 188 - protein, 844 aa, synthetic construct
  REGION 1..844: note = MfLEPR-MMH
FNLSYPITPWRFKLSCMPPNSTYDYFLLPAGLSKNTSNLNGHYETAVEFNSSDTHFSNLSKTTFHCCFRSEQDRNCSLCADNIEGKTFVSTVNSSVFQQMGANWNIQCWLKGDLKLFICYVESLFKNPFKNYKHKVHLLYVLPEVLEDSPLVPQKGSFQMVHCNCSVHERCECLVPVPTAKLNDTLLMCLKITSGGVIFQSPLMSVQPINMVKPDPPLGLRMEITDDGNLKISWSSPPLVPFPLQYEVKYSENSTTVIREADKIVSATSLLVDGILPGSSYEVQVRGKRLDGPGIWSDWSTPHVFTTQDVIYFPPKILTSVGSNVSFHCIYKNENKIVSSKKIVWWMNLAEKIPQSQYDVVSDHVSKVTFFNLNETKPRGKFTYDAVYCCNEHECHHRYAELYVIDVNINISCETDGHLTKMTCRWSTNTIQSLAGSTLQLRYRRSSLYCFDIPSIHPISKPKDCYLQSDGFYECVFQPIFLLSGYTMWIRINHPLGSLDSPPTCVLPDSVVKPLPPSSVKAEIIKNIGLLKISWEKPVFPENNLQFQIRYGLSGKEIQWKMYDVYDAKSKSVSLPVPDFCAVYAVQVRCKRSDGLGLWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVILSWILSPSDYKLMYFIIEWKNLNEDGEIKWLRISSSVKKYYIHDHFIPIEKYQFSLYPIFMEGVGKPKIINSFAQDNTEKHQNDEQKLISEEDLGGEQKLISEEDLHHHHHH

SEQ ID NO: 189 - protein, 1045 aa, synthetic construct
  REGION 1..1045: note = hLEPR-hFc
FNLSYPITPWRFKLSCMPPNSTYDYFLLPAGLSKNTSNSNGHYETAVEPKFNSSGTHFSNLSKTTFHCCFRSEQDRNCSLCADNIEGKTFVSTVNSLVFQQIDANWNIQCWLKGDLKLFICYVESLFKNLFRNYNYKVHLLYVLPEVLEDSPLVPQKGSFQMVHCNCSVHECCECLVPVPTAKLNDTLLMCLKITSGGVIFQSPLMSVQPINMVKPDPPLGLHMEITDDGNLKISWSSPPLVPFPLQYQVKYSENSTTVIREADKIVSATSLLVDSILPGSSYEVQVRGKRLDGPGIWSDWSTPRVFTTQDVIYFPPKILTSVGSNVSFHCIYKKENKIVPSKEIVWWMNLAEKIPQSQYDVVSDHVSKVTFFNLNETKPRGKFTYDAVYCCNEHECHHRYAELYVIDVNINISCETDGYLTKMTCRWSTSTIQSLAESTLQLRYHRSSLYCSDIPSIHPISEPKDCYLQSDGFYECIFQPIFLLSGYTMWIRINHSLGSLDSPPTCVLPDSVVKPLPPSSVKAEITINIGLLKISWEKPVFPENNLQFQIRYGLSGKEVQWKMYEVYDAKSKSVSLPVPDLCAVYAVQVRCKRLDGLGYWSNWSNPAYTVVMDIKVPMRGPEFWRIINGDTMKKEKNVTLLWKPLMKNDSLCSVQRYVINHHTSCNGTWSEDVGNHTKFTFLWTEQAHTVTVLAINSIGASVANFNLTFSWPMSKVNIVQSLSAYPLNSSCVIVSWILSPSDYKLMYFIIEWKNLNEDGEIKWLRISSSVKKYYIHDHFIPIEKYQFSLYPIFMEGVGKPKIINSFTQDDIEKHQSDDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 190 - protein, 830 aa, synthetic construct
  REGION 1..830: note = hGP130-mFc
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 191 - protein, 625 aa, synthetic construct
  REGION 1..625: note = hGP130-MMH
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEEQKLISEEDLGGEQKLISEEDLHHHHHH

SEQ ID NO: 192 - protein, 524 aa, synthetic construct
  REGION 1..524: note = hGP130 delta D1-MMH
LPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEEQKLISEEDLGGEQKLISEEDLHHHHHH

SEQ ID NO: 193 - protein, 319 aa, synthetic construct
  REGION 1..319: note = hGP130 delta D1-D3-MMH
APSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEEQKLISEEDLGGEQKLISEEDLHHHHHH

SEQ ID NO: 194 - protein, 625 aa, synthetic construct
  REGION 1..625: note = MfGP130-MMH
ELLDPCGYISPESPVVQLHSNFTAVCVLKEKCMDYFHVNANYIVWKTNHFTIPKEQYTIINRTASSVTFTDISSLNIQLTCNILTFGQLEQNVYGITIISGLPPEKPKNLSCIVNEGKKMRCEWNRGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIRLKYNIQYRTKDASTWSQIPPEDTASTRSSFTVQDLKPFTEYVFRICCMKEDGKGYWSDWSEEANGITYEDRPSKAPSFWYKIDPSHAQGYRTVQLMWKTLPPFEANGKILDYEVTLTRWKSHLQNYTVNDTKLTVNLTNDRYVATLTARNLVGKSDAAVLTIPACDFQATHPVMDLKAFPKDNMLWVEWTTPRESVKKYILEWCVLSDKAPCIADWQQEDGTVHRTHLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEEQKLISEEDLGGEQKLISEEDLHHHHHH

SEQ ID NO: 195 - protein, 624 aa, synthetic construct
  REGION 1..624: note = rGP130-MMH
QLVEPCGYIYPEFPVVQRGSNFTATCVLKEKCLQVYSVNATYIVWKTNHVAVPKEQVTVINRTASSVTFTDVVFQNVQLTCNILSFGQIEQNVYGITILSGYPPDIPTNLSCIVNEGKNMLCQWDPGRETYLETNYTLKSEWATEKFPDCRTKHGTSSCMMGYTPIYFVNIEVWVEAENALGNVSSEPINFDPVDKVKPSPPHNLSVTNSEELSSILKLAWVNSGLDSILRLKSDIQYRTKDASTWIQVPLEDTVSPRTSFTVQDLKPFTEYVFRIRSIKENGKGYWSDWSEEASGTTYEDRPSKAPSFWYKVNANHPQEYRSARLIWKTLPLSEANGKILDYEVVLTQSKSVSQTYTVNGTELIVNLTNNRYVASLAARNVVGKSPATVLTIPGSHFKASHPVVDLKAFPKDNLLWVEWTPPSKPVNKYILEWCVLSENSPCIPDWQQEDGTVNRTHLRGSLLESKCYLITVTPVFPGGPGSPESMKAYLKQAAPSKGPTVRTKKVGKNEAVLEWDHLPVDVQNGFIRNYSISYRTSVGKEMVVRVDSSHTEYTLSSLSSDTLYMVHMAAYTEEGGKDGPEFTFTTLKFAQGEIEEQKLISEEDLGGEQKLISEEDLHHHHHH

SEQ ID NO: 196 - protein, 623 aa, synthetic construct
  REGION 1..623: note = mGP130-MMH
QLLEPCGYIYPEFPVVQRGSNFTAICVLKEACLQHYYVNASYIVWKTNHAAVPREQVTVINRTTSSVTFTDVVLPSVQLTCNILSFGQIEQNVYGVTMLSGFPPDKPTNLTCIVNEGKNMLCQWDPGRETYLETNYTLKSEWATEKFPDCQSKHGTSCMVSYMPTYYVNIEVWVEAENALGKVSSESINFDPVDKVKPTPPYNLSVTNSEELSSILKLSWVSSGLGGLLDLKSDIQYRTKDASTWIQVPLEDTMSPRTSFTVQDLKPFTEYVFRIRSIKDSGKGYWSDWSEEASGTTYEDRPSRPPSFWYKTNPSHGQEYRSVRLIWKALPLSEANGKILDYEVILTQSKSVSQTYTVTGTELTVNLTNDRYVASLAARNKVGKSAAAVLTIPSPHVTAAYSVVNLKAFPKDNLLWVEWTPPPKPVSKYILEWCVLSENAPCVEDWQQEDATVNRTHLRGRLLESKCYQITVTPVFATGPGGSESLKAYLKQAAPARGPTVRTKKVGKNEAVLAWDQIPVDDQNGFIRNYSISYRTSVGKEMVVHVDSSHTEYTLSSLSSDTLYMVRMAAYTDEGGKDGPEFTFTTPKFAQGEIEEQKLISEEDLGGEQKLISEEDLHHHHHH

SEQ ID NO: 197 - protein, 824 aa, synthetic construct
  REGION 1..824: note = hGP130-hFc
ELLDPCGYISPESPVVQLHSNFTAVCVLKEKCMDYFHVNANYIVWKTNHFTIPKEQYTIINRTASSVTFTDIASLNIQLTCNILTFGQLEQNVYGITIISGLPPEKPKNLSCIVNEGKKMRCEWDGGRETHLETNFTLKSEWATHKFADCKAKRDTPTSCTVDYSTVYFVNIEVWVEAENALGKVTSDHINFDPVYKVKPNPPHNLSVINSEELSSILKLTWTNPSIKSVIILKYNIQYRTKDASTWSQIPPEDTASTRSSFTVQDLKPFTEYVFRIRCMKEDGKGYWSDWSEEASGITYEDRPSKAPSFWYKIDPSHTQGYRTVQLVWKTLPPFEANGKILDYEVTLTRWKSHLQNYTVNATKLTVNLTNDRYLATLTVRNLVGKSDAAVLTIPACDFQATHPVMDLKAFPKDNMLWVEWTTPRESVKKYILEWCVLSDKAPCITDWQQEDGTVHRTYLRGNLAESKCYLITVTPVYADGPGSPESIKAYLKQAPPSKGPTVRTKKVGKNEAVLEWDQLPVDVQNGFIRNYTIFYRTIIGNETAVNVDSSHTEYTLSSLTSDTLYMVRMAAYTDEGGKDGPEFTFTTPKFAQGEIEDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 198 - protein, 348 aa, synthetic construct
  REGION 1..348: note = hCNTFR-MMH
QRHSPQEAPHVQYERLGSDVTLPCGTANWDAAVTWRVNGTDLAPDLLNGSQLVLHGLELGHSGLYACFHRDSWHLRHQVLLHVGLPPREPVLSCRSNTYPKGFYCSWHLPTPTYIPNTFNVTVLHGSKIMVCEKDPALKNRCHIRYMHLFSTIKYKVSISVSNALGHNATAITFDEFTIVKPDPPENVVARPVPSNPRRLEVTWQTPSTWPDPESFPLKFFLRYRPLILDQWQHVELSDGTAHTITDAYAGKEYIIQVAAKDNEIGTWSDWSVAAHATPWTEEPRHLTTEAQAAETTTSTTSSLAPPPTTKICDPGELGSEQKLISEEDLGGEQKLISEEDLHHHHHH

(19) United States
(12) Patent Application Publication
(10) Pub. No.: US 2024/0158497 A1
(43) Pub. Date: May 16, 2024

(54) BISPECIFIC ANTIGEN BINDING MOLECULES THAT BIND LEPTIN RECEPTOR AND/OR GP130, AND METHODS OF USE THEREOF

(71) Applicant: Regeneron Pharmaceuticals, Inc., Tarrytown, NY (US)

(72) Inventors: Jesper GROMADA, Concord, MA (US); Panayiotis STEVIS, Glenmoore, PA (US); Judith ALTAREJOS, Armonk, NY (US)

(21) Appl. No.: 18/494,200

(22) Filed: Oct. 25, 2023

Related U.S. Application Data
(63) Continuation of application No. 16/222,800, filed on Dec. 17, 2018, now Pat. No. 11,834,500.
(60) Provisional application No. 62/635,406, filed on Feb. 26, 2018.
(60) Provisional application No. 62/607,137, filed on Dec. 18, 2017.

(51) Int. Cl. C07K 16/28; A61K 39/395; A61K 45/06; A61P 3/04; A61P 3/10; A61P 5/04; C07K 16/46

(52) U.S. Cl. CPC C07K 16/2803; A61K 39/3955; A61K 45/06; A61P 3/04; A61P 3/10; A61P 5/04; C07K 16/2866; C07K 16/2869; C07K 16/468; A61K 2039/505

(57) ABSTRACT

The present invention relates to antigen-binding molecules, including bispecific antigen-binding molecules that bind human GP130 and/or human leptin receptor (LEPR), and the use of such antigen-binding molecules for the treatment of conditions and disorders related to leptin deficiency or leptin resistance. The bispecific antigen-binding molecules of the present invention can be, e.g., bispecific antibodies comprising a first antigen-binding domain that specifically binds human GP130 and a second antigen-binding domain that specifically binds human LEPR. The bispecific antigen-binding molecules of the present invention are useful in therapeutic applications where induced leptin and/or LEPR-mediated signaling would be beneficial, e.g., in the treatment of obesity, lipodystrophies and other diseases and disorders associated with or caused by leptin deficiency or leptin resistance.

This application is a continuation of U.S. patent application Ser. No. 16/222,800, filed on Dec. 17, 2018, which claims the benefit of U.S. provisional patent application nos. 62/607,137, filed Dec. 18, 2017 and 62/635,406, filed Feb. 26, 2018; each of which is herein incorporated by reference in its entirety.

### FIELD OF THE INVENTION

The present invention relates to antigen-binding molecules, including bispecific antigen-binding molecules (e.g., bispecific antibodies) that bind human GP130 and/or human leptin receptor (LEPR), and the use of such antigen-binding molecules for the treatment of conditions and disorders related to leptin deficiency or leptin resistance.

### Sequence Listing

An official copy of the sequence listing is submitted concurrently with the specification via Patent Center. The contents of electronic sequence listing (10397US02 Sequence Listing ST26.xml; Size 270,336 bytes; and Date of Creation: Oct. 25, 2023) is part of the specification and is herein incorporated by reference in its entirety.

### BACKGROUND

Glycoprotein 130 (GP130) is a component of a receptor complex that also comprises CNTRF-alpha and LIFR-beta. Signaling through this receptor complex activates JAK/STAT signaling which, in certain biological contexts, results in reduced appetite, food intake and weight loss.

Leptin is a polypeptide hormone predominantly expressed by adipose tissue and is involved in the regulation of metabolism, energy balance and food intake. Leptin activity is mediated by interaction with, and signaling through, the leptin receptor. Leptin receptor, (also known as “LEPR,” “WSX,” “OB receptor,” “OB-R,” and “CD295”) is a single-pass transmembrane receptor of the class I cytokine receptor family with a large (818 amino acid) extracellular domain. Leptin deficiency, leptin resistance, and certain LEPR signaling-defective/signaling impaired mutations, are associated with obesity, type 2 diabetes, dyslipidemia, lipodystrophies, hepatic steatosis, non-alcoholic and alcoholic fatty liver diseases, severe insulin resistance, Leprechaunism/Donohue syndrome, Rabson-Mendenhall syndrome, and related complications. Therapeutic approaches to address leptin resistance, leptin deficiency, and hypoleptinemia (e.g., lipodystrophy) have mostly focused on the delivery of supplemental leptin or leptin analogues to affected individuals. Such approaches, however, have generally shown limited efficacy, particularly in leptin-resistant individuals, and are frequently associated with adverse side effects. Thus, a need exists in the art for alternative approaches to treating leptin resistance and other conditions associated with leptin deficiency or hypoleptinemia.

### BRIEF SUMMARY OF THE INVENTION

The present invention relates, in part, to the concept of antibody-mediated heterodimerization of the LEPR and GP130 to activate both receptors and thereby stimulate the anorexegenic effects associated with signaling through these receptors. Accordingly, the present invention provides antigen-binding molecules (e.g., antibodies and antigen-binding fragments of antibodies) that bind human GP130 and/or human leptin receptor (LEPR). According to certain embodiments, the present invention provides bispecific antigen-binding molecules comprising a first antigen-binding domain (D1) that specifically binds human GP130, and a second antigen-binding domain (D2) that specifically binds human leptin receptor (LEPR). The present invention includes LEPRxGP130 bispecific molecules (e.g., bispecific antibodies). In certain exemplary embodiments of the invention, the anti-GP130 antigen-binding domain (D1) and the anti-LEPR (D2) antigen-binding domain each comprise different, distinct heavy chain variable regions (HCVRs) paired with the same or a different light chain variable regions (LCVRs).

The antigen-binding molecules (e.g., bispecific antigen-binding molecules) of the present invention are useful, inter alia, for targeting cells that express LEPR and/or cells that express GP130, or both. According to certain embodiments, the bispecific antigen-binding molecules of the present invention are useful for physically linking LEPR and GP130 to one another on the surface of a cell in order to stimulate LEPR signaling. In this manner, the bispecific antigen binding molecules of the present invention may serve as LEPR agonists in a variety of therapeutic applications where leptin and/or LEPR-mediated signaling would be beneficial (e.g., in the treatment of obesity, lipodystrophies and other diseases and disorders associated with or caused by leptin deficiency or leptin resistance).

Other embodiments will become apparent from a review of the ensuing detailed description.

### BRIEF DESCRIPTION OF THE FIGURES

FIG. 1 shows the effects of LEPRxGP130 bispecific antibody treatment (open squares and open diamonds) on obese mice, fed a high fat diet, expressing human LEPR and human GP130, compared to treatment with isotype control antibody (closed circles). Antibodies were administered subcutaneously at 30 mg/kg on day 0 and day 7 (indicated by “Dose” and upward arrows), and the effects of antibody treatment on body weight (expressed in terms of average percent change in body weight from pre-dose) are plotted over time for each treatment group over time. Open squares represent mice treated with bsAb21236 (alternatively referred to as “H4H21236D”). Open diamonds represent mice treated with bsAb21237 (alternatively referred to as “H4H21237D”). (*) indicates P<0.05 isotype control vs. bsAb21236. (#) indicates P<0.05 isotype control vs. bsAb21237.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, the preferred methods and materials are now described. All patents, applications and non-patent publications mentioned in this specification are incorporated herein by reference in their entireties.

Gp130 Protein

The expressions “Glycoprotein 130,” “GP130,” “gp130,” and the like, as used herein, refer to the human GP130 protein comprising the amino acid sequence as set forth in SEQ ID NO:185 (see also UniProtKB Q17RA0). The expression “GP130” includes both monomeric and multimeric GP130 molecules. As used herein, the expression “monomeric human GP130” means a GP130 protein or portion thereof that does not contain or possess any multimerizing domains and that exists under normal conditions as a single GP130 molecule without a direct physical connection to another GP130 molecule. An exemplary monomeric GP130 molecule is the molecule referred to herein as “hGP130.mmh” comprising the amino acid sequence of SEQ ID NO:191 (see, e.g., Example 3, herein). As used herein, the expression “dimeric human GP130” means a construct comprising two GP130 molecules connected to one another through a linker, covalent bond, non-covalent bond, or through a multimerizing domain such as an antibody Fc domain. An exemplary dimeric GP130 molecule is the molecule referred to herein as “hGP130.hFc” comprising the amino acid sequence of SEQ ID NO:197 or “hGP130.mFc” comprising the amino acid sequence of SEQ ID NO:190 (see, e.g., Example 3, herein).

All references to proteins, polypeptides and protein fragments herein are intended to refer to the human version of the respective protein, polypeptide or protein fragment unless explicitly specified as being from a non-human species. Thus, the expression “GP130” means human GP130 unless specified as being from a non-human species, e.g., “mouse GP130,” “monkey GP130,” etc.

As used herein, the expression “cell surface-expressed GP130” means one or more GP130 protein(s), or the extracellular domain thereof, that is/are expressed on the surface of a cell in vitro or in vivo, such that at least a portion of a GP130 protein is exposed to the extracellular side of the cell membrane and is accessible to an antigen-binding portion of an antibody. A “cell surface-expressed GP130” can comprise or consist of a GP130 protein expressed on the surface of a cell which normally expresses GP130 protein. Alternatively, “cell surface-expressed GP130” can comprise or consist of GP130 protein expressed on the surface of a cell that normally does not express human GP130 on its surface but has been artificially engineered to express GP130 on its surface.

### Anti-Gp130 Antibodies and Antigen-Binding Fragments Thereof

According to one aspect of the present invention, anti-GP130 antibodies are provided (e.g., monospecific anti-GP130 antibodies). Exemplary anti-GP130 antibodies according to this aspect of the invention are listed in Tables 1 and 2 herein. Table 1 sets forth the amino acid sequence identifiers of the heavy chain variable regions (HCVRs), light chain variable regions (LCVRs), heavy chain complementarity determining regions (HCDR1, HCDR2 and HCDR3), and light chain complementarity determining regions (LCDR1, LCDR2 and LCDR3) of the exemplary anti-GP130 antibodies from which the bispecific antigen-binding molecules of the present invention may be derived. Table 2 sets forth the nucleic acid sequence identifiers of the HCVRs, LCVRs, HCDR1, HCDR2 HCDR3, LCDR1, LCDR2 and LCDR3 of the exemplary anti-GP130 antibodies.

The present invention provides antibodies or antigen-binding fragments thereof that specifically bind GP130, comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides antibodies or antigen-binding fragments thereof that specifically bind GP130, comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides antibodies or antigen-binding fragments thereof that specifically bind GP130, comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides antibodies, or antigen-binding fragments thereof, comprising an HCVR/LCVR amino acid sequence pair contained within any of the exemplary anti-GP130 antibodies listed in Table 1. In certain embodiments, the HCVR/LCVR amino acid sequence pair is SEQ ID NOs: 154/10.

The present invention also provides antibodies or antigen-binding fragments thereof that specifically bind GP130, comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind GP130, comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind GP130, comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind GP130, comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind GP130, comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind GP130, comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides antibodies or antigen-binding fragments thereof that specifically bind GP130, comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-GP130 antibodies listed in Table 1. In certain embodiments, the HCDR3/LCDR3 amino acid sequence pair is SEQ ID NOs: 160/16.

The present invention also provides antibodies or antigen-binding fragments thereof that specifically bind GP130, comprising a set of six CDRs (i.e., HCDR1-HCDR2-HCDR3-LCDR1-LCDR2-LCDR3) contained within any of the exemplary anti-GP130 antibodies listed in Table 1. In certain embodiments, the HCDR1-HCDR2-HCDR3-LCDR1-LCDR2-LCDR3 amino acid sequences set is selected from the group consisting of: SEQ ID NOs: 20-22-24-12-14-16, 28-30-32-12-14-16, 36-38-40-12-14-16, 44-46-48-12-14-16, 52-54-56-12-14-16, 60-62-64-12-14-16, 68-70-72-12-14-16, 76-78-80-12-14-16, 84-86-88-12-14-16, 92-94-96-12-14-16, 100-102-104-12-14-16, 108-110-112-12-14-16, 116-118-120-12-14-16, 124-126-128-12-14-16, 132-134-136-12-14-16, 140-142-144-12-14-16, 148-150-152-12-14-16 and 156-158-160-12-14-16.

In a related embodiment, the present invention provides antibodies, or antigen-binding fragments thereof that specifically bind GP130, comprising a set of six CDRs (i.e., HCDR1-HCDR2-HCDR3-LCDR1-LCDR2-LCDR3) contained within an HCVR/LCVR amino acid sequence pair as defined by any of the exemplary anti-GP130 antibodies listed in Table 1. For example, the present invention includes antibodies or antigen-binding fragments thereof that specifically bind GP130, comprising the HCDR1-HCDR2-HCDR3-LCDR1-LCDR2-LCDR3 amino acid sequences set contained within an HCVR/LCVR amino acid sequence pair of: SEQ ID NOs: 154/10.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

The present invention also provides nucleic acid molecules encoding anti-GP130 antibodies or portions thereof. For example, the present invention provides nucleic acid molecules encoding any of the HCVR amino acid sequences listed in Table 1; in certain embodiments the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto.

The present invention also provides nucleic acid molecules encoding any of the LCVR amino acid sequences listed in Table 1; in certain embodiments the nucleic acid molecule comprises a polynucleotide sequence selected from any of the LCVR nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR1 amino acid sequences listed in Table 1; in certain embodiments the nucleic acid molecule comprises a polynucleotide sequence selected from any of the HCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR2 amino acid sequences listed in Table 1; in certain embodiments the nucleic acid molecule comprises a polynucleotide sequence selected from any of the HCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the HCDR3 amino acid sequences listed in Table 1; in certain embodiments the nucleic acid molecule comprises a polynucleotide sequence selected from any of the HCDR3 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR1 amino acid sequences listed in Table 1; in certain embodiments the nucleic acid molecule comprises a polynucleotide sequence selected from any of the LCDR1 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR2 amino acid sequences listed in Table 1; in certain embodiments the nucleic acid molecule comprises a polynucleotide sequence selected from any of the LCDR2 nucleic acid sequences listed in Table 2, or a substantially similar sequence thereof having at least 90%, at least 95%, at least 98% or at least 99% sequence identity thereto.

The present invention also provides nucleic acid molecules encoding any of the LCDR3 amino acid sequences listed in Table 1; in certain embodiments the nucleic acid molecule comprises a polynucleotide sequence selected from any of the LCDR3 nucleic acid sequences listed in Table 2, or a substantially similar sequence thereof having at least 90%, at least 95%, at least 98% or at least 99% sequence identity thereto.

The present invention also provides nucleic acid molecules encoding an HCVR, wherein the HCVR comprises a set of three CDRs (i.e., HCDR1-HCDR2-HCDR3), wherein the HCDR1-HCDR2-HCDR3 amino acid sequence set is as defined by any of the exemplary anti-GP130 antibodies listed in Table 1.

The present invention also provides nucleic acid molecules encoding an LCVR, wherein the LCVR comprises a set of three CDRs (i.e., LCDR1-LCDR2-LCDR3), wherein the LCDR1-LCDR2-LCDR3 amino acid sequence set is as defined by any of the exemplary anti-GP130 antibodies listed in Table 1.

The present invention also provides nucleic acid molecules encoding both an HCVR and an LCVR, wherein the HCVR comprises an amino acid sequence of any of the HCVR amino acid sequences listed in Table 1, and wherein the LCVR comprises an amino acid sequence of any of the LCVR amino acid sequences listed in Table 1. In certain embodiments, the nucleic acid molecule comprises a polynucleotide sequence selected from any of the HCVR nucleic acid sequences listed in Table 2, or a substantially similar sequence thereof having at least 90%, at least 95%, at least 98% or at least 99% sequence identity thereto, and a polynucleotide sequence selected from any of the LCVR nucleic acid sequences listed in Table 2, or a substantially similar sequence thereof having at least 90%, at least 95%, at least 98% or at least 99% sequence identity thereto. In certain embodiments according to this aspect of the invention, the nucleic acid molecule encodes an HCVR and LCVR, wherein the HCVR and LCVR are both derived from the same anti-GP130 antibody listed in Table 1.

The present invention also provides recombinant expression vectors capable of expressing a polypeptide comprising a heavy or light chain variable region of an anti-GP130 antibody. For example, the present invention includes recombinant expression vectors comprising any of the nucleic acid molecules mentioned above, i.e., nucleic acid molecules encoding any of the HCVR, LCVR, and/or CDR sequences as set forth in Table 1. Also included within the scope of the present invention are host cells into which such vectors have been introduced, as well as methods of producing the antibodies or portions thereof by culturing the host cells under conditions permitting production of the antibodies or antibody fragments, and recovering the antibodies and antibody fragments so produced.

The present invention includes anti-GP130 antibodies having a modified glycosylation pattern. In some embodiments, modification to remove undesirable glycosylation sites may be useful, or an antibody lacking a fucose moiety present on the oligosaccharide chain, for example, to increase antibody dependent cellular cytotoxicity (ADCC) function (see Shield et al. (2002) JBC 277:26733). In other applications, modification of galactosylation can be made in order to modify complement dependent cytotoxicity (CDC).

LEPR Protein

The expression “leptin receptor,” “LEPR,” and the like, as used herein, refers to the human leptin receptor, comprising the amino acid sequence as set forth in SEQ ID NO:186 (see also UniProtKB/Swiss-Prot Accession No. P48357). Alternative names for LEPR used in the scientific literature include “OB receptor,” “OB-R,” and “CD295.” LEPR is also referred to as “WSX” (see, e.g., U.S. Pat. No. 7,524,937). The expression “LEPR” includes both monomeric and multimeric LEPR molecules. As used herein, the expression “monomeric human LEPR” means a LEPR protein or portion thereof that does not contain or possess any multimerizing domains and that exists under normal conditions as a single LEPR molecule without a direct physical connection to another LEPR molecule. An exemplary monomeric LEPR molecule is the molecule referred to herein as “hLEPR.mmh” comprising the amino acid sequence of SEQ ID NO:187 (see, e.g., Example 10, herein). As used herein, the expression “dimeric human LEPR” means a construct comprising two LEPR molecules connected to one another through a linker, covalent bond, non-covalent bond, or through a multimerizing domain such as an antibody Fc domain. An exemplary dimeric LEPR molecule is the molecule referred to herein as “hLEPR.hFc” comprising the amino acid sequence of SEQ ID NO:189 (see, e.g., Example 10, herein).

All references to proteins, polypeptides and protein fragments herein are intended to refer to the human version of the respective protein, polypeptide or protein fragment unless explicitly specified as being from a non-human species. Thus, the expression “LEPR” means human LEPR unless specified as being from a non-human species, e.g., “mouse LEPR,” “monkey LEPR,” etc.

As used herein, the expression “cell surface-expressed LEPR” means one or more LEPR protein(s), or the extracellular domain thereof, that is/are expressed on the surface of a cell in vitro or in vivo, such that at least a portion of a LEPR protein is exposed to the extracellular side of the cell membrane and is accessible to an antigen-binding portion of an antibody. A “cell surface-expressed LEPR” can comprise or consist of a LEPR protein expressed on the surface of a cell which normally expresses LEPR protein. Alternatively, “cell surface-expressed LEPR” can comprise or consist of LEPR protein expressed on the surface of a cell that normally does not express human LEPR on its surface but has been artificially engineered to express LEPR on its surface.

Several isoforms of the LEPR are generated through alternative splicing, resulting in a long isoform b (LEPR-b) and several short forms, including isoform a (LEPR-a) which shows the highest and broadest expression pattern (Tartaglia LA. The leptin receptor. J Biol Chem 1997; 272: 6093-6096). LEPR-b is the predominant isoform expressed in the brain, while LEPR-a is broadly expressed in the liver. All the isoforms share the same extracellular domain, transmembrane region and a short stretch of the cytoplasmic domain, containing the Box 1 region, followed by a variable region. The long form contains intracellular sequence motifs required for mediating all the signaling capabilities of leptin whereas the short forms are lacking these regions. The extracellular domain of the short forms is identical to the signaling competent long form.

Lepr x Gp130 Bispecific Antigen-Binding Molecules

The present invention is based on the concept of stimulating LEPR and GP130 signaling by bridging LEPR and GP130 on the surface of a cell. In particular, the present invention relates to the premise that a bispecific antigen-binding molecule, such as a LEPRxGP130 bispecific antibody (as described in detail elsewhere herein), is capable of stimulating LEPR-dependent signaling of STAT3 by bringing GP130 into relative proximity of the leptin receptor on the surface of cells, even in the absence leptin. In this manner, the bispecific antigen-binding molecules of the present invention may serve as LEPR agonists which may find use in therapeutic contexts where induced leptin/LEPR signaling is beneficial and/or desirable.

Accordingly, the present invention provides bispecific antigen binding molecules comprising a first antigen-binding domain (also referred to herein as “D1”) that binds human GP130, and a second antigen-binding domain (also referred to herein as “D2”) that binds human LEPR. According to the present invention, and as demonstrated in the working examples herein, the simultaneous binding LEPR and GP130 by the bispecific antigen-binding molecules of the invention results in stimulation of LEPR signaling.

The bispecific antigen-binding molecules of the present invention, may be referred to herein as “LEPRxGP130 bispecific antibodies,” or other related terminology.

LEPRxGP130 bispecific antigen-binding molecules of the present invention may be constructed using the antigen-binding domains derived from monospecific (conventional) anti-LEPR antibodies and anti-GP130 antibodies. For example, a collection of monoclonal, monospecific, anti-LEPR and/or anti-GP130 antibodies may be produced using standard methods known in the art, and the antigen-binding domains thereof can be used to construct LEPRxGP130 bispecific antigen-binding molecules (e.g., bispecific antibodies) using conventional techniques known in the art.

Exemplary anti-LEPR antibodies that can be used in the context of the present invention to produce LEPRxGP130 bispecific antigen binding molecules include any of the anti-LEPR antibodies described in US Patent Application Publication No. 2017/0101477, the disclosure of which is incorporated herein in its entirety. Anti-LEPR antibodies that can be used to construct the LEPRxGP130 bispecific antigen-binding molecules of the present invention may be agonist antibodies, i.e., antibodies that bind human LEPR and activate LEPR signaling. In other embodiments, anti-LEPR antibodies that can be used to construct LEPRxGP130 bispecific antigen-binding molecules may be potentiating antibodies, i.e., antibodies that enhance leptin-mediated signaling through LEPR. Anti-LEPR antibodies that are useful for constructing LEPRxGP130 bispecific antigen-binding molecules may be antibodies that are able to bind LEPR in complexed with leptin. Such antibodies include those that bind LEPR and do not block the LEPR:leptin interaction. Alternatively, anti-LEPR antibodies that are useful for constructing LEPRxGP130 bispecific antigen-binding molecules may be antibodies that compete with leptin for binding to LEPR, and/or only bind LEPR in the absence of leptin. Non-limiting examples of particular anti-LEPR antibodies that can be used to construct LEPRxGP130 bispecific antigen-binding molecules include the anti-LEPR antibodies referred to herein as “mAb18445” and “mAb18446”.

In some embodiments, the LEPRxGP130 bispecific antigen-binding molecule is derived from an anti-LEPR antibody that potentiates leptin-mediated signaling in vitro through the LEPR-b isoform.

In some embodiments, the LEPRxGP130 bispecific antigen-binding molecule is derived from an anti-LEPR antibody that does not activate leptin-mediated signaling in vitro through the LEPR-a isoform.

Exemplary anti-GP130 antibodies that can be used in the context of the present invention to produce LEPRxGP130 bispecific antigen binding molecules include any of the anti-GP130 antibodies described elsewhere herein. Anti-GP130 antibodies that are useful for constructing LEPRxGP130 bispecific antigen-binding molecules include anti-GP130 antibodies with one or more of the following properties: binds monkey GP130, does not bind mouse or rat GP130, binds to an epitope within the FNIII domain of GP130, does not inhibit GP130 ligand-mediated signaling, and/or does not activate GP130 signaling in the absence of a GP130 ligand. GP130 ligands include, e.g., human oncostatin M (OSM), human leukemia inhibitory factor (LIF), and human ciliary neurotrophic factor (CNTF). A non-limiting example of a particular anti-GP130 antibody that can be used to construct LEPRxGP130 bispecific antigen-binding molecules include the anti-GP130 antibody referred to herein as “mAb16683”.

According to the present invention, a bispecific antigen-binding molecule can be a single multifunctional polypeptide, or it can be a multimeric complex of two or more polypeptides that are covalently or non-covalently associated with one another. As will be made evident by the present disclosure, any antigen binding construct which has an antigen-binding domain that specifically binds human LEPR and an antigen-binding domain that specifically binds human GP130 is regarded as a “bispecific antigen-binding molecule.” Any of the bispecific antigen-binding molecules of the invention, or variants thereof, may be constructed using standard molecular biological techniques (e.g., recombinant DNA and protein expression technology), as will be known to a person of ordinary skill in the art.

The bispecific antigen-binding molecules of the invention may be “isolated.” An “isolated bispecific antigen-binding molecule,” as used herein, means a bispecific antigen-binding molecule that has been identified and separated and/or recovered from at least one component of its natural environment. For example, a bispecific antibody that has been separated or removed from at least one component of an organism, or from a tissue or cell in which the antibody is produced, is an “isolated bispecific antibody” for purposes of the present invention. An isolated bispecific antigen-binding molecule also includes molecules in situ within a recombinant cell. Isolated bispecific antigen-binding molecules are molecules that have been subjected to at least one purification or isolation step. According to certain embodiments, an isolated bispecific antigen-binding molecule may be substantially free of other cellular material and/or chemicals.

### Antigen-Binding Domains

The bispecific antigen-binding molecules of the present invention comprise two separate antigen-binding domains (D1 and D2). As used herein, the expression “antigen-binding domain” means any peptide, polypeptide, nucleic acid molecule, scaffold-type molecule, peptide display molecule, or polypeptide-containing construct that is capable of specifically binding a particular antigen of interest (e.g., human LEPR or human GP130). The term “specifically binds” or the like, as used herein in reference to an antigen-binding domain, means that the antigen-binding domain is capable of forming a complex with a particular antigen and does not bind other unrelated antigens under ordinary test conditions. “Unrelated antigens” are proteins, peptides or polypeptides that have less than 95% amino acid identity to one another.

Exemplary categories of antigen-binding domains that can be used in the context of the present invention include antibodies, antigen-binding portions of antibodies, peptides that specifically interact with a particular antigen (e.g., peptibodies), receptor molecules that specifically interact with a particular antigen, proteins comprising a ligand-binding portion of a receptor that specifically binds a particular antigen, antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, 2011, Curr. Opin. Biotechnol. 22:849-857, and references cited therein]), and aptamers or portions thereof.

Methods for determining whether two molecules specifically bind one another are well known in the art and include, for example, equilibrium dialysis, surface plasmon resonance, and the like. The term “surface plasmon resonance”, as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

As indicated above, an “antigen-binding domain” (D1 and/or D2) may comprise or consist of an antibody or antigen-binding fragment of an antibody. The term “antibody,” as used herein, means any antigen-binding molecule or molecular complex comprising at least one complementarity determining region (CDR) that specifically binds to or interacts with a particular antigen (e.g., human MET). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the antibodies of the invention (or antigen-binding portion thereof) may be identical to the human germline sequences, or may be naturally or artificially modified. An amino acid consensus sequence may be defined based on a side-by-side analysis of two or more CDRs.

The D1 and/or D2 components of the bispecific antigen-binding molecules of the present invention may comprise or consist of antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}—V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The bispecific antigen-binding molecules of the present invention may comprise or consist of human antibodies and/or recombinant human antibodies, or fragments thereof. The term “human antibody”, as used herein, includes antibodies having variable and constant regions derived from human germline immunoglobulin sequences. Human antibodies may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

The bispecific antigen-binding molecules of the present invention may comprise or consist of recombinant human antibodies or antigen-binding fragments thereof. The term “recombinant human antibody”, as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

Methods for making bispecific antibodies are known in the art and may be used to construct bispecific antigen-binding molecules of the present invention. Exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab e bispecific formats (see, e.g., Klein et al. 2012, mAbs 4:6, 1-11, and references cited therein, for a review of the foregoing formats).

Exemplary antigen-binding domains (D1 and D2) that can be included in the LEPRxGP130 bispecific antigen-binding molecules of the present invention include antigen-binding domains derived from any of the anti-LEPR and/or anti-GP130 antibodies disclosed herein or otherwise known in the art.

For example, the present invention includes LEPRxGP130 bispecific antigen-binding molecules comprising a D1 (GP130-binding) antigen-binding domain comprising an HCVR comprising an amino acid sequence selected from any of the HCVR amino acid sequences listed in Table 1, or a substantially similar sequence thereof having at least 90%, at least 95%, at least 98% or at least 99% sequence identity thereto.

The present invention also provides LEPRxGP130 bispecific antigen-binding molecules comprising a D1 (GP130-binding) antigen-binding domain comprising an LCVR comprising an amino acid sequence selected from any of the LCVR amino acid sequences listed in Table 1, or a substantially similar sequence thereof having at least 90%, at least 95%, at least 98% or at least 99% sequence identity thereto.

The present invention also provides LEPRxGP130 bispecific antigen-binding molecules comprising a D1 (GP130-binding) antigen-binding domain comprising an HCVR and an LCVR amino acid sequence pair (HCVR/LCVR) comprising any of the HCVR amino acid sequences listed in Table 1 paired with any of the LCVR amino acid sequences listed in Table 1. According to certain embodiments, the present invention provides LEPRxGP130 bispecific antigen-binding molecules comprising a D1 (GP130-binding) domain comprising an HCVR/LCVR amino acid sequence pair contained within any of the exemplary anti-MET antibodies listed in Table 1.

The present invention also provides LEPRxGP130 bispecific antigen-binding molecules comprising a D1 (GP130-binding) antigen-binding domain comprising a heavy chain CDR1 (HCDR1) comprising an amino acid sequence selected from any of the HCDR1 amino acid sequences listed in Table 1 or a substantially similar sequence thereof having at least 90%, at least 95%, at least 98% or at least 99% sequence identity.

The present invention also provides LEPRxGP130 bispecific antigen-binding molecules comprising a D1 (GP130-binding) antigen-binding domain comprising a heavy chain CDR2 (HCDR2) comprising an amino acid sequence selected from any of the HCDR2 amino acid sequences listed in Table 1 or a substantially similar sequence thereof having at least 90%, at least 95%, at least 98% or at least 99% sequence identity.

The present invention also provides LEPRxGP130 bispecific antigen-binding molecules comprising a D1 (GP130-binding) antigen-binding domain comprising a heavy chain CDR3 (HCDR3) comprising an amino acid sequence selected from any of the HCDR3 amino acid sequences listed in Table 1 or a substantially similar sequence thereof having at least 90%, at least 95%, at least 98% or at least 99% sequence identity.

The present invention also provides LEPRxGP130 bispecific antigen-binding molecules comprising a D1 (GP130-binding) antigen-binding domain comprising a light chain CDR1 (LCDR1) comprising an amino acid sequence selected from any of the LCDR1 amino acid sequences listed in Table 1 or a substantially similar sequence thereof having at least 90%, at least 95%, at least 98% or at least 99% sequence identity.

The present invention also provides LEPRxGP130 bispecific antigen-binding molecules comprising a D1 (GP130-binding) antigen-binding domain comprising a light chain CDR2 (LCDR2) comprising an amino acid sequence selected from any of the LCDR2 amino acid sequences listed in Table 1 or a substantially similar sequence thereof having at least 90%, at least 95%, at least 98% or at least 99% sequence identity.

The present invention also provides LEPRxGP130 bispecific antigen-binding molecules comprising a D1 (GP130-binding) antigen-binding domain comprising a light chain CDR3 (LCDR3) comprising an amino acid sequence selected from any of the LCDR3 amino acid sequences listed in Table 1 or a substantially similar sequence thereof having at least 90%, at least 95%, at least 98% or at least 99% sequence identity.

The present invention also provides LEPRxGP130 bispecific antigen-binding molecules comprising a D1 (GP130-binding) antigen-binding domain comprising an HCDR3 and an LCDR3 amino acid sequence pair (HCDR3/LCDR3) comprising any of the HCDR3 amino acid sequences listed in Table 1 paired with any of the LCDR3 amino acid sequences listed in Table 1. According to certain embodiments, the present invention provides antibodies, or antigen-binding fragments thereof, comprising an HCDR3/LCDR3 amino acid sequence pair contained within any of the exemplary anti-MET antibodies listed in Table 1.

The present invention also provides LEPRxGP130 bispecific antigen-binding molecules comprising a D1 (GP130-binding) antigen-binding domain comprising a set of six CDRs (i.e., HCDR1-HCDR2-HCDR3-LCDR1-LCDR2-LCDR3) contained within any of the exemplary anti-MET antibodies listed in Table 1.

In a related embodiment, the present invention provides LEPRxGP130 bispecific antigen-binding molecules comprising a D1 (GP130-binding) antigen-binding domain comprising a set of six CDRs (i.e., HCDR1-HCDR2-HCDR3-LCDR1-LCDR2-LCDR3) contained within an HCVR/LCVR amino acid sequence pair as defined by any of the exemplary anti-MET antibodies listed in Table 1.

The present invention includes LEPRxGP130 bispecific antigen-binding molecules comprising a D2 (LEPR-binding) antigen-binding domain comprising a variable domain (HCVR and/or LCVR), and/or complementarity determining region (HCDR1, HCDR2, HCDR3, LCDR1, LCDR2, and/or LCDR3), derived from any of the anti-LEPR antibodies described herein, described in US Patent Application Publication No. 2017/0101477, the disclosure of which is incorporated herein in its entirety, or otherwise known in the art.

As non-limiting illustrative examples, the present invention includes LEPRxGP130 bispecific antigen binding molecules comprising a D1 (GP130-binding) antigen-binding domain and a D2 (LEPR-binding) antigen-binding domain, wherein the D1 antigen binding domain comprises an HCVR/LCVR amino acid sequence pair of SEQ ID NOs: 154/10, or a set of heavy and light chain CDRs (HCDR1-HCDR2-HCDR3-LCDR1-LCDR2-LCDR3) comprising SEQ ID NOs: 156-158-160-12-14-16, and wherein the D2 (LEPR-binding) antigen-binding domain comprises an HCVR/LCVR amino acid sequence pair of SEQ ID NOs: 170/10 or 178/10, or a set of heavy and light chain CDRs (HCDR1-HCDR2-HCDR3-LCDR1-LCDR2-LCDR3) comprising SEQ ID NOs: 172-174-176-12-14-16, or 180-182-184-12-14-16. An exemplary LEPRxGP130 bispecific antibody having these sequence characteristics is the bispecific antibody designated bsAb21236, which comprises a D1 derived from mAb16683 and a D2 derived from mAb18445. Another exemplary LEPRxGP130 bispecific antibody having these sequence characteristics is the bispecific antibody designated bsAb21237, which comprises a D1 derived from mAb16683 and a D2 derived from mAb18446. Other specific examples of bispecific antibodies of the present invention are set forth in Example 9, Table 20 herein.

### Multimerizing Components

The bispecific antigen-binding molecules of the present invention, in certain embodiments, may also comprise one or more multimerizing component(s). The multimerizing components can function to maintain the association between the antigen-binding domains (D1 and D2). As used herein, a “multimerizing component” is any macromolecule, protein, polypeptide, peptide, or amino acid that has the ability to associate with a second multimerizing component of the same or similar structure or constitution. For example, a multimerizing component may be a polypeptide comprising an immunoglobulin C_{H}3 domain. A non-limiting example of a multimerizing component is an Fc portion of an immunoglobulin, e.g., an Fc domain of an IgG selected from the isotypes IgG1, IgG2, IgG3, and IgG4, as well as any allotype within each isotype group. In certain embodiments, the multimerizing component is an Fc fragment or an amino acid sequence of 1 to about 200 amino acids in length containing at least one cysteine residues. In other embodiments, the multimerizing component is a cysteine residue, or a short cysteine-containing peptide. Other multimerizing domains include peptides or polypeptides comprising or consisting of a leucine zipper, a helix-loop motif, or a coiled-coil motif.

In certain embodiments, the bispecific antigen-binding molecules of the present invention comprise two multimerizing domains, M1 and M2, wherein D1 is attached to M1 and D2 is attached to M2, and wherein the association of M1 with M2 facilitates the physical linkage of D1 and D2 to one another in a single bispecific antigen-binding molecule. In certain embodiments, M1 and M2 are identical to one another. For example, M1 can be an Fc domain having a particular amino acid sequence, and M2 is an Fc domain with the same amino acid sequence as M1. Alternatively, M1 and M2 may differ from one another at one or more amino acid position. For example, M1 may comprise a first immunoglobulin (Ig) C_{H}3 domain and M2 may comprise a second Ig C_{H}3 domain, wherein the first and second Ig C_{H}3 domains differ from one another by at least one amino acid, and wherein at least one amino acid difference reduces binding of the targeting construct to Protein A as compared to a reference construct having identical M1 and M2 sequences. In one embodiment, the Ig C_{H}3 domain of M1 binds Protein A and the Ig C_{H}3 domain of M2 contains a mutation that reduces or abolishes Protein A binding such as an H95R modification (by IMGT exon numbering; H435R by EU numbering). The C_{H}3 of M2 may further comprise a Y96F modification (by IMGT; Y436F by EU). Further modifications that may be found within the C_{H}3 of M2 include: D16E, L18M, N44S, K52N, V57M, and V821 (by IMGT; D356E, L358M, N384S, K392N, V397M, and V4221 by EU) in the case of an IgG1 Fc domain; N44S, K52N, and V821 (IMGT; N384S, K392N, and V4221 by EU) in the case of an IgG2 Fc domain; and Q15R, N44S, K52N, V57M, R69K, E79Q, and V821 (by IMGT; Q355R, N384S, K392N, V397M, R409K, E419Q, and V4221 by EU) in the case of an IgG4 Fc domain.

### Variants

The bispecific antigen-binding molecules disclosed herein, or the antigen-binding domains thereof (D1 and/or D2) may comprise one or more amino acid substitutions, insertions and/or deletions in the framework and/or CDR regions of the heavy and light chain variable domains as compared to the corresponding germline sequences from which the antigen-binding proteins or antigen-binding domains were derived. Such mutations can be readily ascertained by comparing the amino acid sequences disclosed herein to germline sequences available from, for example, public antibody sequence databases. The present invention includes bispecific antigen-binding molecules disclosed herein, or the antigen-binding domains thereof (D1 and/or D2), which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous bispecific antigen-binding molecules, or antigen-binding domains thereof (D1 and/or D2), which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the bispecific antigen-binding molecules, or the antigen-binding domains thereof (D1 and/or D2), of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, bispecific antigen-binding molecules, or the antigen-binding domains thereof (D1 and/or D2), that contain one or more germline mutations can be easily tested for one or more desired property such as, improved binding specificity, increased binding affinity, improved or enhanced antagonistic or agonistic biological properties (as the case may be), reduced immunogenicity, etc. bispecific antigen-binding molecules, or the antigen-binding domains thereof (D1 and/or D2), obtained in this general manner are encompassed within the present invention.

The present invention also includes anti-LEPR antibodies, anti-GP130 antibodies, and bispecific antigen-binding molecules comprising variants of any of the HCVR, LCVR, and/or CDR amino acid sequences disclosed herein. Exemplary variants included within this aspect of the invention include variants of any of the HCVR, LCVR, and/or CDR amino acid sequences disclosed herein having one or more conservative substitutions. For example, the present invention includes anti-LEPR antibodies, anti-GP130 antibodies, and LEPRxGP130 bispecific antigen-binding molecules having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences set herein.

Exemplary variants included within this aspect of the invention also include variants having substantial sequence identity to any of the HCVR, LCVR, and/or CDR amino acid sequences disclosed herein. As used herein in the context of amino acid sequences, the term “substantial identity” or “substantially identical” means that two amino acid sequences, when optimally aligned, such as by the programs GAP or BESTFIT using default gap weights, share at least 95%, 98% or 99% sequence identity. In certain embodiments, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity may be adjusted upwards to correct for the conservative nature of the substitution. Means for making this adjustment are well-known to those of skill in the art. See, e.g., Pearson (1994) Methods Mol. Biol. 24: 307-331, herein incorporated by reference. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443-1445, herein incorporated by reference. A “moderately conservative” replacement is any change having a nonnegative value in the PAM250 log-likelihood matrix.

Sequence identity between two different amino acid sequences is typically measured using sequence analysis software. Sequence analysis software matches similar sequences using measures of similarity assigned to various substitutions, deletions and other modifications, including conservative amino acid substitutions. For instance, GCG software contains programs such as Gap and Bestfit which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA using default or recommended parameters, a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402, each herein incorporated by reference.

LEPRxGP130 Bispecific Antigen-Binding Molecules Comprising Fc Variants

According to certain embodiments of the present invention, LEPRxGP130 bispecific antigen binding proteins are provided comprising an Fc domain comprising one or more mutations which enhance or diminish antibody binding to the FcRn receptor, e.g., at acidic pH as compared to neutral pH. For example, the present invention includes LEPRxGP130 bispecific antigen binding proteins comprising a mutation in the C_{H}2 or a C_{H}3 region of the Fc domain, wherein the mutation(s) increases the affinity of the Fc domain to FcRn in an acidic environment (e.g., in an endosome where pH ranges from about 5.5 to about 6.0). Such mutations may result in an increase in serum half-life of the antibody when administered to an animal. Non-limiting examples of such Fc modifications include, e.g., a modification at position 250 (e.g., E or Q); 250 and 428 (e.g., L or F); 252 (e.g., L/Y/F/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

For example, the present invention includes LEPRxGP130 bispecific antigen binding proteins comprising an Fc domain comprising one or more pairs or groups of mutations selected from the group consisting of: 250Q and 248L (e.g., T250Q and M248L); 252Y, 254T and 256E (e.g., M252Y, S254T and T256E); 428L and 434S (e.g., M428L and N434S); and 433K and 434F (e.g., H433K and N434F). All possible combinations of the foregoing Fc domain mutations, and other mutations within the antibody variable domains disclosed herein, are contemplated within the scope of the present invention.

Biological Characteristics of the LEPRxGP130 Antigen-Binding Molecules of the Invention

The present invention includes LEPRxGP130 bispecific antigen-binding molecules that bind human LEPR with high affinity. For example, the present invention includes LEPRxGP130 antigen-binding molecules that bind monomeric human LEPR (e.g., hLEPR.mmh) with a K D of less than about 110 nM as measured by surface plasmon resonance at 25° C., e.g., using an assay format as defined in Example 10 herein, or a substantially similar assay. According to certain embodiments, LEPRxGP130 bispecific antigen-binding molecules are provided that bind monomeric human LEPR (e.g., hLEPR.mmh) with a dissociative half-life (t ½) of greater than about 3 minutes as measured by surface plasmon resonance at 25° C., e.g., using an assay format as defined in1 Example 10 herein, or a substantially similar assay.

The present invention includes LEPRxGP130 bispecific antigen-binding molecules that bind human GP130 with high affinity. For example, the present invention includes LEPRxGP130 antigen-binding molecules that bind monomeric human GP130 (e.g., hGP130.mmh) with a K_{D} of less than about 150 nM as measured by surface plasmon resonance at 25° C., e.g., using an assay format as defined in Example 10 herein, or a substantially similar assay. According to certain embodiments, LEPRxGP130 bispecific antigen-binding molecules are provided that bind monomeric human GP130 (e.g., hGP130.mmh) with a dissociative half-life (t ½) of greater than about 2.5 minutes as measured by surface plasmon resonance at 25° C., e.g., using an assay format as defined in Example 10 herein, or a substantially similar assay.

The present invention includes LEPRxGP130 bispecific antigen-binding molecules that are capable of binding cells that express human LEPR. In some aspects, the LEPRxGP130 bispecific antigen-binding molecules are capable of binding cells that express human LEPR, isoform b. In certain embodiments, LEPRxGP130 bispecific antigen-binding proteins are provided that bind cells expressing human LEPR in the presence and/or absence of leptin. The present invention includes LEPRxGP130 bispecific antigen-binding molecules that are capable of binding cells that express human GP130. Cell binding by a bispecific antigen-binding molecule of the present invention may be assessed by fluorescence activated cell sorting (FACS) on cells expressing LEPR and or GP130, e.g., using an assay format as defined in Example 11 herein, or a substantially similar assay.

The present invention includes LEPRxGP130 bispecific antigen-binding molecules that activate GP130-mediated cell signaling. In certain embodiments, the present invention includes LEPRxGP130 bispecific antigen-binding molecules that activate GP130-mediated cell signaling with a potency that is at least 20% the degree of activation observed by treatment with a GP130 ligand. For example, the present invention includes LEPRxGP130 bispecific antigen-binding molecules that activate GP130-mediated cell signaling with a potency that is at least 20% or 25% the degree of activation observed by treatment with human oncostatin M (OSM) under the same or similar experimental assay condition. Activation of GP130-mediated cell signaling by a bispecific antigen-binding molecule of the present invention may be assessed by an in vitro cell signaling assay, e.g., using an assay format as defined in Example 12 herein, or a substantially similar assay.

The present invention includes LEPRxGP130 bispecific antigen-binding molecules that specifically, or preferentially, activate signaling through LEPR isoform ‘b’ (long form) and do not substantially activate signaling through LEPR isoform ‘a’ (short form). According to certain embodiments, LEPRxGP130 bispecific antigen-binding molecules are provided that specifically, or preferentially, potentiate leptin signaling through LEPR isoform ‘b’ (long form) and do not substantially potentiate leptin signaling through LEPR isoform ‘a’ (short form). Activation or potentiating of signaling through LEPR isoform ‘b’ and/or LEPR isoform ‘a’ may be assessed by an in vitro assay using a reporter cell line that specifically expresses LEPR isoform ‘b’ or LEPR isoform ‘a’, e.g., using an assay format as defined in Example 14 herein, or a substantially similar assay.

The present invention includes LEPRxGP130 bispecific antigen-binding molecules that cause a reduction in body weight when administered to an animal. For example, the present invention includes LEPRxGP130 bispecific antigen-binding molecules that cause a 1% to 4% reduction in body weight in animals 2 to 14 days following administration of the bispecific antigen-binding molecule in a therapeutically effective dose to the animal. Weight loss induction by the bispecific antigen-binding molecules of the invention may be assessed using a genetically engineered model system, e.g., using an in vivo model as set forth in Example 13 herein, or a substantially similar model.

The bispecific antigen-binding molecules of the present invention may possess one or more of the aforementioned biological characteristics, or any combination thereof. The foregoing list of biological characteristics of the bispecific antigen-binding molecules of the invention is not intended to be exhaustive. Other biological characteristics of the bispecific antigen-binding molecules of the present invention will be evident to a person of ordinary skill in the art from a review of the present disclosure including the working Examples herein.

Epitope Mapping, Binding Domains, and Related Technologies

The epitope to which the antibodies and antigen-binding domains of the present invention bind may consist of a single contiguous sequence of 3 or more (e.g., 3, 4, 5, 6, 7, 8, 9, 10, 11, 12, 13, 14, 15, 16, 17, 18, 19, 20 or more) amino acids of a LEPR or GP130 protein. Alternatively, the relevant epitope may consist of a plurality of non-contiguous amino acids (or amino acid sequences) of the target protein.

Various techniques known to persons of ordinary skill in the art can be used to determine the epitope on LEPR and/or GP130 with which the antibodies and antigen-binding domains of the present invention interact. Exemplary techniques that can be used to determine an epitope or binding domain of a particular antibody or antigen-binding domain include, e.g., point mutagenesis (e.g., alanine scanning mutagenesis, arginine scanning mutagenesis, etc.), peptide blots analysis (Reineke, 2004, Methods Mol Biol 248:443-463), protease protection, and peptide cleavage analysis. In addition, methods such as epitope excision, epitope extraction and chemical modification of antigens can be employed (Tomer, 2000, Protein Science 9:487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water to allow hydrogen-deuterium exchange to occur at all residues except for the residues protected by the antibody (which remain deuterium-labeled). After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267(2):252-259; Engen and Smith (2001) Anal. Chem. 73:256A-265A. X-ray crystal structure analysis can also be used to identify the amino acids within a polypeptide with which an antibody interacts.

The present invention includes LEPRxGP130 bispecific antigen-binding molecules comprising a D1 (GP130-binding) domain that binds to the same epitope as any of the specific exemplary anti-GP130 antibodies or antigen-binding domains described herein (e.g. antibodies comprising any of the amino acid sequences as set forth in Table 1 herein). The present invention includes LEPRxGP130 bispecific antigen-binding molecules comprising a D2 (LEPR-binding) domain that binds to the same epitope as any of the specific exemplary anti-LEPR antibodies or antigen-binding domains described herein (e.g. antibodies comprising any of the amino acid sequences as set forth in Table 18 herein). Likewise, the present invention also includes LEPRxGP130 bispecific antigen-binding molecules comprising a D1 (GP130-binding) domain that competes for binding to GP130 with any of the specific exemplary anti-GP130 antibodies described herein (e.g. antibodies comprising any of the amino acid sequences as set forth in Table 1 herein). Moreover, the present invention also includes LEPRxGP130 bispecific antigen-binding molecules comprising a D2 (LEPR-binding) domain that competes for binding to LEPR with any of the specific exemplary anti-LEPR antibodies described herein (e.g. antibodies comprising any of the amino acid sequences as set forth in Table 18 herein).

One can easily determine whether an antibody or antigen-binding domain binds to the same epitope as, or competes for binding with, a reference anti-GP130 or anti-LEPR antibody by using routine methods known in the art and exemplified herein. For example, to determine if a test antibody binds to the same epitope as a reference anti-GP130 or anti-LEPR antibody of the invention, the reference antibody is allowed to bind to a target molecule (i.e., GP130 or LEPR protein, as the case may be). Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody of the invention. Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, Biacore, flow cytometry or any other quantitative or qualitative antibody-binding assay available in the art. In accordance with certain embodiments of the present invention, two antibodies bind to the same (or overlapping) epitope if, e.g., a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990:50:1495-1502). Alternatively, two antibodies are deemed to bind to the same epitope if essentially all amino acid mutations in the antigen that reduce or eliminate binding of one antibody reduce or eliminate binding of the other. Two antibodies are deemed to have “overlapping epitopes” if only a subset of the amino acid mutations that reduce or eliminate binding of one antibody reduce or eliminate binding of the other.

To determine if an antibody competes for binding (or cross-competes for binding) with a reference anti-GP130 or anti-LEPR antibody, the above-described binding methodology is performed in two orientations: In a first orientation, the reference antibody is allowed to bind to the target molecule under saturating conditions followed by assessment of binding of the test antibody to the target molecule. In a second orientation, the test antibody is allowed to bind to a target molecule under saturating conditions followed by assessment of binding of the reference antibody to the target molecule. If, in both orientations, only the first (saturating) antibody is capable of binding to the target molecule, then it is concluded that the test antibody and the reference antibody compete for binding to the target molecule. As will be appreciated by a person of ordinary skill in the art, an antibody that competes for binding with a reference antibody may not necessarily bind to the same epitope as the reference antibody, but may sterically block binding of the reference antibody by binding an overlapping or adjacent epitope.

The antigen-binding domains (D1 and/or D2) of the bispecific antigen-binding molecules of the present invention may be described in terms of the domains of GP130 or LEPR with which the antigen-binding domain interacts. GP130 and LEPR proteins comprise various domains referred to as D1, D2, D3 and FNIII. Accordingly, the D1 and D2 antigen-binding domains of the bispecific antigen-binding molecules of the present invention, may bind a domain of LEPR or GP130 selected from the group consisting of D1, D2, D3, or FNIII. According to certain exemplary embodiments, LEPRxGP130 bispecific antigen binding molecules are provided wherein the D1 (anti-GP130) antigen-binding domain binds to the FNIII domain of GP130, and the D2 (anti-LEPR) antigen-binding domain binds to the FNIII domain of LEPR. Other binding domain combinations are contemplated within the scope of the present invention.

Preparation of Human Antibodies

The anti-GP130, anti-LEPR antibodies, and LEPRxGP130 bispecific antibodies of the present invention can be fully human antibodies. Methods for generating monoclonal antibodies, including fully human monoclonal antibodies are known in the art. Any such known methods can be used in the context of the present invention to make human antibodies that specifically bind to human GP130 and/or human LEPR.

Using VELOCIMMUNE™ technology, for example, or any other similar known method for generating fully human monoclonal antibodies, high affinity chimeric antibodies to human GP130 and/or human LEPR are initially isolated having a human variable region and a mouse constant region. As in the experimental section below, the antibodies are characterized and selected for desirable characteristics, including affinity, ligand blocking activity, selectivity, epitope, etc. If necessary, mouse constant regions are replaced with a desired human constant region, for example wild-type or modified IgG1 or IgG4, to generate fully human anti-GP130 and/or anti-LEPR antibodies. While the constant region selected may vary according to specific use, high affinity antigen-binding and target specificity characteristics reside in the variable region. In certain instances, fully human anti-GP130 and/or anti-LEPR antibodies are isolated directly from antigen-positive B cells.

Bioequivalents

The present invention includes variant anti-GP130, anti-LEPR antibodies, and LEPRxGP130 bispecific antibodies having amino acid sequences that vary from those of the described antibodies but that retain the ability to bind the relevant target antigen(s) (GP130 and/or LEPR) and exert one or more of the biological function(s) of the parent antibodies from which such variants are derived. Such variant antibodies and antibody fragments comprise one or more additions, deletions, or substitutions of amino acids when compared to parent sequence, but exhibit biological activity that is essentially equivalent to that of the described antibodies. Likewise, the present invention includes DNA sequences encoding anti-GP130, anti-LEPR antibodies, and LEPRxGP130 bispecific antibodies of the present invention, wherein such DNA sequences comprise one or more additions, deletions, or substitutions of nucleotides when compared to the disclosed parental sequence, but that encode anti-GP130, anti-LEPR antibodies, and LEPRxGP130 bispecific antibodies that are essentially bioequivalent to the exemplary antibodies disclosed herein. Examples of such variant amino acid and DNA sequences are discussed elsewhere herein.

Two antigen-binding proteins, or antibodies, are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding proteins are bioequivalent if there are no clinically meaningful differences in their safety, purity, and potency.

In one embodiment, two antigen-binding proteins are bioequivalent if a patient can be switched one or more times between the reference product and the biological product without an expected increase in the risk of adverse effects, including a clinically significant change in immunogenicity, or diminished effectiveness, as compared to continued therapy without such switching.

In one embodiment, two antigen-binding proteins are bioequivalent if they both act by a common mechanism or mechanisms of action for the condition or conditions of use, to the extent that such mechanisms are known.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Species Selectivity and Species Cross-Reactivity

The present invention, according to certain embodiments, provides anti-GP130, anti-LEPR antibodies, and LEPRxGP130 bispecific antibodies (and other antigen-binding molecules comprising anti-GP130 and/or anti-LEPR antigen-binding domains) that bind to human GP130 and human LEPR but not to the corresponding proteins from other species. The present invention also includes anti-GP130, anti-LEPR antibodies, and LEPRxGP130 bispecific antibodies (and antigen-binding molecules comprising anti-GP130 and/or anti-LEPR antigen-binding domains) that bind to human GP130 and human LEPR and to GP130 and LEPR from one or more non-human species. For example, the present invention includes bispecific antigen-binding molecules comprising a first and second antigen-binding domain, wherein the first antigen binding domain binds human and monkey (e.g., Macaca fascicularis) GP130 but does not bind rodent (rat and/or mouse) GP130. The present invention includes bispecific antigen-binding molecules comprising a first and second antigen-binding domain, wherein the second antigen binding domain binds human and monkey (e.g., Macaca fascicularis) LEPR but does not bind rodent (rat and/or mouse) LEPR.

The present invention further provides anti-GP130, anti-LEPR antibodies, and LEPRxGP130 bispecific antibodies (and other antigen-binding molecules comprising anti-GP130 and/or anti-LEPR antigen-binding domains) that bind to human GP130 and/or human LEPR, and may bind or not bind, as the case may be, to one or more of mouse, rat, guinea pig, hamster, gerbil, pig, cat, dog, rabbit, goat, sheep, cow, horse, camel, cynomologous, marmoset, rhesus or chimpanzee versions of the corresponding GP130 and/or LEPR proteins.

Therapeutic Formulation and Administration

The invention provides pharmaceutical compositions comprising anti-GP130, anti-LEPR antibodies, and LEPRxGP130 bispecific antibodies (and other antigen-binding molecules comprising anti-GP130 and/or anti-LEPR antigen-binding domains) of the present invention. The pharmaceutical compositions of the invention may be formulated with suitable carriers, excipients, and other agents that provide improved transfer, delivery, tolerance, and the like.

Therapeutic Uses of the Antibodies

The present invention includes methods comprising administering to a subject in need thereof (e.g., a mammal such as a human) a therapeutic composition comprising a LEPRxGP130 bispecific antigen-binding molecule (e.g., a LEPRxGP130 bispecific antigen-binding molecule comprising any of the D1 and D2 components as set forth in Table 20 herein). The therapeutic composition can comprise any of the LEPRxGP130 bispecific antigen-binding molecules disclosed herein, and a pharmaceutically acceptable carrier or diluent.

The LEPRxGP130 bispecific antigen-binding molecules of the invention are useful, inter alia, for the treatment, prevention and/or amelioration of any disease or disorder associated with or mediated by leptin deficiency, leptin resistance, hypoleptinemia, or otherwise treatable by stimulating or activating LEPR signaling or mimicking the natural activity of leptin in vitro or in vivo. For example, the bispecific antigen-binding molecules of the present invention are useful for treating lipodystrophy conditions. Exemplary lipodystrophy conditions that are treatable by the bispecific antigen-binding molecules of the present invention include, e.g., congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy, and HIV-associated lipodystrophy.

The LEPRxGP130 bispecific antigen-binding molecules of the present invention are also useful for the treatment or prevention of one or more diseases or disorders selected from the group consisting of obesity, metabolic syndrome, diet-induced food craving, functional hypothalamic amenorrhea, type 1 diabetes, type 2 diabetes, insulin resistance, severe insulin resistance including severe insulin resistance due to mutation in insulin receptor, severe insulin resistance not caused by mutation in the insulin receptor, severe insulin resistance caused by a mutation in downstream signaling pathways or induced by other causes, non-alcoholic and alcoholic fatty liver diseases, Alzheimer's disease, leptin deficiency, leptin resistance, lipodystrophies, Leprechaunism/Donohue syndrome, Rabson-Mendenhall syndrome.

In the context of the methods of treatment described herein, the LEPRxGP130 bispecific antigen-binding molecule may be administered as a monotherapy (i.e., as the only therapeutic agent) or in combination with one or more additional therapeutic agents (examples of which are described elsewhere herein).

Combination Therapies and Formulations

The present invention includes compositions and therapeutic formulations comprising any of the LEPRxGP130 bispecific antigen-binding molecules described herein in combination with one or more additional therapeutically active components, and methods of treatment comprising administering such combinations to subjects in need thereof.

The LEPRxGP130 bispecific antigen-binding molecules of the present invention may be co-formulated with and/or administered in combination with one or more additional therapeutically active component(s), such as. e.g., pharmaceutical products prescribed for the treatment of obesity, hypercholesterolemia, hyperlipidemia, type 2 diabetes, type 1 diabetes, appetite control, infertility, etc. Examples of such additional therapeutically active components include, e.g., recombinant human leptin (e.g., metreleptin [MYALEPT]), PCSK9 inhibitors (e.g., anti-PCSK9 antibodies [alirocumab, evolocumab, bococizumab, lodelcizumab, ralpancizumab, etc.]), statins (atorvastatin, rosuvastatin, cerivastatin, pitavastatin, fluvastatin, simvastatin, lovastatin, pravastatin, etc.), ezetimibe, insulin, insulin variants, insulin secretagogues, metformin, sulfonylureas, sodium glucose cotransporter 2 (SGLT2) Inhibitors (e.g., dapaglifozin, canaglifozin, empagliflozin, etc.), GLP-1 agonists/analogues (e.g., extendin-4, exenatide, liraglutide, lixisenatide, albiglutide, dulaglutide, etc.), glucagon (GCG) inhibitors (e.g., anti-GCG antibodies), glucagon receptor (GCGR) inhibitors (e.g., anti-GCGR antibodies, small molecule GCGR antagonists, GCGR-specific antisense oligonucleotides, anti-GCGR aptamers [e.g., Spiegelmers], etc.), angiopoietin-like protein (ANGPTL) inhibitors (e.g., anti-ANGPTL3 antibodies, anti-ANGPTL4 antibodies, anti-ANGPTL8 antibodies, etc.), Phentermine, Orlistat, Topiramate, Bupropion, Topiramate/Phentermine, Bupropion/Naltrexone, Bupropion/Zonisamide, Pramlintide/Metrelepin, Lorcaserin, Cetilistat, Tesofensine, Velneperit, etc.

The additional therapeutically active component(s), e.g., any of the agents listed above or derivatives thereof, may be administered just prior to, concurrent with, or shortly after the administration of a LEPRxGP130 bispecific antigen-binding molecule of the present invention; (for purposes of the present disclosure, such administration regimens are considered the administration of a LEPRxGP130 bispecific antigen-binding molecule “in combination with” an additional therapeutically active component). The present invention includes pharmaceutical compositions in which a LEPRxGP130 bispecific antigen-binding molecule of the present invention is co-formulated with one or more of the additional therapeutically active component(s) as described elsewhere herein.

Administration Regimens

According to certain embodiments of the present invention, multiple doses of a LEPRxGP130 bispecific antigen-binding molecule (or a pharmaceutical composition comprising a combination of a LEPRxGP130 bispecific antigen-binding molecule and any of the additional therapeutically active agents mentioned herein) may be administered to a subject over a defined time course. The methods according to this aspect of the invention comprise sequentially administering to a subject multiple doses of a LEPRxGP130 bispecific antigen-binding molecule of the invention. As used herein, “sequentially administering” means that each dose of LEPRxGP130 bispecific antigen-binding molecule is administered to the subject at a different point in time, e.g., on different days separated by a predetermined interval (e.g., hours, days, weeks or months). The present invention includes methods which comprise sequentially administering to the patient a single initial dose of a LEPRxGP130 bispecific antigen-binding molecule, followed by one or more secondary doses of the LEPRxGP130 bispecific antigen-binding molecule, and optionally followed by one or more tertiary doses of the LEPRxGP130 bispecific antigen-binding molecule.

The terms “initial dose,” “secondary doses,” and “tertiary doses,” refer to the temporal sequence of administration of the LEPRxGP130 bispecific antigen-binding molecule of the invention. Thus, the “initial dose” is the dose which is administered at the beginning of the treatment regimen (also referred to as the “baseline dose,” “loading dose,” “starting dose,” and the like); the “secondary doses” are the doses which are administered after the initial dose; and the “tertiary doses” are the doses which are administered after the secondary doses. The initial, secondary, and tertiary doses may all contain the same amount of LEPRxGP130 bispecific antigen-binding molecule, but generally may differ from one another in terms of frequency of administration. In certain embodiments, however, the amount of LEPRxGP130 bispecific antigen-binding molecule contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

### Devices

The present invention also provides a vessel (e.g., a vial or chromatography column) or injection device (e.g., syringe, pre-filled syringe or autoinjector) comprising a bispecific antigen binding molecule (e.g., pharmaceutical formulation thereof) set forth herein. The vessel or injection device may be packaged into a kit.

An injection device is a device that introduces a substance into the body of a subject (e.g., a human) via a parenteral route, e.g., intraocular, intravitreal, intramuscular, subcutaneous or intravenous. For example, an injection device may be a syringe (e.g., pre-filled with the pharmaceutical formulation, such as an autoinjector) which, for example, includes a cylinder or barrel for holding fluid to be injected (e.g., comprising the antibody or fragment or a pharmaceutical formulation thereof), a needle for piecing skin, blood vessels or other tissue for injection of the fluid; and a plunger for pushing the fluid out of the cylinder and through the needle bore and into the body of the subject.

The present invention includes methods for administering a bispecific antigen binding molecule of the present invention comprising introducing e.g., injecting, the molecule into the body of the subject, e.g., with an injection device.

### Expression Methods

The present invention includes recombinant methods for making a bispecific antigen binding molecule of the present invention, or an immunoglobulin chain thereof, comprising (i) introducing, into a host cell, one or more polynucleotides encoding light and/or heavy immunoglobulin chains of such a bispecific antigen binding molecule, for example, wherein the polynucleotide is in a vector; and/or integrates into the host cell chromosome and/or is operably linked to a promoter; (ii) culturing the host cell (e.g., mammalian, fungal, Chinese hamster ovary (CHO), Pichia or Pichia pastoris) under conditions favorable to expression of the polynucleotide and, (iii) optionally, isolating the bispecific antigen binding molecule or immunoglobulin chain from the host cell and/or medium in which the host cell is grown. The product of such a method also forms part of the present invention along with a pharmaceutical composition thereof.

In an embodiment of the invention, a method for making a bispecific antigen binding molecule includes a method of purifying the molecule, e.g., by column chromatography, precipitation and/or filtration. The product of such a method also forms part of the present invention along with a pharmaceutical composition thereof.

Host cells comprising a bispecific antigen binding molecule of the present invention and/or a polynucleotide encoding immunoglobulin chains of such a molecule (e.g., in a vector) are also part of the present invention. Host cells include, for example, mammalian cells such as Chinese hamster ovary (CHO) cells and fungal cells such as Pichia cells (e.g., P.pastoris).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1. Generation of Anti-GP130 Antibodies

Anti-GP130 antibodies were obtained by immunizing a genetically engineered mouse comprising DNA encoding human immunoglobulin heavy and kappa light chain variable regions with an immunogen comprising recombinant human GP130 extracellular domain. The mice used for the immunizations express a “universal light chain.” That is, the antibodies produced in this mouse have different heavy chain variable regions but essentially identical light chain variable domains.

The antibody immune response was monitored by a GP130-specific immunoassay. When a desired immune response was achieved splenocytes were harvested and fused with mouse myeloma cells to preserve their viability and from hybridoma cell lines. The hybridoma cell lines were screened and selected to identify cell lines that produce GP130-specific antibodies. Using this technique several anti-GP130 chimeric antibodies (i.e., antibodies possessing human variable domains and mouse constant domains) were obtained. In addition, several fully human anti-GP130 antibodies were isolated directly from antigen-positive B cells without fusion to myeloma cells, as described in US 2007/0280945A1.

Certain biological properties of the exemplary anti-GP130 antibodies generated in accordance with the methods of this Example, and bispecific antibodies constructed therefrom, are described in detail in the Examples set forth below.

### Example 2. Heavy and Light Chain Variable Region Amino Acid and Nucleic Acid Sequences of Anti-GP130

Table 1 sets forth the amino acid sequence identifiers of the heavy and light chain variable regions and CDRs of selected anti-LEPR antibodies of the invention. (As noted above, all antibodies generated in Example 1 possess the same light chain variable region and the same light chain CDR sequences as well). The corresponding nucleic acid sequence identifiers are set forth in Table 2.

TABLE 1
Anti-GP130 Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| MAb16614 | 18 | 20 | 22 | 24 | 10 | 12 | 14 | 16 |
| MAb16618 | 26 | 28 | 30 | 32 | 10 | 12 | 14 | 16 |
| MAb16622 | 34 | 36 | 38 | 40 | 10 | 12 | 14 | 16 |
| MAb16623 | 42 | 44 | 46 | 48 | 10 | 12 | 14 | 16 |
| MAb16636 | 50 | 52 | 54 | 56 | 10 | 12 | 14 | 16 |
| MAb16637 | 58 | 60 | 62 | 64 | 10 | 12 | 14 | 16 |
| MAb16656 | 66 | 68 | 70 | 72 | 10 | 12 | 14 | 16 |
| MAb16659 | 74 | 76 | 78 | 80 | 10 | 12 | 14 | 16 |
| MAb16662 | 82 | 84 | 86 | 88 | 10 | 12 | 14 | 16 |
| MAb16664 | 90 | 92 | 94 | 96 | 10 | 12 | 14 | 16 |
| MAb16665 | 98 | 100 | 102 | 104 | 10 | 12 | 14 | 16 |
| MAb16666 | 106 | 108 | 110 | 112 | 10 | 12 | 14 | 16 |
| MAb16669 | 114 | 116 | 118 | 120 | 10 | 12 | 14 | 16 |
| MAb16673 | 122 | 124 | 126 | 128 | 10 | 12 | 14 | 16 |
| MAb16676 | 130 | 132 | 134 | 136 | 10 | 12 | 14 | 16 |
| MAb16680 | 138 | 140 | 142 | 144 | 10 | 12 | 14 | 16 |
| MAb16682 | 146 | 148 | 150 | 152 | 10 | 12 | 14 | 16 |
| MAb16683 | 154 | 156 | 158 | 160 | 10 | 12 | 14 | 16 |

TABLE 2
Anti-GP130 Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| MAb16614 | 17 | 19 | 21 | 23 | 9 | 11 | 13 | 15 |
| MAb16618 | 25 | 27 | 29 | 31 | 9 | 11 | 13 | 15 |
| MAb16622 | 33 | 35 | 37 | 39 | 9 | 11 | 13 | 15 |
| MAb16623 | 41 | 43 | 45 | 47 | 9 | 11 | 13 | 15 |
| MAb16636 | 49 | 51 | 53 | 55 | 9 | 11 | 13 | 15 |
| MAb16637 | 57 | 59 | 61 | 63 | 9 | 11 | 13 | 15 |
| MAb16656 | 65 | 67 | 69 | 71 | 9 | 11 | 13 | 15 |
| MAb16659 | 73 | 75 | 77 | 79 | 9 | 11 | 13 | 15 |
| MAb16662 | 81 | 83 | 85 | 87 | 9 | 11 | 13 | 15 |
| MAb16664 | 89 | 91 | 93 | 95 | 9 | 11 | 13 | 15 |
| MAb16665 | 97 | 99 | 101 | 103 | 9 | 11 | 13 | 15 |
| MAb16666 | 105 | 107 | 109 | 111 | 9 | 11 | 13 | 15 |
| MAb16669 | 113 | 115 | 117 | 119 | 9 | 11 | 13 | 15 |
| MAb16673 | 121 | 123 | 125 | 127 | 9 | 11 | 13 | 15 |
| MAb16676 | 129 | 131 | 133 | 135 | 9 | 11 | 13 | 15 |
| MAb16680 | 137 | 139 | 141 | 143 | 9 | 11 | 13 | 15 |
| MAb16682 | 145 | 147 | 149 | 151 | 9 | 11 | 13 | 15 |
| MAb16683 | 153 | 155 | 157 | 159 | 9 | 11 | 13 | 15 |

The antibodies of the present invention can be of any isotype. For example, anti-GP130 antibodies of the invention may comprise variable domain and CDR sequences as set forth in Tables 1 and 2 and a human Fc domain of isotype IgG4, IgG1, etc. For certain applications or experiments the Fc domain may be a mouse Fc domain. As will be appreciated by a person of ordinary skill in the art, an antibody having a particular Fc isotype can be converted to an antibody with a different Fc isotype (e.g., an antibody with a mouse IgG4 Fc can be converted to an antibody with a human IgG1, etc.), but in any event, the variable domains (including the CDRs)—which are indicated by the numerical identifiers shown in Tables 1 and 2—will remain the same, and the binding properties are expected to be identical or substantially similar regardless of the nature of the Fc domain.

### Example 3. Biacore Binding Kinetics of Anti-GP130 Monoclonal Antibodies Binding to Different GP130 Reagents Measured at 25° C. and 37° C.

Equilibrium dissociation constant (K_{D}) for different GP130 reagents binding to purified anti-GP130 monoclonal antibodies were determined using a real-time surface plasmon resonance based Biacore 4000 biosensor. All binding studies were performed in 10 mM HEPES, 150 mM NaCl, 3 mM EDTA, and 0.05% v/v Surfactant Tween-20, pH 7.4 (HBS-ET) running buffer at 25° C. and 37° C. The Biacore sensor surface was first derivatized by amine coupling with a monoclonal mouse anti-human Fc antibody (GE, #BR-1008-39) to capture anti-GP130 monoclonal antibodies. Binding studies were performed on the following monomeric and dimeric GP130 reagents: human GP130 extracellular domain expressed with a C-terminal myc-myc-hexahistidine tag (hGP130-mmH; SEQ ID NO:191), Macaca fascicularis GP130 extracellular domain expressed with a C-terminal myc-myc-hexahistidine tag (mfGP130-mmH; SEQ ID NO:194), human GP130 extracellular domain expressed with a C-terminal mouse IgG2a Fc tag (hGP130-hFc; SEQ ID NO:197), mouse GP130 extracellular domain expressed with a C-terminal myc-myc-hexahistidine tag (mGP130-mmH; SEQ ID NO:196) and rat GP130 extracellular domain expressed with a C-terminal myc-myc-hexahistidine tag (rGP130-mmH; SEQ ID NO:195). Reagents tagged with “mmH” are monomeric, whereas reagents tagged with “mFc” are dimeric. Thus, for example, “hGP130-mmH” is also referred to as “monomeric human GP130,” and “hGP130-mFc” is also referred to as “dimeric human GP130.”

Different concentrations of hGP130-mmH, mfGP130-mmH, hGP130-mFc (100 nM-3.7 nM; 3-fold serial dilution) or 100 nM of mGP130-mmH and rGP130-mmH were first prepared in HBS-ET running buffer and were injected over anti-human Fc captured anti-GP130 monoclonal antibody surface for 4 minutes at a flow rate of 304/minute, while the dissociation of monoclonal antibody bound GP130 reagent was monitored for 10 minutes in HBS-ET running buffer. The association rate (k a) and dissociation rate (k_{d}) were determined by fitting the real-time binding sensorgrams to a 1:1 binding model with mass transport limitation using Scrubber 2.0c curve-fitting software. Binding dissociation equilibrium constant (K_{D}) and dissociative half-life (t ½) were calculated from the kinetic rates as:

$${K}_{D} ( M ) = \frac{kd}{ka} , and t 1 / 2 ( min ) = \frac{ln ( 2 )}{60 * kd}$$

Binding kinetics parameters for hGP130-MMH, mfGP130-MMH, hGP130.mFc, mGP130-MMH or rGP130-MMH binding to different anti-GP130 monoclonal antibodies of the invention at 25° C. and 37° C. are shown in Tables 3 through 12.

TABLE 3
Binding kinetics parameters of hGP130-MMH binding
to GP130 monoclonal antibodies at 25° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 143 ± 0.67 | 18 | 5.52E+04 | 1.01E−02 | 1.83E−07 | 1.1 |
| MAb16618 | 182 ± 0.52 | 52 | 3.54E+05 | 4.25E−02 | 1.20E−07 | 0.3 |
| MAb16622 | 197 ± 0.95 | 36 | 1.20E+05 | 2.43E−02 | 2.03E−07 | 0.5 |
| MAb16623 | 243 ± 1.48 | 77 | 1.80E+05 | 3.77E−03 | 2.10E−08 | 3 |
| MAb16636 | 195 ± 0.45 | 37 | 2.70E+04 | 9.93E−04 | 3.68E−08 | 12 |
| MAb16637 | 306 ± 1.71 | 91 | 7.11E+04 | 1.10E−03 | 1.55E−08 | 10 |
| MAb16641 | 154 ± 0.93 | 111 | 3.56E+05 | 8.87E−04 | 2.49E−09 | 13 |
| MAb16646 | 167 ± 0.45 | 58 | 5.88E+04 | 7.73E−04 | 1.31E−08 | 15 |
| MAb16656 | 189 ± 0.44 | 44 | 3.59E+04 | 8.25E−04 | 2.30E−08 | 14 |
| MAb16659 | 212 ± 0.39 | 77 | 6.93E+05 | 4.59E−04 | 6.63E−10 | 25 |
| MAb16662 | 215 ± 0.67 | 22 | 2.35E+05 | 2.90E−02 | 1.23E−07 | 0.4 |
| MAb16664 | 217 ± 0.69 | 14 | 6.98E+05 | 1.64E−03 | 2.35E−09 | 7 |
| MAb16665 | 294 ± 1.48 | 20 | 1.15E+05 | 4.73E−02 | 4.11E−07 | 0.2 |
| MAb16666 | 317 ± 2.83 | 138 | 3.17E+05 | 4.56E−03 | 1.44E−08 | 3 |
| MAb16669 | 152 ± 0.5 | 17 | 1.14E+04 | 4.03E−04 | 3.54E−08 | 29 |
| MAb16673 | 158 ± 0.32 | 58 | 2.15E+05 | 1.41E−02 | 6.55E−08 | 0.8 |
| MAb16676 | 176 ± 0.33 | 51 | 2.87E+05 | 6.31E−04 | 2.19E−09 | 18 |
| MAb16680 | 270 ± 0.91 | 57 | 1.47E+05 | 4.75E−04 | 3.24E−09 | 24 |
| MAb16682 | 186 ± 0.55 | 21 | 1.21E+05 | 1.64E−02 | 1.36E−07 | 0.7 |
| MAb16683 | 238 ± 2.72 | 54 | 6.97E+04 | 3.25E−03 | 4.67E−08 | 4 |
| MAb16684 | 204 ± 1.09 | 40 | 5.32E+04 | 6.22E−03 | 1.17E−07 | 1.9 |
| MAb16687 | 172 ± 0.55 | 111 | 2.04E+05 | 4.86E−04 | 2.38E−09 | 24 |
| MAb16692 | 182 ± 1.08 | 121 | 2.40E+05 | 6.98E−04 | 2.91E−09 | 17 |
| MAb16693 | 159 ± 0.52 | 29 | 1.50E+05 | 1.51E−02 | 1.00E−07 | 0.8 |
| MAb16695 | 186 ± 0.79 | 122 | 2.80E+05 | 1.05E−03 | 3.77E−09 | 11 |
| MAb16702 | 208 ± 0.78 | 75 | 1.39E+05 | 3.74E−04 | 2.69E−09 | 31 |
| IgG4 Isotype | 229 ± 1.42 | 2 | NB | NB | NB | NB |
| Control | | | | | | |

NB indicates that no binding was observed under the current experimental conditions.

TABLE 4
Binding kinetics parameters of hGP130-MMH binding
to GP130 monoclonal antibodies at 37° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 176 ± 2.98 | 10 | 2.47E+05 | 3.00E−02 | 1.21E−07 | 0.4 |
| MAb16618 | 219 ± 2.04 | 36 | 1.08E+06 | 1.12E−01 | 1.03E−07 | 0.1 |
| MAb16622 | 238 ± 3.05 | 30 | 2.55E+05 | 5.74E−02 | 2.25E−07 | 0.2 |
| MAb16623 | 285 ± 3.24 | 68 | 3.81E+05 | 1.23E−02 | 3.22E−08 | 0.9 |
| MAb16636 | 231 ± 2.35 | 50 | 6.16E+04 | 4.41E−03 | 7.16E−08 | 2.6 |
| MAb16637 | 352 ± 2.31 | 110 | 1.97E+05 | 3.42E−03 | 1.73E−08 | 3 |
| MAb16641 | 188 ± 3.13 | 107 | 7.65E+05 | 2.49E−03 | 3.25E−09 | 5 |
| MAb16646 | 212 ± 2.83 | 66 | 9.81E+04 | 3.31E−03 | 3.38E−08 | 3.5 |
| MAb16656 | 214 ± 1.6 | 50 | 7.01E+04 | 4.26E−03 | 6.07E−08 | 2.7 |
| MAb16659 | 239 ± 1.56 | 82 | 9.28E+05 | 1.71E−03 | 1.84E−09 | 7 |
| MAb16662 | 245 ± 0.93 | 17 | 4.36E+05 | 5.85E−02 | 1.34E−07 | 0.2 |
| MAb16664 | 251 ± 2.08 | 16 | 5.80E+05 | 8.26E−03 | 1.42E−08 | 1.4 |
| MAb16665 | 336 ± 2.56 | 19 | 4.23E+05 | 6.93E−02 | 1.64E−07 | 0.2 |
| MAb16666 | 350 ± 4.52 | 104 | 5.88E+05 | 1.01E−02 | 1.71E−08 | 1.1 |
| MAb16669 | 173 ± 1.38 | 25 | 5.51E+04 | 1.06E−03 | 1.93E−08 | 11 |
| MAb16673 | 180 ± 1.31 | 40 | 5.36E+05 | 4.04E−02 | 7.54E−08 | 0.3 |
| MAb16676 | 192 ± 1.72 | 52 | 3.93E+05 | 2.36E−03 | 6.01E−09 | 5 |
| MAb16680 | 291 ± 1.59 | 58 | 9.48E+04 | 2.06E−03 | 2.17E−08 | 6 |
| MAb16682 | 211 ± 2.32 | 16 | 3.01E+05 | 3.41E−02 | 1.13E−07 | 0.3 |
| MAb16683 | 262 ± 1.88 | 38 | 1.07E+05 | 1.25E−02 | 1.17E−07 | 0.9 |
| MAb16684 | 220 ± 1.73 | 18 | 1.63E+05 | 3.60E−02 | 2.21E−07 | 0.3 |
| MAb16687 | 184 ± 1.85 | 107 | 3.22E+05 | 1.82E−03 | 5.66E−09 | 6 |
| MAb16692 | 197 ± 1.78 | 107 | 7.24E+05 | 3.05E−03 | 4.21E−09 | 4 |
| MAb16693 | 180 ± 1.26 | 22 | 3.82E+05 | 4.02E−02 | 1.05E−07 | 0.3 |
| MAb16695 | 192 ± 1.56 | 87 | 4.94E+05 | 6.27E−03 | 1.27E−08 | 1.8 |
| MAb16702 | 216 ± 0.72 | 68 | 2.35E+05 | 1.66E−03 | 7.05E−09 | 7 |
| IgG4 Isotype | 258 ± 1.09 | 2 | NB | NB | NB | NB |
| Control | | | | | | |

NB indicates that no binding was observed under the current experimental conditions.

At 25° C., anti-GP130 monoclonal antibodies bound to hGP130-MMH with K_{D} values ranging from 663 pM to 411 nM, as shown in Table 3. At 37° C., anti-GP130 monoclonal antibodies bound to hGP130-MMH with K_{D} values ranging from 1.84 nM to 225 nM, as shown in Table 4.

TABLE 5
Binding kinetics parameters of mfGP130-MMH binding
to GP130 monoclonal antibodies at 25° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 142 ± 0.65 | 16 | 7.44E+04 | 1.04E−02 | 1.39E−07 | 1.1 |
| MAb16618 | 181 ± 0.48 | 40 | 3.74E+05 | 6.21E−02 | 1.66E−07 | 0.2 |
| MAb16622 | 195 ± 0.74 | 34 | 1.32E+05 | 2.00E−02 | 1.51E−07 | 0.6 |
| MAb16623 | 240 ± 0.79 | 74 | 1.87E+05 | 4.76E−03 | 2.55E−08 | 2.4 |
| MAb16636 | 193 ± 0.49 | 23 | 2.01E+04 | 2.16E−03 | 1.07E−07 | 5 |
| MAb16637 | 304 ± 3.59 | 87 | 6.83E+04 | 1.09E−03 | 1.59E−08 | 11 |
| MAb16641 | 153 ± 3.55 | 109 | 3.44E+05 | 8.60E−04 | 2.50E−09 | 13 |
| MAb16646 | 166 ± 0.29 | 52 | 4.79E+04 | 7.72E−04 | 1.61E−08 | 15 |
| MAb16656 | 187 ± 0.26 | 42 | 4.44E+04 | 8.46E−04 | 1.90E−08 | 14 |
| MAb16659 | 211 ± 0.43 | 74 | 5.42E+05 | 4.40E−04 | 8.12E−10 | 26 |
| MAb16662 | 214 ± 1.92 | 15 | 2.07E+05 | 3.80E−02 | 1.84E−07 | 0.3 |
| MAb16664 | 216 ± 1.31 | 13 | 5.43E+05 | 1.75E−03 | 3.21E−09 | 7 |
| MAb16665 | 294 ± 1.19 | 16 | 2.17E+05 | 5.05E−02 | 2.33E−07 | 0.2 |
| MAb16666 | 317 ± 2.67 | 123 | 2.90E+05 | 5.30E−03 | 1.83E−08 | 2.2 |
| MAb16669 | 151 ± 0.6 | 3 | NB | NB | NB | NB |
| MAb16673 | 157 ± 0.25 | 86 | 3.12E+05 | 6.93E−03 | 2.22E−08 | 1.7 |
| MAb16676 | 175 ± 0.54 | 49 | 2.72E+05 | 5.97E−04 | 2.20E−09 | 19 |
| MAb16680 | 267 ± 0.84 | 52 | 1.20E+05 | 4.89E−04 | 4.07E−09 | 24 |
| MAb16682 | 184 ± 0.48 | 20 | 1.28E+05 | 1.68E−02 | 1.32E−07 | 0.7 |
| MAb16683 | 236 ± 1.97 | 59 | 6.57E+04 | 2.46E−03 | 3.74E−08 | 5 |
| MAb16684 | 203 ± 0.61 | 4 | IC | IC | IC | IC |
| MAb16687 | 170 ± 0.58 | 108 | 1.84E+05 | 5.24E−04 | 2.85E−09 | 22 |
| MAb16692 | 179 ± 0.96 | 109 | 2.09E+05 | 7.45E−04 | 3.56E−09 | 16 |
| MAb16693 | 159 ± 0.33 | 6 | NB | NB | NB | NB |
| MAb16695 | 184 ± 0.5 | 120 | 2.67E+05 | 9.98E−04 | 3.74E−09 | 12 |
| MAb16702 | 205 ± 0.74 | 48 | 5.63E+04 | 5.95E−04 | 1.06E−08 | 19 |
| IgG4 Isotype | 227 ± 2.09 | 2 | NB | NB | NB | NB |
| Control | | | | | | |

NB indicates that no binding was observed under the current experimental conditions.

TABLE 6
Binding kinetics parameters of mfGP130-MMH binding to GP130
monoclonal antibodies at 37° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 168 ± 1.52 | 8 | 3.70E+05 | 2.67E−02 | 7.22E−08 | 0.4 |
| MAb16618 | 210 ± 1.02 | 26 | 1.33E+06 | 1.20E−01 | 9.03E−08 | 0.1 |
| MAb16622 | 229 ± 2 | 28 | 2.72E+05 | 5.37E−02 | 1.98E−07 | 0.2 |
| MAb16623 | 274 ± 1.25 | 60 | 3.52E+05 | 1.56E−02 | 4.43E−08 | 0.7 |
| MAb16636 | 222 ± 1.09 | 25 | 3.95E+04 | 9.45E−03 | 2.39E−07 | 1.2 |
| MAb16637 | 342 ± 1.61 | 104 | 1.78E+05 | 2.63E−03 | 1.48E−08 | 4 |
| MAb16641 | 179 ± 1.7 | 98 | 6.94E+05 | 2.42E−03 | 3.48E−09 | 5 |
| MAb16646 | 202 ± 1.18 | 58 | 8.64E+04 | 3.37E−03 | 3.91E−08 | 3.4 |
| MAb16656 | 208 ± 1.42 | 47 | 6.70E+04 | 4.11E−03 | 6.13E−08 | 2.8 |
| MAb16659 | 232 ± 1.18 | 78 | 7.47E+05 | 1.74E−03 | 2.34E−09 | 7 |
| MAb16662 | 238 ± 0.85 | 14 | 3.55E+05 | 5.92E−02 | 1.67E−07 | 0.2 |
| MAb16664 | 243 ± 1.68 | 16 | 6.80E+05 | 7.88E−03 | 1.16E−08 | 1.5 |
| MAb16665 | 330 ± 0.88 | 14 | 4.92E+05 | 1.01E−01 | 2.05E−07 | 0.1 |
| MAb16666 | 340 ± 3.16 | 90 | 5.28E+05 | 1.31E−02 | 2.47E−08 | 0.9 |
| MAb16669 | 167 ± 0.83 | 8 | NB | NB | NB | NB |
| MAb16673 | 175 ± 1.1 | 64 | 6.17E+05 | 2.11E−02 | 3.41E−08 | 0.5 |
| MAb16676 | 187 ± 1.43 | 48 | 3.41E+05 | 2.43E−03 | 7.12E−09 | 5 |
| MAb16680 | 284 ± 1.39 | 51 | 9.07E+04 | 2.17E−03 | 2.39E−08 | 5 |
| MAb16682 | 205 ± 0.85 | 15 | 3.11E+05 | 3.86E−02 | 1.24E−07 | 0.3 |
| MAb16683 | 256 ± 1.73 | 43 | 9.65E+04 | 1.21E−02 | 1.25E−07 | 1.0 |
| MAb16684 | 214 ± 1.12 | 2 | NB | NB | NB | NB |
| MAb16687 | 179 ± 1.38 | 101 | 2.84E+05 | 1.97E−03 | 6.93E−09 | 6 |
| MAb16692 | 191 ± 1.17 | 97 | 6.39E+05 | 2.97E−03 | 4.65E−09 | 4 |
| MAb16693 | 175 ± 0.86 | 8 | NB | NB | NB | NB |
| MAb16695 | 186 ± 1.27 | 84 | 4.33E+05 | 5.92E−03 | 1.37E−08 | 2.0 |
| MAb16702 | 211 ± 0.9 | 46 | 8.66E+04 | 2.19E−03 | 2.52E−08 | 5 |
| IgG4 Isotype | 253 ± 1.16 | 3 | NB | NB | NB | NB |
| Control | | | | | | |

NB indicates that no binding was observed under the current experimental conditions.

23 out of 26 anti-GP130 monoclonal antibodies of the invention bound to mfGP130-MMH. At 25° C., anti-GP130 monoclonal antibodies bound to mfGP130-MMH with K_{D} values ranging from 812 pM to 233 nM, as shown in Table 5. At 37° C., anti-GP130 monoclonal antibodies bound to mfGP130-MMH with K_{D} values ranging from 2.34 nM to 239 nM, as shown in Table 6.

TABLE 7
Binding kinetics parameters of hGP130-mFc binding
to GP130 monoclonal antibodies at 25° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 143 ± 0.19 | 83 | 8.57E+04 | 3.83E−04 | 4.47E−09 | 30 |
| MAb16618 | 181 ± 0.44 | 202 | 1.19E+06 | 2.79E−04 | 2.36E−10 | 41 |
| MAb16622 | 196 ± 0.81 | 164 | 4.97E+05 | 3.06E−04 | 6.15E−10 | 38 |
| MAb16623 | 242 ± 1.53 | 184 | 4.44E+05 | 2.08E−04 | 4.68E−10 | 56 |
| MAb16636 | 194 ± 0.62 | 91 | 5.88E+04 | 1.13E−04 | 1.92E−09 | 102 |
| MAb16637 | 306 ± 1.48 | 189 | 2.32E+05 | 1.50E−04 | 6.47E−10 | 77 |
| MAb16641 | 153 ± 1.08 | 142 | 7.70E+05 | 2.97E−04 | 3.86E−10 | 39 |
| MAb16646 | 167 ± 0.41 | 77 | 7.19E+04 | 2.25E−04 | 3.13E−09 | 51 |
| MAb16656 | 188 ± 0.42 | 98 | 7.81E+04 | 1.32E−04 | 1.68E−09 | 88 |
| MAb16659 | 212 ± 0.34 | 186 | 1.72E+06 | 6.28E−05 | 3.66E−11 | 184 |
| MAb16662 | 215 ± 1.08 | 90 | 6.20E+05 | 4.79E−04 | 7.72E−10 | 24 |
| MAb16664 | 217 ± 0.62 | 26 | 3.05E+06 | 3.54E−04 | 1.16E−10 | 33 |
| MAb16665 | 293 ± 1.36 | 158 | 3.99E+05 | 1.09E−03 | 2.72E−09 | 11 |
| MAb16666 | 317 ± 2.14 | 253 | 1.14E+06 | 1.37E−04 | 1.20E−10 | 85 |
| MAb16669 | 152 ± 0.46 | 40 | 2.93E+04 | 2.41E−04 | 8.21E−09 | 48 |
| MAb16673 | 158 ± 0.38 | 169 | 5.60E+05 | 9.66E−05 | 1.73E−10 | 120 |
| MAb16676 | 176 ± 0.22 | 119 | 8.53E+05 | 7.13E−05 | 8.36E−11 | 162 |
| MAb16680 | 269 ± 0.69 | 79 | 1.72E+05 | 2.31E−04 | 1.34E−09 | 50 |
| MAb16682 | 186 ± 0.74 | 126 | 1.72E+05 | 3.61E−04 | 2.10E−09 | 32 |
| MAb16683 | 236 ± 0.73 | 107 | 1.67E+05 | 3.11E−04 | 1.86E−09 | 37 |
| MAb16684 | 204 ± 0.27 | 96 | 1.28E+05 | 4.59E−04 | 3.60E−09 | 25 |
| MAb16687 | 171 ± 0.64 | 178 | 4.44E+05 | 4.21E−05 | 9.50E−11 | 274 |
| MAb16692 | 180 ± 0.69 | 174 | 6.13E+05 | 1.18E−04 | 1.93E−10 | 98 |
| MAb16693 | 159 ± 0.41 | 123 | 2.09E+05 | 2.51E−04 | 1.20E−09 | 46 |
| MAb16695 | 185 ± 0.29 | 189 | 6.83E+05 | 2.06E−04 | 3.01E−10 | 56 |
| MAb16702 | 206 ± 1.11 | 159 | 6.22E+05 | 2.52E−04 | 4.05E−10 | 46 |
| IgG4 Isotype | 228 ± 1.43 | 5 | NB | NB | NB | NB |
| Control | | | | | | |

NB indicates that no binding was observed under the current experimental conditions

TABLE 8
Binding kinetics parameters of hGP130-mFc binding
to GP130 monoclonal antibodies at 37° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 172 ± 1.78 | 85 | 1.80E+05 | 1.72E−03 | 9.56E−09 | 7 |
| MAb16618 | 214 ± 1.83 | 236 | 1.39E+06 | 9.34E−04 | 6.74E−10 | 12 |
| MAb16622 | 232 ± 1.75 | 201 | 6.39E+05 | 8.25E−04 | 1.29E−09 | 14 |
| MAb16623 | 280 ± 2 | 224 | 5.80E+05 | 3.25E−04 | 5.60E−10 | 36 |
| MAb16636 | 225 ± 2.06 | 136 | 1.39E+05 | 1.67E−04 | 1.20E−09 | 69 |
| MAb16637 | 349 ± 2.73 | 246 | 4.96E+05 | 3.23E−04 | 6.50E−10 | 36 |
| MAb16641 | 184 ± 1.98 | 151 | 1.60E+06 | 8.28E−04 | 5.18E−10 | 14 |
| MAb16646 | 207 ± 1.7 | 99 | 1.59E+05 | 6.56E−04 | 4.14E−09 | 18 |
| MAb16656 | 212 ± 0.97 | 123 | 1.75E+05 | 2.97E−04 | 1.70E−09 | 39 |
| MAb16659 | 235 ± 1.2 | 227 | 1.91E+06 | 4.76E−05 | 2.49E−11 | 243 |
| MAb16662 | 240 ± 1.17 | 114 | 6.39E+05 | 2.65E−03 | 4.15E−09 | 4 |
| MAb16664 | 248 ± 1.36 | 37 | 1.33E+06 | 1.09E−04 | 8.15E−11 | 106 |
| MAb16665 | 334 ± 3.31 | 172 | 6.53E+05 | 2.56E−03 | 3.92E−09 | 5 |
| MAb16666 | 347 ± 3.54 | 285 | 1.21E+06 | 3.60E−04 | 2.98E−10 | 32 |
| MAb16669 | 170 ± 1.24 | 59 | 6.51E+04 | 3.80E−04 | 5.85E−09 | 30 |
| MAb16673 | 178 ± 1.09 | 199 | 6.84E+05 | 3.13E−04 | 4.57E−10 | 37 |
| MAb16676 | 190 ± 0.61 | 140 | 1.67E+06 | 9.45E−05 | 5.66E−11 | 122 |
| MAb16680 | 288 ± 0.8 | 81 | 2.43E+05 | 4.89E−04 | 2.01E−09 | 24 |
| MAb16682 | 207 ± 1.28 | 130 | 2.36E+05 | 1.96E−03 | 8.33E−09 | 6 |
| MAb16683 | 260 ± 1.27 | 111 | 1.96E+05 | 1.14E−03 | 5.80E−09 | 10 |
| MAb16684 | 217 ± 0.75 | 103 | 2.02E+05 | 9.96E−04 | 4.94E−09 | 12 |
| MAb16687 | 183 ± 1.22 | 193 | 5.89E+05 | 1.00E−05^{#} | 1.70E−11 | 1155^{#} |
| MAb16692 | 195 ± 0.86 | 196 | 1.30E+06 | 3.29E−04 | 2.52E−10 | 35 |
| MAb16693 | 178 ± 1.04 | 140 | 4.35E+05 | 1.25E−03 | 2.88E−09 | 9 |
| MAb16695 | 189 ± 0.93 | 183 | 1.32E+06 | 7.63E−04 | 5.80E−10 | 15 |
| MAb16702 | 214 ± 0.89 | 139 | 1.29E+06 | 7.35E−04 | 5.70E−10 | 16 |
| IgG4 Isotype | 256 ± 0.83 | 6 | NB | NB | NB | NB |
| Control | | | | | | |

NB indicates that no binding was observed under the current experimental conditions.
^{#}means no dissociation of hGP130-mFc from captured GP130 monoclonal antibody was observed and the k_{d} value was manually fixed at 1.00E−05 during the analysis.

At 25° C., anti-GP130 monoclonal antibodies bound to hGP130-mFc with K_{D} values ranging from 36.6 pM to 8.21 nM, as shown in Table 7. At 37° C., anti-GP130 monoclonal antibodies bound to hGP130-mFc with K_{D} values ranging from 17 pM to 9.56 nM, as shown in Table 8.

TABLE 9
Binding kinetics parameters of mGP130-MMH binding
to GP130 monoclonal antibodies at 25° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 142 | 1 | NB | NB | NB | NB |
| MAb16618 | 181 | 1 | NB | NB | NB | NB |
| MAb16622 | 195 | 1 | NB | NB | NB | NB |
| MAb16623 | 240 | 0 | NB | NB | NB | NB |
| MAb16636 | 194 | 0 | NB | NB | NB | NB |
| MAb16637 | 304 | 1 | NB | NB | NB | NB |
| MAb16641 | 152 | −1 | NB | NB | NB | NB |
| MAb16646 | 167 | −1 | NB | NB | NB | NB |
| MAb16656 | 187 | 0 | NB | NB | NB | NB |
| MAb16659 | 210 | 1 | NB | NB | NB | NB |
| MAb16662 | 213 | −1 | NB | NB | NB | NB |
| MAb16664 | 216 | 0 | NB | NB | NB | NB |
| MAb16665 | 295 | 0 | NB | NB | NB | NB |
| MAb16666 | 315 | 1 | NB | NB | NB | NB |
| MAb16669 | 151 | 0 | NB | NB | NB | NB |
| MAb16673 | 157 | 0 | NB | NB | NB | NB |
| MAb16676 | 175 | 0 | NB | NB | NB | NB |
| MAb16680 | 267 | 0 | NB | NB | NB | NB |
| MAb16682 | 184 | 1 | NB | NB | NB | NB |
| MAb16683 | 235 | 0 | NB | NB | NB | NB |
| MAb16684 | 202 | 0 | NB | NB | NB | NB |
| MAb16687 | 170 | 1 | NB | NB | NB | NB |
| MAb16692 | 179 | −1 | NB | NB | NB | NB |
| MAb16693 | 158 | −1 | NB | NB | NB | NB |
| MAb16695 | 183 | 0 | NB | NB | NB | NB |
| MAb16702 | 206 | 0 | NB | NB | NB | NB |
| IgG4 Isotype | 225 | 0 | NB | NB | NB | NB |
| Control | | | | | | |

NB indicates that no binding was observed under the current experimental conditions.

TABLE 10
Binding kinetics parameters of mGP130-MMH binding
to GP130 monoclonal antibodies at 37° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 167 | 0 | NB | NB | NB | NB |
| MAb16618 | 209 | 0 | NB | NB | NB | NB |
| MAb16622 | 227 | 1 | NB | NB | NB | NB |
| MAb16623 | 272 | 0 | NB | NB | NB | NB |
| MAb16636 | 220 | 0 | NB | NB | NB | NB |
| MAb16637 | 343 | −1 | NB | NB | NB | NB |
| MAb16641 | 178 | 0 | NB | NB | NB | NB |
| MAb16646 | 200 | 1 | NB | NB | NB | NB |
| MAb16656 | 208 | 0 | NB | NB | NB | NB |
| MAb16659 | 230 | 1 | NB | NB | NB | NB |
| MAb16662 | 235 | 1 | NB | NB | NB | NB |
| MAb16664 | 246 | 1 | NB | NB | NB | NB |
| MAb16665 | 330 | −1 | NB | NB | NB | NB |
| MAb16666 | 346 | 0 | NB | NB | NB | NB |
| MAb16669 | 166 | −1 | NB | NB | NB | NB |
| MAb16673 | 173 | 1 | NB | NB | NB | NB |
| MAb16676 | 187 | 0 | NB | NB | NB | NB |
| MAb16680 | 283 | 0 | NB | NB | NB | NB |
| MAb16682 | 204 | 0 | NB | NB | NB | NB |
| MAb16683 | 255 | 0 | NB | NB | NB | NB |
| MAb16684 | 214 | −1 | NB | NB | NB | NB |
| MAb16687 | 179 | 1 | NB | NB | NB | NB |
| MAb16692 | 191 | 0 | NB | NB | NB | NB |
| MAb16693 | 174 | 0 | NB | NB | NB | NB |
| MAb16695 | 185 | −1 | NB | NB | NB | NB |
| MAb16702 | 211 | 0 | NB | NB | NB | NB |
| IgG4 Isotype | 253 | 0 | NB | NB | NB | NB |
| Control | | | | | | |

NB indicates that no binding was observed under the current experimental conditions.

None of the anti-GP130 monoclonal antibodies of the invention bound to mGP130-MMH at 25° C. or at 37° C. as shown in Tables 9 and 10.

TABLE 11
Binding kinetics parameters of rGP130-MMH binding
to GP130 monoclonal antibodies at 25° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 142 ± 0.12 | 0 | NB | NB | NB | NB |
| MAb16618 | 180 ± 0.54 | 6 | NB | NB | NB | NB |
| MAb16622 | 196 ± 0.63 | 0 | NB | NB | NB | NB |
| MAb16623 | 240 ± 0.25 | 0 | NB | NB | NB | NB |
| MAb16636 | 194 ± 0.4 | 1 | NB | NB | NB | NB |
| MAb16637 | 306 ± 2.86 | 0 | NB | NB | NB | NB |
| MAb16641 | 153 ± 0.22 | −2 | NB | NB | NB | NB |
| MAb16646 | 166 ± 0.18 | 17 | 3.84E+04 | 1.32E−02 | 3.44E−07 | 0.9 |
| MAb16656 | 188 ± 1.11 | −1 | NB | NB | NB | NB |
| MAb16659 | 210 ± 0.3 | 2 | NB | NB | NB | NB |
| MAb16662 | 213 ± 0.1 | −1 | NB | NB | NB | NB |
| MAb16664 | 215 ± 0.95 | 0 | NB | NB | NB | NB |
| MAb16665 | 291 ± 0.16 | 0 | NB | NB | NB | NB |
| MAb16666 | 316 ± 1.11 | 1 | NB | NB | NB | NB |
| MAb16669 | 151 ± 0.14 | −2 | NB | NB | NB | NB |
| MAb16673 | 157 ± 0.13 | 0 | NB | NB | NB | NB |
| MAb16676 | 174 ± 0.16 | 0 | NB | NB | NB | NB |
| MAb16680 | 267 ± 1 | 2 | NB | NB | NB | NB |
| MAb16682 | 184 ± 0.2 | 0 | NB | NB | NB | NB |
| MAb16683 | 233 ± 0.76 | 0 | NB | NB | NB | NB |
| MAb16684 | 202 ± 0.47 | 1 | NB | NB | NB | NB |
| MAb16687 | 170 ± 0.18 | 0 | NB | NB | NB | NB |
| MAb16692 | 180 ± 0.67 | 8 | 1.78E+05 | 8.19E−02 | 4.60E−07 | 0.14 |
| MAb16693 | 158 ± 0.01 | −1 | NB | NB | NB | NB |
| MAb16695 | 183 ± 0.21 | 0 | NB | NB | NB | NB |
| MAb16702 | 206 ± 0.36 | 1 | NB | NB | NB | NB |
| IgG4 Isotype | 226 ± 0.27 | 0 | NB | NB | NB | NB |
| Control | | | | | | |

NB indicates that no binding was observed under the current experimental conditions.

TABLE 12
Binding kinetics parameters of rGP130-MMH binding
to GP130 monoclonal antibodies at 37° C.

| | | 100 nM | | | | |
|---|---|---|---|---|---|---|
| | mAb | Ag | | | | |
| | Capture | Bound | ka | kd | KD | t½ |
| mAb Captured | Level (RU) | (RU) | (1/Ms) | (1/s) | (M) | (min) |
| MAb16614 | 167 ± 0.23 | 0 | NB | NB | NB | NB |
| MAb16618 | 207 ± 0.91 | 2 | NB | NB | NB | NB |
| MAb16622 | 227 ± 0.51 | 1 | NB | NB | NB | NB |
| MAb16623 | 270 ± 0.14 | 0 | NB | NB | NB | NB |
| MAb16636 | 219 ± 0.61 | 0 | NB | NB | NB | NB |
| MAb16637 | 337 ± 1.83 | 0 | NB | NB | NB | NB |
| MAb16641 | 177 ± 0.86 | −1 | NB | NB | NB | NB |
| MAb16646 | 199 ± 0.44 | 7 | 1.77E+05 | 7.12E−02 | 4.03E−07 | 0.16 |
| MAb16656 | 207 ± 0.08 | 0 | NB | NB | NB | NB |
| MAb16659 | 229 ± 0.21 | 1 | NB | NB | NB | NB |
| MAb16662 | 235 ± 0.57 | 1 | NB | NB | NB | NB |
| MAb16664 | 243 ± 0.44 | 0 | NB | NB | NB | NB |
| MAb16665 | 328 ± 1.24 | −1 | NB | NB | NB | NB |
| MAb16666 | 341 ± 2.48 | 1 | NB | NB | NB | NB |
| MAb16669 | 166 ± 0.15 | −2 | NB | NB | NB | NB |
| MAb16673 | 172 ± 0.18 | 2 | NB | NB | NB | NB |
| MAb16676 | 186 ± 0.58 | 0 | NB | NB | NB | NB |
| MAb16680 | 282 ± 0.79 | 1 | NB | NB | NB | NB |
| MAb16682 | 203 ± 0.54 | 1 | NB | NB | NB | NB |
| MAb16683 | 256 ± 0.27 | 0 | NB | NB | NB | NB |
| MAb16684 | 212 ± 1.24 | −2 | NB | NB | NB | NB |
| MAb16687 | 179 ± 0.18 | 1 | NB | NB | NB | NB |
| MAb16692 | 191 ± 0.49 | 7 | 2.39E+05 | 2.27E−01 | 9.50E−07 | 0.05 |
| MAb16693 | 174 ± 0.66 | 1 | NB | NB | NB | NB |
| MAb16695 | 184 ± 0.36 | 0 | NB | NB | NB | NB |
| MAb16702 | 210 ± 0.16 | 1 | NB | NB | NB | NB |
| IgG4 Isotype | 252 ± 0.45 | 1 | NB | NB | NB | NB |
| Control | | | | | | |

NB indicates that no binding was observed under the current experimental conditions.

As shown in Tables 11 and 12, 2 out of 26 anti-GP130 monoclonal antibodies of the invention bound to rGP130-MMH. At 25° C., anti-GP130 monoclonal antibodies bound to rGP130-MMH with K_{D} values ranging from 344 nM to 460 nM, as shown in Table 11. At 37° C., anti-GP130 monoclonal antibodies bound to rGP130-MMH with K_{D} values ranging from 403 nM to 950 as shown in Table 12.

### Example 4: Anti-GP130 Antibody Cell Binding by FACS Analysis

In order to assess cell binding by anti-GP130 antibodies of the invention two cell lines were generated. One cell line generated was HEK293 cells stably over-expressing full length (FL) human gp130 (amino acids 1-918 of accession #P40189 with leucine at position 2 changed to valine, a natural variant) along with a luciferase reporter (Stat3-luciferase, Stat3-luc, SA Bioscience, #CLS-6028L). The cells were sorted twice using flow cytometry for high expression of gp130. It is known hereafter as HEK293/Stat3-luc/gp130-2 X Sort. IMR-32 cells (human Neuroblastoma, ATCC), were also evaluated for cell binding as these cells express gp130 endogenously and were used for bioassays. The cells used for binding were generated to stably express a luciferase reporter (Stat3-luciferase, Stat3-luc, SA Bioscience, #CLS-6028L), and are referred to hereafter as IMR-32/STAT3-Luc cells.

For the FACS analysis, 10 nM of the antibodies were used to stain 0.5×10⁶ cells/well of each cell type at 4° C. in PBS (without calcium and magnesium) containing 2% FBS for 30 minutes. IMR-32/STAT3-luc cells were incubated with 1 mg/mL mouse IgG for 30 minutes at 4° C. to block Fc receptors prior to adding the antibodies. To test whether the anti-gp130 antibody binding is specific for gp130 on the IMR-32 cells, antibodies were added to IMR-32/Stat3-luc cells with or without being pre-bound to 1000 nM of recombinant protein of the ecto-domain of human gp130 fused to myc-myc-his tag (hgp130.mmh) for 30 minutes at 25° C. After incubation with primary antibodies, the cells were stained with 8 μg/mL of Alexa Fluor®-647 conjugated secondary antibody (Jackson ImmunoResearch Laboratories Inc., anti-human #109-607-003) for 30 minutes. Cells were fixed using BD CytoFix™ (Becton Dickinson, #554655) and analyzed on an IQue® (Intellicyt®) Flow Cytometer. Unstained and secondary antibody alone controls were also tested for all cell lines. The results were analyzed using ForeCyt® (IntelliCyt®) software to determine the geometric means of fluorescence for viable cells.

As shown in Table 13, twenty-six anti-gp130 antibodies of the invention tested at 10 nM demonstrated binding to HEK293/Stat3-luc/gp130-2 X Sort cells with binding ratios ranging from 29- to 159-fold. The anti-gp130 antibodies demonstrated binding to the HEK293 parental cells with binding ratios 4- to 45-fold. Binding ratios to the IMR-32/Stat3-luc cells ranged from 5- to 23-fold without hgp130.mmh and from 4- to 9-fold with hGP130.mmh. The isotype control antibodies and secondary antibodies alone samples demonstrated binding ratios ranging from 1- to 3-fold for HEK293 cell lines, and 1- to 8-fold for IMR-32/Stat3-luc cells.

TABLE 13
Binding of 10 nM anti-gp130 antibodies to HEK293/Stat3-luc/gp130-2X
Sort and IMR-32/Stat3-luc cells.

| | MFI - Normalized to Unstained Control | | | |
|---|---|---|---|---|
| | | | IMR-32/Stat3-luc | |
| | 293 | 293/Stat3-luc/ | No | 1 uM |
| Antibody | Parental | GP130 2X sort | hGP130.mmh | hGP130.mmh |
| MAb16614 | 13 | 94 | 7 | 6 |
| MAb16618 | 13 | 105 | 7 | 8 |
| MAb16622 | 9 | 91 | 5 | 7 |
| MAb16623 | 14 | 96 | 8 | 7 |
| MAb16636 | 20 | 92 | 8 | 7 |
| MAb16637 | 22 | 121 | 9 | 7 |
| MAb16641 | 13 | 81 | 8 | 7 |
| MAb16646 | 6 | 29 | 5 | 6 |
| MAb16656 | 20 | 132 | 12 | 7 |
| MAb16659 | 14 | 81 | 8 | 8 |
| MAb16662 | 7 | 49 | 5 | 6 |
| MAb16664 | 8 | 45 | 7 | 8 |
| MAb16665 | 4 | 55 | 5 | 6 |
| MAb16666 | 45 | 159 | 23 | 8 |
| MAb16669 | 9 | 53 | 6 | 5 |
| MAb16673 | 15 | 107 | 10 | 7 |
| MAb16676 | 13 | 74 | 9 | 8 |
| MAb16680 | 21 | 99 | 11 | 9 |
| MAb16682 | 5 | 80 | 6 | 8 |
| MAb16683 | 13 | 75 | 9 | 7 |
| MAb16684 | 7 | 60 | 5 | 4 |
| MAb16687 | 12 | 71 | 9 | 7 |
| MAb16692 | 23 | 103 | 12 | 6 |
| MAb16693 | 4 | 51 | 7 | 7 |
| MAb16695 | 13 | 64 | 10 | 9 |
| MAb16702 | 25 | 111 | 11 | 6 |
| hIg4 Isotype | 3 | 2 | 6 | 7 |
| Control | | | | |
| a-h 2″ alone | 2 | 2 | 4 | 8 |
| Unstained | 1 | 1 | 1 | 1 |

### Example 5: Octet Cross-Competition Between Different Anti-GP130 Monoclonal Antibodies

Binding competition between a panel of anti-GP130 monoclonal antibodies was determined using a real time, label-free bio-layer interferometry assay on the Octet HTX biosensor platform (Pall ForteBio Corp.). The entire experiment was performed at 25° C. in 10 mM HEPES, 150 mM NaCl, 3 mM EDTA, 0.05% v/v Surfactant Tween-20, and 1 mg/mL BSA, pH7.4 (HBS-EBT) buffer with the plate shaking at the speed of 1000 rpm. To assess whether 2 antibodies compete with one another for binding to their respective epitopes on the recombinant human GP130 (hGP130-mmH; expressed with a C-terminal myc-myc-hexahistidine tag SEQ ID:188), about 0.4-0.5 nm of hGP130-mmH was first captured onto anti-Penta-His antibody coated Octet biosensor tips (Fortebio Inc, #18-5122) by submerging the biosensor tips for 4 minutes in wells containing 40-501.1 g/mL solution of hGP130-MMH. The antigen captured biosensor tips were then saturated with the first anti-GP130 monoclonal antibody (mAb-1) by dipping into wells containing 50 μg/mL solution of mAb-1 for 4 minutes. The biosensor tips were then dipped into wells containing 50 μg/mL solution of the second anti-GP130 monoclonal antibody (mAb-2) for 3 minutes. The biosensor tips were washed in HBS-ETB buffer between every step of the experiment. The real-time binding response was monitored during the entire course of the experiment and the binding response at the end of every step was recorded. The response of mAb-2 binding to hGP130-MMH pre-complexed with mAb-1 was compared and competitive/non-competitive behavior of different anti-GP130 monoclonal antibodies was determined as shown in Table 14.

TABLE 14
Cross-competition between anti-GP130 monoclonal antibodies

| mAb-1 | mAb-2 that competes with mAb-1 |
|---|---|
| MAb16659 | MAb16676 |
| | MAb16695 |
| | MAb16664 |
| | MAb16622 |
| | MAb16637 |
| MAb16676 | MAb16659 |
| | MAb16695 |
| | MAb16664 |
| | MAb16622 |
| | MAb16637 |
| MAb16695 | MAb16659 |
| | MAb16676 |
| | MAb16664 |
| | MAb16622 |
| | MAb16637 |
| MAb16664 | MAb16659 |
| | MAb16676 |
| | MAb16695 |
| | MAb16622 |
| | MAb16637 |
| MAb16622 | MAb16659 |
| | MAb16676 |
| | MAb16695 |
| | MAb16664 |
| | MAb16637 |
| | MAb16665 |
| | MAb16687 |
| | MAb16656 |
| MAb16637 | MAb16659 |
| | MAb16676 |
| | MAb16695 |
| | MAb16664 |
| | MAb16622 |
| | MAb16665 |
| | MAb16687 |
| | MAb16656 |
| MAb16665 | MAb16622 |
| | MAb16637 |
| | MAb16687 |
| | MAb16656 |
| MAb16687 | MAb16622 |
| | MAb16637 |
| | MAb16665 |
| | MAb16656 |
| MAb16656 | MAb16622 |
| | MAb16637 |
| | MAb16665 |
| | MAb16687 |
| MAb16641 | No mAb |
| MAb16666 | MAb16662 |
| MAb16662 | MAb16666 |
| MAb16618 | MAb16673 |
| | MAb16636 |
| | MAb16692 |
| MAb16673 | MAb16618 |
| | MAb16636 |
| | MAb16692 |
| MAb16636 | MAb16618 |
| | MAb16673 |
| | MAb16692 |
| | MAb16614 |
| | MAb16682 |
| MAb16692 | MAb16618 |
| | MAb16673 |
| | MAb16636 |
| | MAb16614 |
| | MAb16682 |
| MAb16614 | MAb16636 |
| | MAb16692 |
| | MAb16682 |
| MAb16682 | MAb16636 |
| | MAb16692 |
| | MAb16614 |
| MAb16623 | MAb16684 |
| | MAb16702 |
| | MAb16693 |
| | MAb16669 |
| MAb16684 | MAb16623 |
| | MAb16702 |
| | MAb16693 |
| | MAb16669 |
| MAb16702 | MAb16623 |
| | MAb16684 |
| | MAb16693 |
| | MAb16669 |
| MAb16693 | MAb16623 |
| | MAb16684 |
| | MAb16702 |
| | MAb16669 |
| MAb16669 | MAb16623 |
| | MAb16684 |
| | MAb16702 |
| | MAb16693 |
| MAb16646 | MAb16680 |
| MAb16680 | MAb16646 |
| MAb16683 | No mAb |

### Example 6: Monoclonal Antibodies Binding to GP130 Domain Proteins in Luminex-GP130 Delta D1-mmH and GP130 Delta D1-3-mmH CHO Supernatant

To identify the binding region of human GP130 with which anti-GP130 antibodies of the invention interact, a Luminex FLEXMAP (FM3DD, LuminexCorp) flow cytometry based analysis was utilized to characterize the interaction of recombinant human GP130 protein domains. For the assay, approximately 3 million carboxylated Microplex^{R} microspheres (Luminex, Cat #LC1000A), were washed, vortexed and sonicated in 0.1 M NaPO₄, pH 6.2 (activation buffer) and then centrifuged to remove the supernatant. The microspheres were re-suspended in 120 μL of activation buffer and the carboxylate groups (—COOH) were activated by addition of 15 μL of 50 mg/mL of N-hydroxysuccinimide (NHS, Thermo Scientific, Cat #24500) followed by addition of 15 μL of 50 mg/mL of 1-ethyl-3-[3-dimethylaminopropyl]carbodiimide (EDC, ThermoScientific, Cat #22980) at 25° C. After 10 minutes, the pH of the reaction was reduced to 5.0 with the addition of 600 μL of 50 mM MES, pH 5 (coupling buffer), and the microspheres were vortexed, and centrifuged to remove supernatant. The activated beads were immediately mixed with 500 μL of 20 μg/mL monoclonal anti-myc antibodies with mouse IgG, in coupling buffer and incubated for two hours at 25° C. The coupling reaction was quenched by addition of 50 μL of 1M TRIS-HCl, pH 8.0 and the microspheres were rapidly vortexed, centrifuged, and washed four times with 1 mL of Dulbecco's Phosphate Buffered Saline (DPBS, pH 7.2, ThermoScientific Cat #14190136), to remove uncoupled proteins and other reaction components.

The transiently expressed GP130 proteins, including human GP130 delta D1 expressed with a C-terminal myc-myc hexahistidine tag (human GP130 delta D1-MMH, SEQ ID NO:192) and human GP130 delta D1-D3 expressed with a C-terminal myc-myc hexahistidine tag (human GP130 delta D1-D3-MMH, SEQ ID NO:193), were suspended in serum free CHO—S-SFM II Medium (Thermo Fisher, Cat #31033020) and were then clarified by centrifugation. Purified GP130 full length extracellular domain expressed with a C-terminal myc-myc hexahistidine tag (human GP130-MMH, SEQ ID NO:191) was prepared at 10 ug/mL in PBS. Aliquots of microspheres with immobilized anti-myc monoclonal antibodies, prepared as described above, were added individually to 1 mL of the each of these protein supernatants and to 500 uL of purified GP130 protein. The microspheres were gently mixed, incubated for two hours at 25° C., washed twice with 1 mL of DBPS, centrifuged to remove the supernatant and finally resuspended in 1 mL of DPBS buffer. Forty eight μL of anti-myc IgG coupled microspheres from individual reactions with full length human GP130 and with each of the human GP130 domain proteins were withdrawn and mixed together in 3.6 mL of PBS+20 mg/mL BSA+0.05% sodium azide (blocking buffer).

From this mixed pool, 75 μL of microspheres were plated per well on a 96 well filter plate (Millipore, Cat. No: MSBVN1250) and mixed with 25 μL of individual anti-human GP130 monoclonal antibodies (0.5 or 5 μg/mL), incubated for two hours at 25° C. and then washed twice with 200 μL of DPBS with 0.05% Tween 20 (washing buffer). To detect and quantify the amounts of bound anti-GP130 antibody levels to individual microspheres, either 100 μL of 2.5 μg/mL R-Phycoerythrin conjugated goat F(ab′)2 anti-human kappa (Southern Biotech, Cat #2063-09) in blocking buffer, was added and incubated for 30 minutes at 25° C. After 30 minutes, the samples were washed twice with 200 μL of washing buffer and resuspended in 150 μL of wash buffer. The Median Fluorescence intensity (MFI) of the microspheres was measured in a Luminex Analyzer.

TABLE 15
Luminex MFI signal of anti-GP130 antibodies binding to myc tag
captured full-length extracellular domain of human GP130, isolated
human GP130 delta D1 and delta D1-D3 domains.

| | GP130 | GP130 | GP130 | |
|---|---|---|---|---|
| | Delta D1 | Delta D1-D3 | Full length | |
| | extracellular | extracellular | extracellular | Interacting |
| Antibody | domain | domain | domain | Domain(s) |
| mAb16614 | 2899 | 56 | 18690 | D2-D3 |
| mAb16618 | 2634 | 19 | 18986 | D2-D3 |
| mAb16622 | 391 | 20 | 18462 | D2-D3 |
| mAb16623 | 12565 | 16012 | 17617 | FNIII |
| mAb16636 | 4261 | 1067 | 17617 | D2-D3 |
| mAb16637 | 342 | 861 | 16899 | D1 |
| mAb16641 | 51 | 27 | 17298 | D1 |
| mAb16646 | 11329 | 18005 | 13957 | FNIII |
| mAb16656 | 48 | 17 | 15911 | D1 |
| mAb16659 | 49 | 22 | 17092 | D1 |
| mAb16662 | 3050 | 16 | 13926 | D2-D3 |
| mAb16664 | 442 | 419 | 19306 | D1 |
| mAb16665 | 119 | 116 | 16332 | D1 |
| mAb16666 | 7280 | 319 | 16676 | D2-D3 |
| mAb16669 | 12643 | 15063 | 17640 | FNIII |
| mAb16673 | 2344 | 33 | 15065 | D2-D3 |
| mAb16676 | 364 | 686 | 18074 | D1 |
| mAb16680 | 10911 | 18713 | 14422 | FNIII |
| mAb16682 | 1380 | 13 | 13825 | D2-D3 |
| mAb16683 | 12026 | 20857 | 15220 | FNIII |
| mAb16684 | 9126 | 11992 | 14398 | FNIII |
| mAb16687 | 37 | 18 | 15485 | D1 |
| mAb16692 | 3893 | 17 | 16202 | D2-D3 |
| mAb16693 | 9449 | 12009 | 12943 | FNIII |
| mAb16695 | 30 | 7 | 14533 | D1 |
| mAb16702 | 11721 | 12500 | 14951 | FNIII |

The results of the Luminex based analysis are tabulated in Table 15. Luminex MFI signal intensities indicate that the twenty six anti-GP130 antibodies of the invention bound to the human GP130 full length extracellular domain.

Anti-GP130 antibodies mAb16637, mAb16641, mAb16656, mAb16659, mAb16664, mAb16665, mAb16676, mAb16687 and MAb16695 lost binding to both deletion proteins, suggesting binding epitopes within the D1 domain of human GP130.

Anti-GP130 antibodies mAb16614, mAb16618, mAb16622, mAb16636, mAb16662, mAb16666, mAb16673, mAb16682, mAb16692 lost binding to GP130 delta D1-D3 while retaining binding to GP130 delta D1, indicating their binding epitope is within domains D2-D3 of human GP130.

Anti-GP130 antibodies mAb16623, mAb16646, mAb16669, mAb16680, mAb16683, mAb16684, mAb16693, mAb16702 bound to GP130 delta D1 and GP130 delta D1-D3, indicating their binding domain is within FNIII of human GP130.

### Example 7: Functional Cell-Based Assay with in IMR-32/Stat3-Luc Cells, without Ligands or with hOncostain M, hLIF, or hCNTF

In order to assess transcriptional activation or inhibition of anti-GP130 antibodies, IMR-32 cells (human Neuroblastoma ATCC) were generated to stably express a luciferase reporter (STAT3-Luc; SABiosciences, #CLS-6028L). The resulting cell line is referred to hereafter as IMR-32/STAT3-Luc (see Example 4 herein).

For the bioassay, IMR-32/STAT3-Luc cells were plated at 15,000 cells/well in a 96-well plate in assay buffer (0.1% FBS in Optimem with pen/strep) and incubated overnight at 37° C. in 5% CO₂. The following day anti-gp130 antibodies or an isotype control were serially diluted from 100 nM to 24.4 pM in assay buffer (plus a sample containing buffer alone without test molecule), added to the cells and incubated at 25° C. for 30 minutes. After 30 minutes, either 100 pM human Oncostatin M (hOSM, R&D System 293-OM), 20 pM human Leukemia Inhibitory Factor (hLIF, R&D Systems 7734-LF), 20 pM human Ciliary Neurotrophic Factor (hCNTF, R&D Systems 257-NT), or assay buffer was added to cells. hOSM, hLIF, and hCNTF were serially diluted from 10 nM to 0.17 pM in assay buffer (plus a sample containing buffer alone without test molecule) and added to cells not treated with antibodies. After 5 hours at 37° C. in 5% CO₂, luciferase activity was measured with OneGlo™ reagent (Promega, #E6031) and Victor™ X multilabel plate reader (Perkin Elmer). The results were analyzed using nonlinear regression (4-parameter logistics) with Prism 5 software (GraphPad) to obtain EC₅₀ and IC₅₀ values. Activation of antibodies was calculated with the maximum range of RLU achieved by the antibody over the maximum range of RLU achieved by hOSM. The percentage of inhibition was calculated with the RLU values by using the following equation:

$$\% Inhibition = 100 \times \frac{{RLU}_{Baseline} - {RLU}_{Inhibition}}{{RLU}_{Baseline} - {RLU}_{Background}}$$

In this equation “RLUBaseline” is the luminescence value from the cells treated with a constant amount of ligand (hOSM, hLIF, or hCNTF) without antibodies. “RLUInhibition” is the luminescence value with 100 nM of a particular antibody with a particular concentration of ligand, and “RLUBackground” is the luminescence value from cells without any ligands or antibodies.

As shown in Table 16, twenty-six anti-human gp130 antibodies of the invention were tested for their ability to either activate or inhibit activation of IMR-32/Stat3-luc cells. As shown in Table 19, in the absence of any added ligands none of the antibodies of the invention tested showed any activation of IMR-32/Stat3-luc cells. One of the 26 antibodies of the invention, MAb16692, showed complete inhibition of all three ligands tested with 1050 values of 48 pM, 140 pM, and 230 pM for hOSM, hLIF, and hCNTF, respectively. An additional ten antibodies of the invention tested showed some inhibition of at least one of the ligands with the % inhibition ranging from 17% to 95%, with IC₅₀ values for the inhibiting antibodies ranging from >100 nM to 88 pM. Fifteen antibodies of the invention did not show inhibition of any of ligands tested. An isotype control antibody did not demonstrate any measureable activation or inhibition of IMR-32/Stat3-luc cells. The ligands activated IMR-32/STAT3-luc cells with EC₅₀ values of 54 pM for hOSM, 23 pM for hLIF, and 4 pM for hCNTF.

TABLE 16
Activation and inhibition of anti-gp130 antibodies in the absence or presence of GP130 liqands in IMR-32/Stat3-luc cells.

| | OSM (100 pM) | | LIF (20 pM) | | CNTF (20 pM) | | No Ligand |
|---|---|---|---|---|---|---|---|
| mAb PID | IC50 [M] | % inhibition | IC50 [M] | % inhibition | IC50 [M] | % inhibition | EC50 [M] |
| mAb16614 | No Inhibition | No Inhibition | No Inhibition | No Inhibition | No Inhibition | No Inhibition | No Activation |
| mAb16618 | >5.0E−08 | 52 | >1.0E−07 | 40 | >1.0E−07 | 33 | No Activation |
| mAb16622 | No Inhibition | No Inhibition | No Inhibition | No Inhibition | No Inhibition | No Inhibition | No Activation |
| mAb16623 | No Inhibition | No Inhibition | No Inhibition | No Inhibition | >1.0E−07 | 22 | No Activation |
| mAb16636 | >1.0E−08 | 82 | >1.0E−08 | 76 | >1.0E−08 | 62 | No Activation |
| mAb16637 | No Inhibition | No Inhibition | No Inhibition | No Inhibition | No Inhibition | No Inhibition | No Activation |
| mAb16641 | 8.8E−11 | 21 | No Inhibition | No Inhibition | No Inhibition | No Inhibition | No Activation |
| mAb16646 | >1.0E−07 | 47 | >1.0E−07 | 26 | >1.0E−07 | 30 | No Activation |
| mAb16656 | No Inhibition | No Inhibition | No Inhibition | No Inhibition | No Inhibition | No Inhibition | No Activation |
| mAb16659 | No Inhibition | No Inhibition | No Inhibition | No Inhibition | No Inhibition | No Inhibition | No Activation |
| mAb16662 | No Inhibition | No Inhibition | No Inhibition | No Inhibition | No Inhibition | No Inhibition | No Activation |
| mAb16664 | No Inhibition | No Inhibition | No Inhibition | No Inhibition | No Inhibition | No Inhibition | No Activation |
| mAb16665 | No Inhibition | No Inhibition | No Inhibition | No Inhibition | No Inhibition | No Inhibition | No Activation |
| mAb16666 | No Inhibition | No Inhibition | No Inhibition | No Inhibition | 7.1E−10 | 95 | No Activation |
| mAb16669 | No Inhibition | No Inhibition | No Inhibition | No Inhibition | No Inhibition | No Inhibition | No Activation |
| mAb16673 | >5.0E−08 | 62 | 9.0E−09 | 52 | >1.0E−07 | 46 | No Activation |
| mAb16676 | No Inhibition | No Inhibition | No Inhibition | No Inhibition | No Inhibition | No Inhibition | No Activation |
| mAb16680 | 1.8E−09 | 71 | 3.4E−09 | 80 | 9.6E−10 | 35 | No Activation |
| mAb16682 | >1E−07 | 23 | No Inhibition | No Inhibition | No Inhibition | No Inhibition | No Activation |
| mAb16683 | No Inhibition | No Inhibition | No Inhibition | No Inhibition | No Inhibition | No Inhibition | No Activation |
| mAb16684 | No Inhibition | No Inhibition | No Inhibition | No Inhibition | No Inhibition | No Inhibition | No Activation |
| mAb16687 | No Inhibition | No Inhibition | No Inhibition | No Inhibition | No Inhibition | No Inhibition | No Activation |
| mAb16692 | 4.8E−11 | 101 | 1.4E−10 | 101 | 2.3E−10 | 100 | No Activation |
| mAb16693 | No Inhibition | No Inhibition | No Inhibition | No Inhibition | No Inhibition | No Inhibition | No Activation |
| mAb16695 | No Inhibition | No Inhibition | No Inhibition | No Inhibition | No Inhibition | No Inhibition | No Activation |
| mAb16702 | No Inhibition | No Inhibition | No Inhibition | No Inhibition | >1E−07 | 17 | No Activation |
| Isotype Control mAb | No Inhibition | No Inhibition | No Inhibition | No Inhibition | No Inhibition | No Inhibition | No Activation |

### Example 8: GP130 Purified Antibodies Blocking ELISA

GP130 (glycoprotein 130) is a type I cytokine receptor transmembrane protein which forms a high-affinity ternary complex with the ligand ciliary neurotropic factor (CNTF) when it is associated with CNTFRα (ciliary neurotropic factor receptor alpha subunit). The ability of anti-gp130 antibodies to block GP130 protein binding to plate bound CNTFRα/CNTF complex was measured using a competition sandwich ELISA. In this assay, various concentrations of anti-gp130 antibody were pre-mixed with a constant amount of dimeric GP130 protein and the reduction of the gp130 binding due to the presence of the antibody to the plate immobilized CNTFRα/CNTF complex was monitored.

The dimeric gp130 protein used in the experiments was comprised of a portion of the human gp130 extracellular domain (amino acids E23-E619 of accession number NP 002175.2) expressed with the Fc portion of the mouse IgG2a protein at the c-terminus (hGP130-mFc; SEQ ID:190, mw 94,210 daltons). The CNTFRαprotein was purchased from R&D Systems (amino acids Q23-P346 of accession #6992, mw 36,000 daltons). The CNTF protein was purchased from R&D Systems (amino acids A2- M200 of accession #6441.1, mw 22,800 daltons). Isotype antibody control, anti-Fel d 1, and human IgG4^{P} antibody were included as controls for IgG background detection.

The experiment was carried out using the following procedure. Human CNTFRα was coated at a concentration of 2 mg/mL in HBSS on a 96-well microtiter plate overnight at 4° C. Nonspecific binding sites were subsequently blocked using a 1% (w/v) solution of BSA in HBSS. Human CNTF at a concentration of 1 μg/ml in HBSS was added to the plate bound CNTFRα for 1 hour at room temperature. In separate dilution plates, a constant amount of 2.5 nM of human GP130-mFc protein was titrated with antibodies ranging from 3.4 pM to 200 nM in serial dilution and with no antibody present. These solutions were incubated for 1 hour at room temperature (RT) and subsequently transferred to the microtiter plates with CNTFRα/CNTF complex without washing. The plates were incubated for 2 hours at RT, washed with PBST buffer, and plate-bound hGP130-mFc was detected with an anti-mFc polyclonal antibody conjugated with horseradish peroxidase (HRP) (Jackson ImmunoResearch Inc). Samples were developed with a TMB solution (BD Biosciences, substrate A and B mixed at 1:1 ratio as per manufacturer's instructions) to produce a colorimetric reaction and then neutralized with 1M sulfuric acid before measuring absorbance at 450 nm on a Victor X5 plate reader.

Data analysis was performed using a sigmoidal dose-response model within Prism™ software (GraphPad). The calculated 1050 value, defined as the concentration of antibody required to reduce 50% of GP130 binding to CNTFRα/CNTF complex, was used as an indicator of blocking potency. Percent blockade at maximum concentration tested was calculated as an indicator of the ability of the antibodies to block binding of GP130 to CNTFRα/CNTF on the plate as determined from the dose curve. The ratio of the reduction in signal observed in the presence of the highest tested concentration of 200 nM antibody, relative to the difference between the signal with 2.5 nM GP130 with no antibody (0% blocking) and the background signal from HRP-conjugated secondary antibody alone (100% blocking), was subtracted from 100% blocking.

The results of the blocking ELISA are shown in Table 17 with blocking percentages in the presence of 200 nM antibody reported for all antibodies. IC 50 values are reported only for antibodies blocking >30% of GP130 binding to CNTFRα/CNTF. Nineteen of twenty-six a-GP130 antibodies block <30% GP130 protein binding to plate-coated CNTFRα/CNTF. Negative numbers indicate an increase of GP130 binding detected in the presence of antibody. Seven antibodies blocked GP130 protein binding to CNTFRα/CNTF >30% and IC 50 values ranged from below the lower limit of quantitation for the assay of 1.25 nM to 6.5 nM, with four of them blocking 90% or more of the signal at the highest antibody concentration tested. The irrelevant blocking control antibody showed blocking of 4.5% at concentrations up to 200 nM.

TABLE 17
GP130 Purified Antibodies Blocking ELISA

| | | | Potency of Antibody |
|---|---|---|---|
| | | | Blocking 2.5 nM |
| | | | hGP130-mFc binding |
| | | Max Blocking | to plate-bound |
| Antibody | | of Antibody at | CNTFR/CNTF |
| Identifier | Lot # | 200 nM (%) | complex (M) |
| mAb16614 | MAb16614-L1 | −14.75 | — |
| mAb16618 | MAb16618-L1 | 86.65 | 2.374E−09 |
| mAb16622 | MAb16622-L1 | −84.14 | — |
| mAb16623 | MAb16623-L1 | −49.19 | — |
| mAb16636 | MAb16636-L1 | 94.76 | 1.104E−09 |
| mAb16637 | MAb16637-L1 | −38.86 | — |
| mAb16641 | MAb16641-L1 | 14.09 | — |
| mAb16646 | MAb16646-L1 | 5.24 | — |
| mAb16656 | MAb16656-L1 | −24.71 | — |
| mAb16659 | MAb16659-L1 | −18.81 | — |
| mAb16662 | MAb16662-L1 | 81.93 | 6.506E−09 |
| mAb16664 | MAb16664-L1 | −46.53 | — |
| mAb16665 | MAb16665-L1 | −47.12 | — |
| mAb16666 | MAb16666-L1 | 103.39 | 6.437E−10* |
| mAb16669 | MAb16669-L1 | 47.12 | 1.232E−08 |
| mAb16673 | MAb16673-L1 | 103.76 | 2.368E−09 |
| mAb16676 | MAb16676-L1 | −15.41 | — |
| mAb16680 | MAb16680-L1 | 11.95 | — |
| mAb16682 | MAb16682-L1 | −3.39 | — |
| mAb16683 | MAb16683-L1 | −22.79 | — |
| mAb16684 | MAb16684-L1 | 12.91 | — |
| mAb16687 | MAb16687-L1 | −17.40 | — |
| mAb16692 | MAb16692-L1 | 101.77 | 7.671E−10* |
| mAb16693 | MAb16693-L1 | 4.42 | — |
| mAb16695 | MAb16695-L1 | 21.17 | — |
| mAb16702 | MAb16702-L1 | 29.13 | — |
| hIgG4 Isotype | 07-120309 | 4.50 | — |
| Control | | | |

In this Example, 100% blocking is equal to OD450nm value HRP-conjugated secondary antibody with no GP130.
0% blocking is OD450nm value with 2.5 nM hGP130-mFc with no antibody.
Negative Max Blocking % indicates an increase of GP130 binding detected in the presence of antibody.
— IC₅₀ values are not quantitative for antibodies blocking <30% at the highest concentration tested.
*Indicates IC₅₀ value below the lower limit of quantitation of 1.25E−09M for the assay.

### Example 9: LEPR x GP130 Focused Approach Bispecific Screening

This Example describes the generation of bispecific antibodies that bind to both LEPR and GP130 for the promotion of STAT3 signaling. Such antibodies are referred to herein as “LEPR x GP130 bispecific antibodies,” or “LEPR x GP130 bsAbs,” “anti-LEPR x anti-GP130 bispecific antibodies,” or the like. In this Example, several anti-GP130 binding arms were paired with four different anti-LEPR binding arms. The anti-LEPR antibodies used to construct the bispecific antibodies of this Example are the agonistic antibodies referred to as mAb16679, mAb18445, mAb18446 and mAb18449 (see US Patent Appl. Publ. No. 2017/0101477, the disclosure of which is incorporated by reference herein in its entirety). The amino acid and nucleic acid sequences of the variable domains and CDRs of the anti-LEPR antibodies used in this Example are summarized in Tables 18 and 19, respectively.

TABLE 18
anti-LEPR Amino Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb16679 | 2 | 4 | 6 | 8 | 10 | 12 | 14 | 16 |
| mAb18449 | 162 | 164 | 166 | 168 | 10 | 12 | 14 | 16 |
| mAb18445 | 170 | 172 | 174 | 176 | 10 | 12 | 14 | 16 |
| mAb18446 | 178 | 180 | 182 | 184 | 10 | 12 | 14 | 16 |

TABLE 19
anti-LEPR Nucleic Acid Sequence Identifiers

| Antibody | SEQ ID NOs: | | | | | | | |
|---|---|---|---|---|---|---|---|---|
| Designation | HCVR | HCDR1 | HCDR2 | HCDR3 | LCVR | LCDR1 | LCDR2 | LCDR3 |
| mAb16679 | 1 | 3 | 5 | 7 | 9 | 11 | 13 | 15 |
| mAb18449 | 161 | 163 | 165 | 167 | 9 | 11 | 13 | 15 |
| mAb18445 | 169 | 171 | 173 | 175 | 9 | 11 | 13 | 15 |
| mAb18446 | 177 | 179 | 181 | 183 | 9 | 11 | 13 | 15 |

Eighteen bispecific antibodies were generated through pairing of anti-LEPR binding arms from mAb16679 with binding arms from 18 different anti-GP130 antibodies. Six additional bispecific antibodies were created by pairing binding arms from anti-LEPR antibodies mAb18449, mAb18445 and mAb18446 with anti-GP130 binding arms. Standard methods were used to produce the bispecific antibodies described herein. All LEPR x GP130 bispecific antibodies shown in this example comprise the same (“common”) light chain (comprising the light chain variable region [LCVR] amino acid sequence of SEQ ID NO:10, and light chain CDR [LCDR1, LCDR2 and LCDR3] amino acid sequences of SEQ ID NOs: 12, 14 and 16). The components of the bispecific antibodies of this Example are summarized in Table 20.

TABLE 20
LEPR × GP130 Bispecific Antibody Components Summary.

| | SEQ ID NOs: (Amino Acid Sequences) | | | | | | | |
|---|---|---|---|---|---|---|---|---|
| Bispecific | LEPR Binding Arm Domain (D1) | | | | GP130 Binding Arm Domain (D2) | | | |
| Antibody | D1-HCVR | D1-HCDR1 | D1-HCDR2 | D1-HCDR3 | D2-HCVR | D2-HCDR1 | D2-HCDR2 | D2-HCDR3 |
| bsAb19139D | mAb16679 | | | | mAb 16614 | | | |
| | 2 | 4 | 6 | 8 | 18 | 20 | 22 | 24 |
| bsAb19140D | mAb 16679 | | | | mAb 16618 | | | |
| | 2 | 4 | 6 | 8 | 26 | 28 | 30 | 32 |
| bsAb19141D | mAb 16679 | | | | mAb 16622 | | | |
| | 2 | 4 | 6 | 8 | 34 | 36 | 38 | 40 |
| bsAb19142D | mAb 16679 | | | | mAb 16623 | | | |
| | 2 | 4 | 6 | 8 | 42 | 44 | 46 | 48 |
| bsAb19143D | mAb 16679 | | | | mAb 16636 | | | |
| | 2 | 4 | 6 | 8 | 50 | 52 | 54 | 56 |
| bsAb19144D | mAb 16679 | | | | mAb 16637 | | | |
| | 2 | 4 | 6 | 8 | 58 | 60 | 62 | 64 |
| bsAb19145D | mAb 16679 | | | | mAb 16656 | | | |
| | 2 | 4 | 6 | 8 | 66 | 68 | 70 | 72 |
| bsAb19146D | mAb 16679 | | | | mAb 16659 | | | |
| | 2 | 4 | 6 | 8 | 74 | 76 | 78 | 80 |
| bsAb19147D | mAb16679 | | | | mAb16662 | | | |
| | 2 | 4 | 6 | 8 | 82 | 84 | 86 | 88 |
| bsAb19148D | mAb16679 | | | | mAb16664 | | | |
| | 2 | 4 | 6 | 8 | 90 | 92 | 94 | 96 |
| bsAb19149D | mAb16679 | | | | mAb16665 | | | |
| | 2 | 4 | 6 | 8 | 98 | 100 | 102 | 104 |
| bsAb19150D | mAb16679 | | | | mAb16666 | | | |
| | 2 | 4 | 6 | 8 | 106 | 108 | 110 | 112 |
| bsAb19151D | mAb16679 | | | | mAb16669 | | | |
| | 2 | 4 | 6 | 8 | 114 | 116 | 118 | 120 |
| bsAb19152D | mAb16679 | | | | mAb16673 | | | |
| | 2 | 4 | 6 | 8 | 122 | 124 | 126 | 128 |
| bsAb19153D | mAb16679 | | | | mAb16676 | | | |
| | 2 | 4 | 6 | 8 | 130 | 132 | 134 | 136 |
| bsAb19154D | mAb16679 | | | | mAb16680 | | | |
| | 2 | 4 | 6 | 8 | 138 | 140 | 142 | 144 |
| bsAb19155D | mAb16679 | | | | mAb16682 | | | |
| | 2 | 4 | 6 | 8 | 146 | 148 | 150 | 152 |
| bsAb19156D | mAb16679 | | | | mAb16683 | | | |
| | 2 | 4 | 6 | 8 | 154 | 156 | 158 | 160 |
| bsAb19757D | mAb18449 | | | | mAb16622 | | | |
| | 162 | 164 | 166 | 168 | 34 | 36 | 38 | 40 |
| bsAb19758D | mAb18449 | | | | mAb16666 | | | |
| | 162 | 164 | 166 | 168 | 106 | 108 | 110 | 112 |
| bsAb21236D | mAb18445 | | | | mAb16683 | | | |
| | 170 | 172 | 174 | 176 | 154 | 156 | 158 | 160 |
| bsAb21237D | mAb18446 | | | | mAb16683 | | | |
| | 178 | 180 | 182 | 184 | 154 | 156 | 158 | 160 |
| bsAb27679D | mAb18445 | | | | mAb16683 | | | |
| | 170 | 172 | 174 | 176 | 154 | 156 | 158 | 160 |
| bsAb27680D | mAb18446 | | | | mAb16683 | | | |
| | 178 | 180 | 182 | 184 | 154 | 156 | 158 | 160 |

### Example 10: Biacore Binding Kinetics of Anti-LEPR x Anti-GP130 Bispecific Antibodies Binding to Different GP130 Reagents Measured at 25° C. and 37° C.

Equilibrium dissociation constants (K_{D} values) for LEPR and GP130 binding to purified anti-LEPR/GP130 bispecific antibodies were determined using a real-time surface plasmon resonance biosensor using a Biacore 4000 instrument. All binding studies were performed in 10 mM HEPES, 150 mM NaCl, 3 mM EDTA, and 0.05% v/v Surfactant Tween-20, pH 7.4 (HBS-ET) running buffer at 25° C. and 37° C. The Biacore sensor surface was first derivatized by amine coupling with a monoclonal mouse anti-human Fc antibody (GE, #BR-1008-39) to capture anti-LEPR/GP130 bispecific antibodies. Binding studies were performed on following reagents: human LEPR extracellular domain expressed with a C-terminal myc-myc-hexahistidine tag (hLEPR-MMH; SEQ ID NO:187), Macaca Fascicularis LEPR extracellular domain expressed with a C-terminal myc-myc-hexahistidine tag (mfLEPR-MMH; SEQ ID NO: 188), human GP130 extracellular domain expressed with a C-terminal myc-myc-hexahistidine tag (hGP130-MMH; SEQ ID NO:191), and Macaca Fascicularis GP130 extracellular domain expressed with a C-terminal myc-myc-hexahistidine tag (mfGP130-MMH; SEQ ID NO:194). Different concentrations of LEPR or GP130 reagents were first prepared in HBS-ET running buffer (100 nM-3.7 nM; 3-fold serial dilution) and were injected over anti-human Fc captured anti-LEPR/GP130 bispecific antibody surface for 4 minutes at a flow rate of 30 μL/minute, while the dissociation of bispecific antibody bound LEPR or GP130 reagent was monitored for 10 minutes in HBS-ET running buffer. Kinetic association (kₐ) and dissociation (k_{d}) rate constants were determined by fitting the real-time binding sensorgrams to a 1:1 binding model with mass transport limitation using Scrubber 2.0c curve-fitting software. Binding dissociation equilibrium constants (K_{D}) and dissociative half-lives (t ½) were calculated from the kinetic rate constants as:

$${K}_{D} ( M ) = \frac{kd}{ka} , and t 1 / 2 ( min ) = \frac{ln ( 2 )}{60 * kd} .$$

Binding kinetics parameters for hLEPR-MMH, mfLEPR-MMH, hGP130-MMH or mfGP130-MMH binding to different anti-LEPR/GP130 bispecific antibodies of the invention at 25° C. and 37° C. are shown in Tables 21 through 28.

TABLE 21
Binding kinetics parameters of hLEPR-MMH binding
to anti-LEPR/GP130 bispecific antibodies at 25° C.

| | | 100 nM | | | | |
|---|---|---|---|---|---|---|
| | mAb | hLEPR-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 418 ± 0.5 | 41 | 2.41E+04 | 1.71E−04 | 7.09E−09 | 68 |
| bsAb19140D | 444 ± 0.7 | 47 | 2.45E+04 | 1.80E−04 | 7.36E−09 | 64 |
| bsAb19141D | 458 ± 2.5 | 45 | 2.54E+04 | 1.51E−04 | 5.91E−09 | 77 |
| bsAb19142D | 452 ± 2.1 | 46 | 2.25E+04 | 1.75E−04 | 7.76E−09 | 66 |
| bsAb19143D | 446 ± 1.6 | 45 | 2.49E+04 | 1.82E−04 | 7.31E−09 | 64 |
| bsAb19144D | 456 ± 2.6 | 48 | 2.46E+04 | 1.59E−04 | 6.48E−09 | 73 |
| bsAb19145D | 437 ± 1.2 | 41 | 2.49E+04 | 1.65E−04 | 6.62E−09 | 70 |
| bsAb19146D | 461 ± 1 | 45 | 2.20E+04 | 1.79E−04 | 8.16E−09 | 64 |
| bsAb19147D | 439 ± 0.7 | 42 | 2.27E+04 | 1.62E−04 | 7.13E−09 | 71 |
| bsAb19148D | 452 ± 1.1 | 44 | 2.36E+04 | 1.72E−04 | 7.31E−09 | 67 |
| bsAb19149D | 446 ± 1.4 | 36 | 2.71E+04 | 1.94E−04 | 7.16E−09 | 60 |
| bsAb19150D | 451 ± 2.9 | 41 | 2.40E+04 | 1.76E−04 | 7.35E−09 | 66 |
| bsAb19151D | 419 ± 1.3 | 46 | 2.11E+04 | 1.58E−04 | 7.49E−09 | 73 |
| bsAb19152D | 455 ± 2 | 47 | 2.45E+04 | 1.65E−04 | 6.75E−09 | 70 |
| bsAb19153D | 441 ± 1.9 | 40 | 2.55E+04 | 1.65E−04 | 6.46E−09 | 70 |
| bsAb19154D | 438 ± 0.7 | 40 | 2.26E+04 | 1.80E−04 | 7.95E−09 | 64 |
| bsAb19155D | 431 ± 0.7 | 40 | 2.40E+04 | 1.53E−04 | 6.36E−09 | 76 |
| bsAb19156D | 439 ± 0.8 | 39 | 2.31E+04 | 1.64E−04 | 7.11E−09 | 70 |
| bsAb19757D | 432 ± 1.3 | 57 | 2.07E+04 | 1.62E−04 | 7.85E−09 | 71 |
| bsAb19758D | 428 ± 4.1 | 54 | 1.59E+04 | 1.63E−04 | 1.03E−08 | 71 |
| bsAb21236D | 78 ± 0.3 | 8 | 3.51E+04 | 3.72E−03 | 1.06E−07 | 3.1 |
| bsAb21237D | 206 ± 0.6 | 11 | 1.53E+04 | 1.21E−03 | 7.87E−08 | 10 |
| Isotype Control | 427 ± 0.8 | −2 | NB* | NB* | NB* | NB* |

*NB indicates that no binding was observed under the current experimental conditions.

TABLE 22
Binding kinetics parameters of hLEPR-MMH binding
to anti-LEPR/GP130 bispecific antibodies at 37° C.

| | | 100 nM | | | | |
|---|---|---|---|---|---|---|
| | mAb | hLEPR-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 527 ± 1.6 | 67 | 3.58E+04 | 7.62E−04 | 2.12E−08 | 15 |
| bsAb19140D | 559 ± 0.3 | 75 | 3.82E+04 | 8.18E−04 | 2.14E−08 | 14 |
| bsAb19141D | 582 ± 2.7 | 74 | 3.54E+04 | 5.89E−04 | 1.66E−08 | 20 |
| bsAb19142D | 551 ± 2.7 | 74 | 3.40E+04 | 6.33E−04 | 1.86E−08 | 18 |
| bsAb19143D | 558 ± 1.5 | 72 | 3.55E+04 | 7.63E−04 | 2.15E−08 | 15 |
| bsAb19144D | 568 ± 1.9 | 78 | 3.68E+04 | 5.97E−04 | 1.62E−08 | 19 |
| bsAb19145D | 558 ± 1.4 | 66 | 3.86E+04 | 8.05E−04 | 2.08E−08 | 14 |
| bsAb19146D | 576 ± 1 | 69 | 3.87E+04 | 8.25E−04 | 2.13E−08 | 14 |
| bsAb19147D | 547 ± 2.1 | 65 | 3.85E+04 | 7.91E−04 | 2.05E−08 | 15 |
| bsAb19148D | 565 ± 0.5 | 69 | 3.90E+04 | 7.98E−04 | 2.05E−08 | 14 |
| bsAb19149D | 566 ± 3.2 | 61 | 3.38E+04 | 6.22E−04 | 1.84E−08 | 19 |
| bsAb19150D | 544 ± 2.4 | 63 | 3.55E+04 | 6.91E−04 | 1.95E−08 | 17 |
| bsAb19151D | 525 ± 1.9 | 70 | 4.14E+04 | 7.60E−04 | 1.83E−08 | 15 |
| bsAb19152D | 568 ± 2 | 74 | 3.77E+04 | 7.87E−04 | 2.08E−08 | 15 |
| bsAb19153D | 557 ± 1.7 | 65 | 4.01E+04 | 7.91E−04 | 1.97E−08 | 15 |
| bsAb19154D | 540 ± 0.9 | 65 | 3.79E+04 | 7.29E−04 | 1.92E−08 | 16 |
| bsAb19155D | 536 ± 1.5 | 63 | 3.97E+04 | 7.92E−04 | 2.00E−08 | 15 |
| bsAb19156D | 539 ± 1 | 60 | 3.66E+04 | 8.42E−04 | 2.30E−08 | 14 |
| bsAb19757D | 543 ± 2.4 | 81 | 3.28E+04 | 6.25E−04 | 1.91E−08 | 18 |
| bsAb19758D | 519 ± 2.3 | 73 | 3.23E+04 | 6.63E−04 | 2.06E−08 | 17 |
| bsAb21236D | 68 ± 0.9 | 5 | 1.80E+04 | 1.31E−02 | 7.26E−07 | 0.9 |
| bsAb21237D | 269 ± 1.5 | 15 | 1.58E+04 | 4.42E−03 | 2.80E−07 | 2.6 |
| Isotype Control mAb | 540 ± 1.5 | −1 | NB* | NB* | NB* | NB* |

*NB indicates that no binding was observed under the current experimental conditions.

At 25° C., anti-LEPR/GP130 bispecific antibodies bound to hLEPR-MMH with K_{D} values ranging from 5.91 nM to 106 nM, as shown in Table 21. At 37° C., anti-LEPR monoclonal antibodies bound to hLEPR-MMH with K_{D} values ranging from 16.2 nM to 726 nM, as shown in Table 22.

TABLE 23
Binding kinetics parameters of mfLEPR-MMH binding
to anti-LEPR/GP130 bispecific antibodies at 25° C.

| | | 100 nM | | | | |
|---|---|---|---|---|---|---|
| | mAb | mfLEPR-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 417 ± 0.9 | 103 | 4.36E+04 | 1.28E−04 | 2.93E−09 | 90 |
| bsAb19140D | 442 ± 0.7 | 112 | 4.50E+04 | 1.21E−04 | 2.69E−09 | 95 |
| bsAb19141D | 457 ± 0.9 | 110 | 4.33E+04 | 1.24E−04 | 2.86E−09 | 93 |
| bsAb19142D | 450 ± 1.8 | 112 | 4.59E+04 | 1.26E−04 | 2.74E−09 | 92 |
| bsAb19143D | 445 ± 1.7 | 111 | 4.48E+04 | 1.36E−04 | 3.04E−09 | 85 |
| bsAb19144D | 455 ± 3 | 114 | 4.11E+04 | 1.24E−04 | 3.02E−09 | 93 |
| bsAb19145D | 436 ± 1.2 | 102 | 4.49E+04 | 1.23E−04 | 2.75E−09 | 94 |
| bsAb19146D | 460 ± 0.9 | 107 | 4.29E+04 | 1.13E−04 | 2.63E−09 | 102 |
| bsAb19147D | 437 ± 0.6 | 107 | 4.25E+04 | 1.19E−04 | 2.80E−09 | 97 |
| bsAb19148D | 452 ± 0.7 | 107 | 4.13E+04 | 1.19E−04 | 2.88E−09 | 97 |
| bsAb19149D | 446 ± 1 | 96 | 4.19E+04 | 1.11E−04 | 2.65E−09 | 104 |
| bsAb19150D | 449 ± 3.6 | 102 | 4.18E+04 | 1.17E−04 | 2.79E−09 | 99 |
| bsAb19151D | 418 ± 1.5 | 109 | 4.25E+04 | 1.17E−04 | 2.74E−09 | 99 |
| bsAb19152D | 454 ± 2.7 | 113 | 4.18E+04 | 1.24E−04 | 2.97E−09 | 93 |
| bsAb19153D | 440 ± 1.5 | 102 | 4.26E+04 | 1.26E−04 | 2.96E−09 | 92 |
| bsAb19154D | 436 ± 0.7 | 102 | 4.08E+04 | 1.21E−04 | 2.96E−09 | 96 |
| bsAb19155D | 429 ± 0.9 | 103 | 4.20E+04 | 1.22E−04 | 2.90E−09 | 95 |
| bsAb19156D | 439 ± 0.7 | 97 | 4.04E+04 | 1.32E−04 | 3.27E−09 | 87 |
| bsAb19757D | 429 ± 1.5 | 7 | IC^{#} | IC^{#} | IC^{#} | IC^{#} |
| bsAb19758D | 426 ± 3 | 9 | 3.86E+04 | 2.25E−02 | 5.83E−07 | 0.5 |
| bsAb21236D | 119 ± 0.3 | 12 | 5.79E+04 | 4.82E−03 | 8.34E−08 | 2.4 |
| bsAb21237D | 190 ± 0.6 | 16 | 3.08E+04 | 1.16E−03 | 3.78E−08 | 10 |
| Isotype Control | 426 ± 1.1 | 1 | NB* | NB* | NB* | NB* |

*NB indicates that no binding was observed under the current experimental conditions.
^{#}IC indicates that observed binding signal was less than three-fold above to the non-specific binding observed for isotype control antibody surface and/or the data cannot be used to measure binding kinetic parameters.

TABLE 24
Binding kinetics parameters of mfLEPR-MMH binding
to anti-LEPR/GP130 bispecific antibodies at 37° C.

| | | 100 nM | | | | |
|---|---|---|---|---|---|---|
| | mAb | mfLEPR-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 524 ± 1.2 | 163 | 1.03E+05 | 5.59E−04 | 5.42E−09 | 21 |
| bsAb19140D | 555 ± 0.8 | 175 | 1.02E+05 | 5.72E−04 | 5.60E−09 | 20 |
| bsAb19141D | 579 ± 1 | 173 | 1.03E+05 | 5.22E−04 | 5.05E−09 | 22 |
| bsAb19142D | 546 ± 3.5 | 172 | 9.49E+04 | 5.29E−04 | 5.57E−09 | 22 |
| bsAb19143D | 555 ± 1 | 171 | 9.59E+04 | 5.51E−04 | 5.74E−09 | 21 |
| bsAb19144D | 567 ± 1.5 | 178 | 9.91E+04 | 5.17E−04 | 5.22E−09 | 22 |
| bsAb19145D | 554 ± 1.3 | 162 | 1.03E+05 | 5.66E−04 | 5.48E−09 | 20 |
| bsAb19146D | 573 ± 1.6 | 164 | 1.00E+05 | 5.68E−04 | 5.68E−09 | 20 |
| bsAb19147D | 542 ± 1.3 | 163 | 1.00E+05 | 5.65E−04 | 5.64E−09 | 20 |
| bsAb19148D | 561 ± 1 | 163 | 9.25E+04 | 5.71E−04 | 6.17E−09 | 20 |
| bsAb19149D | 564 ± 1.8 | 154 | 6.25E+04 | 5.06E−04 | 8.09E−09 | 23 |
| bsAb19150D | 543 ± 3.4 | 158 | 9.89E+04 | 5.19E−04 | 5.25E−09 | 22 |
| bsAb19151D | 521 ± 1.2 | 167 | 1.05E+05 | 5.54E−04 | 5.30E−09 | 21 |
| bsAb19152D | 564 ± 1.7 | 169 | 9.99E+04 | 5.89E−04 | 5.90E−09 | 20 |
| bsAb19153D | 552 ± 1.2 | 158 | 6.58E+04 | 5.80E−04 | 8.81E−09 | 20 |
| bsAb19154D | 536 ± 1.2 | 159 | 9.60E+04 | 5.41E−04 | 5.64E−09 | 21 |
| bsAb19155D | 532 ± 1.3 | 155 | 1.03E+05 | 5.65E−04 | 5.49E−09 | 20 |
| bsAb19156D | 537 ± 1.4 | 148 | 9.64E+04 | 5.67E−04 | 5.88E−09 | 20 |
| bsAb19757D | 539 ± 1.3 | 7 | IC^{#} | IC^{#} | IC^{#} | IC^{#} |
| bsAb19758D | 517 ± 1.1 | 5 | IC^{#} | IC^{#} | IC^{#} | IC^{#} |
| bsAb21236D | 118 ± 1.3 | 9 | 6.19E+04 | 1.76E−02 | 2.84E−07 | 0.7 |
| bsAb21237D | 249 ± 0.6 | 27 | 2.81E+04 | 4.24E−03 | 1.51E−07 | 2.7 |
| Isotype Control | 536 ± 1.4 | 3 | NB* | NB* | NB* | NB* |

*NB indicates that no binding was observed under the current experimental conditions.
^{#}IC indicates that observed binding signal was less than three-fold above to the non-specific binding observed for isotype control antibody surface and/or the data cannot be used to measure binding kinetic parameters.

At 25° C., 21 out of 22 anti-LEPR/GP130 bispecific antibodies of the invention bound to mfLEPR-MMH with K_{D} values ranging from 2.63 nM to 583 nM, as shown in Table 23. At 37° C., 20 out of 22 anti-LEPR/GP130 bispecific antibodies of the invention bound to mfLEPR-MMH with K_{D} values ranging from 5.05 nM to 284 nM, as shown in Table 24.

TABLE 25
Binding kinetics parameters of hGP130-MMH binding
to anti-LEPR/GP130 bispecific antibodies at 25° C.

| | | 100 nM | | | | |
|---|---|---|---|---|---|---|
| | mAb | hGP130-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 168 ± 0.3 | 6 | IC^{#} | IC^{#} | IC^{#} | IC^{#} |
| bsAb19140D | 182 ± 0.3 | 23 | 4.55E+05 | 4.77E−02 | 1.05E−07 | 0.2 |
| bsAb19141D | 183 ± 0.5 | 15 | 1.06E+05 | 2.32E−02 | 2.19E−07 | 0.5 |
| bsAb19142D | 184 ± 0.8 | 31 | 1.73E+05 | 3.29E−03 | 1.90E−08 | 4 |
| bsAb19143D | 178 ± 0.6 | 16 | 3.20E+04 | 6.47E−04 | 2.02E−08 | 18 |
| bsAb19144D | 183 ± 1 | 27 | 6.43E+04 | 7.77E−04 | 1.21E−08 | 15 |
| bsAb19145D | 176 ± 0.3 | 19 | 3.41E+04 | 5.98E−04 | 1.75E−08 | 19 |
| bsAb19146D | 190 ± 0.6 | 31 | 6.09E+05 | 2.73E−04 | 4.48E−10 | 42 |
| bsAb19147D | 178 ± 0.3 | 7 | IC^{#} | IC^{#} | IC^{#} | IC^{#} |
| bsAb19148D | 186 ± 0.6 | 6 | IC^{#} | IC^{#} | IC^{#} | IC^{#} |
| bsAb19149D | 178 ± 0.5 | 7 | IC^{#} | IC^{#} | IC^{#} | IC^{#} |
| bsAb19150D | 183 ± 0.8 | 47 | 3.42E+05 | 4.80E−03 | 1.40E−08 | 2.4 |
| bsAb19151D | 168 ± 0.4 | 9 | 2.11E+04 | 2.21E−04 | 1.05E−08 | 52 |
| bsAb19152D | 184 ± 1.1 | 35 | 1.96E+05 | 1.13E−02 | 5.74E−08 | 1.0 |
| bsAb19153D | 179 ± 0.3 | 24 | 2.58E+05 | 4.19E−04 | 1.62E−09 | 28 |
| bsAb19154D | 180 ± 0.5 | 23 | 8.02E+04 | 2.04E−04 | 2.54E−09 | 57 |
| bsAb19155D | 176 ± 0.3 | 7 | IC^{#} | IC^{#} | IC^{#} | IC^{#} |
| bsAb19156D | 182 ± 0.4 | 27 | 8.95E+04 | 2.21E−03 | 2.47E−08 | 5 |
| bsAb19757D | 176 ± 0.4 | 16 | 1.12E+05 | 2.18E−02 | 1.96E−07 | 0.5 |
| bsAb19758D | 177 ± 1.2 | 51 | 3.13E+05 | 4.57E−03 | 1.46E−08 | 2.5 |
| bsAb21236D | 183 ± 0.4 | 12 | 3.03E+04 | 4.26E−03 | 1.40E−07 | 2.7 |
| bsAb21237D | 199 ± 0.5 | 17 | 4.96E+04 | 2.74E−03 | 5.52E−08 | 4 |
| Isotype Control | 175 ± 0.3 | 1 | NB* | NB* | NB* | NB* |

*NB indicates that no binding was observed under the current experimental conditions.
^{#}IC indicates that observed binding signal was less than three-fold above to the non-specific binding observed for isotype control antibody surface and/or the data cannot be used to measure binding kinetic parameters.

TABLE 26
Binding kinetics parameters of hGP130-MMH binding
to anti-LEPR/GP130 bispecific antibodies at 37° C.

| | | 100 nM | | | | |
|---|---|---|---|---|---|---|
| | mAb | hGP130-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 209 ± 0.4 | 3 | NB* | NB* | NB* | NB* |
| bsAb19140D | 230 ± 0.5 | 19 | 8.29E+05 | 1.20E−01 | 1.44E−07 | 0.1 |
| bsAb19141D | 229 ± 0.5 | 15 | 1.79E+05 | 6.21E−02 | 3.47E−07 | 0.2 |
| bsAb19142D | 226 ± 0.8 | 37 | 3.56E+05 | 6.22E−03 | 1.75E−08 | 1.9 |
| bsAb19143D | 219 ± 0.5 | 27 | 4.76E+04 | 2.12E−03 | 4.46E−08 | 5 |
| bsAb19144D | 231 ± 1.9 | 42 | 1.77E+05 | 1.88E−03 | 1.06E−08 | 6 |
| bsAb19145D | 223 ± 0.8 | 30 | 6.92E+04 | 2.22E−03 | 3.20E−08 | 5 |
| bsAb19146D | 240 ± 0.6 | 53 | 7.12E+05 | 6.41E−04 | 9.01E−10 | 18 |
| bsAb19147D | 217 ± 0.4 | 5 | IC^{#} | IC^{#} | IC^{#} | IC^{#} |
| bsAb19148D | 231 ± 0.6 | 8 | IC^{#} | IC^{#} | IC^{#} | IC^{#} |
| bsAb19149D | 222 ± 0.6 | 8 | IC^{#} | IC^{#} | IC^{#} | IC^{#} |
| bsAb19150D | 223 ± 1.2 | 52 | 4.33E+05 | 6.65E−03 | 1.54E−08 | 1.7 |
| bsAb19151D | 206 ± 0.4 | 16 | 2.24E+04 | 3.73E−04 | 1.66E−08 | 31 |
| bsAb19152D | 234 ± 0.8 | 28 | 4.07E+05 | 3.63E−02 | 8.91E−08 | 0.3 |
| bsAb19153D | 224 ± 0.3 | 33 | 3.19E+05 | 1.26E−03 | 3.95E−09 | 9 |
| bsAb19154D | 223 ± 0.3 | 37 | 2.69E+05 | 2.61E−04 | 9.73E−10 | 44 |
| bsAb19155D | 214 ± 0.6 | 4 | NB* | NB* | NB* | NB* |
| bsAb19156D | 222 ± 0.7 | 24 | 8.39E+04 | 7.97E−03 | 9.50E−08 | 1.4 |
| bsAb19757D | 216 ± 0.8 | 16 | 1.78E+05 | 6.35E−02 | 3.58E−07 | 0.2 |
| bsAb19758D | 216 ± 1.3 | 50 | 4.48E+05 | 7.47E−03 | 1.67E−08 | 1.5 |
| bsAb21236D | 220 ± 2.1 | 12 | 5.97E+04 | 1.48E−02 | 2.48E−07 | 0.8 |
| bsAb21237D | 263 ± 1 | 19 | 6.11E+04 | 8.79E−03 | 1.44E−07 | 1.3 |
| Isotype Control | 220 ± 0.7 | 4 | NB* | NB* | NB* | NB* |

*NB indicates that no binding was observed under the current experimental conditions.
^{#}IC indicates that observed binding signal was less than three-fold above to the non-specific binding observed for isotype control antibody surface and/or the data cannot be used to measure binding kinetic parameters.

At 25° C., 17 out of 22 anti-LEPR/GP130 bispecific antibodies of the invention bound to hGP130-MMH with K_{D} values ranging from 448 pM to 219 nM, as shown in Table 25. At 37° C., 17 out of 22 anti-LEPR/GP130 bispecific antibodies bound to hGP130-MMH with K_{D} values ranging from 901 pM to 358 nM, as shown in Table 26.

TABLE 27
Binding kinetics parameters of mfGP130-MMH binding
to anti-LEPR/GP130 bispecific antibodies at 25° C.

| | | 100 nM | | | | |
|---|---|---|---|---|---|---|
| | mAb | mfGP130-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 168 ± 0.3 | 6 | IC^{#} | IC^{#} | IC^{#} | IC^{#} |
| bsAb19140D | 182 ± 0.5 | 20 | 4.02E+05 | 5.46E−02 | 1.36E−07 | 0.2 |
| bsAb19141D | 183 ± 0.6 | 15 | 1.12E+05 | 2.07E−02 | 1.84E−07 | 0.6 |
| bsAb19142D | 183 ± 0.9 | 28 | 1.20E+05 | 3.74E−03 | 3.12E−08 | 3.1 |
| bsAb19143D | 177 ± 0.7 | 10 | 3.01E+04 | 1.30E−03 | 4.30E−08 | 9 |
| bsAb19144D | 183 ± 1.3 | 27 | 5.67E+04 | 7.62E−04 | 1.34E−08 | 15 |
| bsAb19145D | 175 ± 0.3 | 18 | 3.31E+04 | 5.76E−04 | 1.74E−08 | 20 |
| bsAb19146D | 190 ± 0.4 | 31 | 5.02E+05 | 2.58E−04 | 5.13E−10 | 45 |
| bsAb19147D | 177 ± 0.3 | 5 | IC^{#} | IC^{#} | IC^{#} | IC^{#} |
| bsAb19148D | 186 ± 0.6 | 6 | IC^{#} | IC^{#} | IC^{#} | IC^{#} |
| bsAb19149D | 178 ± 0.8 | 6 | IC^{#} | IC^{#} | IC^{#} | IC^{#} |
| bsAb19150D | 183 ± 1 | 44 | 3.08E+05 | 5.19E−03 | 1.68E−08 | 2.2 |
| bsAb19151D | 168 ± 0.5 | 3 | NB* | NB* | NB* | NB* |
| bsAb19152D | 184 ± 0.7 | 52 | 2.95E+05 | 5.53E−03 | 1.88E−08 | 2.1 |
| bsAb19153D | 178 ± 0.4 | 23 | 2.29E+05 | 3.96E−04 | 1.73E−09 | 29 |
| bsAb19154D | 180 ± 0.5 | 22 | 7.23E+04 | 2.02E−04 | 2.79E−09 | 57 |
| bsAb19155D | 175 ± 0.3 | 7 | 9.85E+04 | 1.84E−02 | 1.86E−07 | 0.6 |
| bsAb19156D | 182 ± 0.4 | 30 | 7.33E+04 | 1.62E−03 | 2.21E−08 | 7 |
| bsAb19757D | 175 ± 0.5 | 15 | 1.08E+05 | 2.04E−02 | 1.88E−07 | 0.6 |
| bsAb19758D | 176 ± 1.7 | 46 | 3.07E+05 | 5.07E−03 | 1.65E−08 | 2.3 |
| bsAb21236D | 240 ± 0.6 | 21 | 2.94E+04 | 3.05E−03 | 1.04E−07 | 3.8 |
| bsAb21237D | 212 ± 0.6 | 18 | 3.98E+04 | 2.13E−03 | 5.34E−08 | 5 |
| Isotype Control | 175 ± 0.5 | 1 | NB* | NB* | NB* | NB* |

*NB indicates that no binding was observed under the current experimental conditions.
^{#}IC indicates that observed binding signal was less than three-fold above to the non-specific binding observed for isotype control antibody surface and/or the data cannot be used to measure binding kinetic parameters.

TABLE 28
Binding kinetics parameters of mfGP130-MMH binding
to anti-LEPR/GP130 bispecific antibodies at 37° C.

| | | 100 nM | | | | |
|---|---|---|---|---|---|---|
| | mAb | mfGP130-MMH | | | | |
| Bispecific | Capture | Bound | kₐ | k_{d} | K_{D} | t½ |
| Antibody | Level (RU) | (RU) | (1/Ms) | (1/s) | (M) | (min) |
| bsAb19139D | 208 ± 0.4 | 2 | NB* | NB* | NB* | NB* |
| bsAb19140D | 229 ± 0.5 | 14 | 7.97E+05 | 1.46E−01 | 1.83E−07 | 0.1 |
| bsAb19141D | 228 ± 0.5 | 15 | 2.42E+05 | 5.13E−02 | 2.12E−07 | 0.2 |
| bsAb19142D | 226 ± 1.4 | 33 | 3.32E+05 | 7.15E−03 | 2.16E−08 | 1.6 |
| bsAb19143D | 217 ± 1.1 | 14 | 6.83E+04 | 3.96E−03 | 5.79E−08 | 2.9 |
| bsAb19144D | 230 ± 1.3 | 41 | 1.68E+05 | 1.83E−03 | 1.09E−08 | 6 |
| bsAb19145D | 222 ± 0.5 | 29 | 5.88E+04 | 2.21E−03 | 3.76E−08 | 5 |
| bsAb19146D | 239 ± 0.5 | 53 | 6.53E+05 | 6.26E−04 | 9.59E−10 | 18 |
| bsAb19147D | 216 ± 0.4 | 3 | NB* | NB* | NB* | NB* |
| bsAb19148D | 231 ± 0.5 | 8 | IC^{#} | IC^{#} | IC^{#} | IC^{#} |
| bsAb19149D | 221 ± 0.8 | 8 | IC^{#} | IC^{#} | IC^{#} | IC^{#} |
| bsAb19150D | 222 ± 1.3 | 46 | 4.01E+05 | 7.73E−03 | 1.93E−08 | 1.5 |
| bsAb19151D | 209 ± 7.1 | 6 | NB* | NB* | NB* | NB* |
| bsAb19152D | 233 ± 1 | 51 | 3.42E+05 | 1.77E−02 | 5.16E−08 | 0.7 |
| bsAb19153D | 223 ± 0.3 | 32 | 2.84E+05 | 1.23E−03 | 4.33E−09 | 9 |
| bsAb19154D | 223 ± 0.4 | 35 | 2.19E+05 | 3.43E−04 | 1.57E−09 | 34 |
| bsAb19155D | 213 ± 0.4 | 3 | NB* | NB* | NB* | NB* |
| bsAb19156D | 221 ± 0.6 | 26 | 8.75E+04 | 7.67E−03 | 8.76E−08 | 1.5 |
| bsAb19757D | 215 ± 0.9 | 14 | 1.33E+05 | 5.98E−02 | 4.50E−07 | 0.2 |
| bsAb19758D | 215 ± 1 | 45 | 4.02E+05 | 7.30E−03 | 1.82E−08 | 1.6 |
| bsAb21236D | 285 ± 2.1 | 18 | 6.98E+04 | 1.25E−02 | 1.79E−07 | 0.9 |
| bsAb21237D | 270 ± 1 | 21 | 3.69E+04 | 7.92E−03 | 2.15E−07 | 1.5 |
| Isotype Control | 219 ± 0.5 | 4 | NB* | NB* | NB* | NB* |

*NB indicates that no binding was observed under the current experimental conditions.
^{#}IC indicates that observed binding signal was less than three-fold above to the non-specific binding observed for isotype control antibody surface and/or the data cannot be used to measure binding kinetic parameters.

At 25° C., 17 out of 22 LEPR x GP130 bispecific antibodies of the invention bound to mfGP130-MMH with K_{D} values ranging from 513pnM to 188 nM, as shown in Table 27. At 37° C., 16 out of 22 LEPR x GP130 bispecific antibodies of the invention bound to mfGP130-MMH with K_{D} values ranging from 959 pM to 450 nM, as shown in Table 28.

### Example 11: LEPR x GP130 Bispecific Antibody Cell Binding Measured by FACS Analysis

In order to assess cell binding by LEPRxGP130 bispecific antibodies, HEK293 stable cell lines were generated. One cell line was generated to stably over-express full length human GP130 (amino acids 1-918 of accession #P40189 with leucine at position 2 changed to valine, a natural variant) along with a luciferase reporter (Stat3-luciferase, Stat3-luc, SA Bioscience, #CLS-6028L), and was sorted twice using flow cytometry for high expression of GP130. This cell line is referred to hereafter as “HEK293/Stat3-luc/gp130-2 X Sort.” Another cell line used in this Example, known hereafter as “HEK293/hLEPR-GPI,” stably expresses the extracellular domain of human LEPR (amino acids 22-839 of accession #P48357, Isoform B) with an N-terminal myc-myc tag and C-terminal peptide sequence from human carboxypeptidase M that guides the addition of GPI (Glycosylphosphatidylinositol) such that the protein can be GPI-anchored to the membrane.

For the FACS analysis, 0.5×106 cells/well of HEK293 parental cells, HEK293/Stat3-luc/gp130-2 X Sort cells, and HEK293/hLEPR-GPI cells, were incubated with 200 nM of the conventional antibodies against either GP130 or LEPR or with LEPRxGP130 bispecific antibodies, along with isotype control antibodies at 4° C. in PBS (without calcium and magnesium) containing 2% FBS.

To test whether the anti-LEPR antibody binding or LEPRxGP130 bispecific antibody binding to cells was affected by the presence of Leptin, 1 μM human Leptin (R&D Systems, #398-LP) was incubated with the cells for 30 minutes, followed by the addition of anti-LEPR antibodies or isotype control antibody. After incubation with primary antibodies, the cells were stained with 8 mg/mL of Alexa Fluor®-647 conjugated secondary antibody (Jackson ImmunoResearch Laboratories Inc., #109-607-003) for 30 minutes. Cells were fixed using BD CytoFix™ (Becton Dickinson, #554655) and analyzed on an IQue® (Intellicyt) Flow Cytometer. Unstained and secondary antibody alone controls were also tested for all cell lines. The results were analyzed using ForeCyt® (IntelliCyt) and FlowJo version 10 softwares to determine the geometric means of fluorescence for viable cells.

As shown in Table 29, two LEPRxGP130 bispecific antibodies of the invention tested at 200 nM demonstrated binding to HEK293/gp130 2 X Sort cells with binding ratios of 132- and 169-fold and binding to HEK293/hLEPR-GPI cells with binding ratios of 4423- and 6320-fold without Leptin, and 3596- and 5932-fold in the presence of 1 μM Leptin. The GP130-binding arm of the bispecific antibodies of the invention made as a conventional antibody (mAb16683) demonstrated binding to HEK293/gp130 2 X Sort cells with a binding ratio of 235-fold and binding to HEK293/hLEPR-GPI cells with a binding ratio of 21-fold. The LEPR binding arms of the bispecifics (mAb18445 and mAb18446) made as a conventional antibody demonstrated binding to HEK293/hLEPR-GPI cells with binding ratios of 4711- and 7023-fold without Leptin, and 4246- and 6390-fold in the presence of 1 μM Leptin. The anti-GP130 and anti-LEPR conventional and bispecific antibodies demonstrated binding to the HEK293 parental cells with binding ratios ranging from 3- to 24-fold. The isotype control antibodies and secondary antibodies alone samples also did not demonstrate significant binding to any of the cell lines tested with or without Leptin, with binding ratios ranging from 1- to 3-fold.

TABLE 29
Antibody Binding to Cells Assessed by FACS

| | Binding Ratio: | | | |
|---|---|---|---|---|
| | Normalized to Unstained Sample of Each Cell Line (FL4-A) | | | |
| | | | HEK293/hLEPR- | HEK293/hLEPR- |
| | HEK293 | HEK293/ | GPI cells (No | GPI cells (1 μM |
| Antibody | parental | gp130 2X Sort | Leptin) | Leptin) |
| bsAb21236 | 13 | 132 | 4423 | 3596 |
| (LEPR × GP130) | | | | |
| bsAb21237 | 24 | 169 | 6320 | 5932 |
| (LEPR × GP130) | | | | |
| mAb16683 | 17 | 235 | 21 | Not Tested |
| (anti-GP130 mAb) | | | | |
| mAB18445 | 3 | Not Tested | 4711 | 4246 |
| (anti-LEPR mAb) | | | | |
| mAB18446 | 6 | Not Tested | 7023 | 6390 |
| (anti-LEPR mAb) | | | | |
| Isotype control | 2 | 2 | 3 | 3 |
| antibody | | | | |
| Secondary antibody | 1 | 1 | 3 | 2 |
| alone | | | | |
| No Antibody | 1 | 1 | 1 | Not Tested |

### Example 12: Functional Cell-Based Assays

The cytokine receptors GP130 (amino acids 1-918 of accession #P40189) and LEPR (amino acids 1-1165 of accession #P48357) have a non-covalently associated tyrosine kinase, JAK2, bound to the membrane proximal region of their cytoplasmic domains. Treatment with cognate ligand or agonist antibody culminates in activation of JAK2, which in turn phosphorylates key tyrosine residues on the cytoplasmic region of the receptor. The phosphorylated tyrosine residues serve as docking sites for signaling complexes that upon phosphorylation lead to stimulation of signaling pathways such as STAT3 and ERK. For the LEPR, tyrosine residue Y1141 mediates STAT3 signaling and mutation of this residue to phenylalanine (Y1141 F) eliminates STAT3 signaling (Carpenter et al., 1998, Proc. Natl. Acad. Sci. USA 95:6061-6066).

A bioassay was developed to detect the transcriptional activation of STAT3 via the promotion of GP130 and LEPR heterodimerization following treatment with LEPR x GP130 bsAbs. In particular, a reporter cell line that stably expresses mutant human LEPR (Y1141F) and wild-type human GP130, along with a STAT3 responsive luciferase reporter (STAT3-Luc; Qiagen CLS-6028L) was generated. The resulting stable cell line, referred to as HEK293.STAT3.Luc.GP130.hLEPR (Y1141F), was isolated and maintained in DME medium supplemented with 10% FBS, 1 ug/mL Puromycin, 250 ug/mL of Hygromycin B, 500 ug/mL of G418 and Penicillin/Streptomycin/L-Glutamine. Two LEPRxGP130 bispecific antibodies were identified in this bioassay, bsAb21236 and bsAb21237, which promoted STAT3 activity in the presence of leptin.

For the bioassay, HEK293.STAT3.Luc.GP130.hLEPR (Y1141F) cells were plated at a density of 20,000 cells/well and then the following day the media was replaced with 80 uL of Opti-MEM supplemented with 1% BSA and 0.1% FBS (Assay Buffer). Subsequently, 10 uL of fixed-concentration of 10 nM of human Leptin (hLeptin; R&D Systems, #398-LP-01M) was added to the wells. Immediately following the hLeptin treatment, the bispecific antibodies were half-log serially diluted (12 points) to final concentrations ranging from 500 nM to 5 pM in Assay Buffer and were then added to the cells. The isotype control and human OSM (hOSM; R&D Systems, #295-OM/CF) were half-log diluted (11 points) to final concentrations ranging from 100 nM to 1 pM in Assay Buffer and were then added to the cells. The plates were then placed in the incubator overnight at 37° C. in 5% CO₂. One-Glo reagent (Promega, #E6051) was then added to the samples and luciferase activity was measured on Envision Multilable Plate Reader (Perkin Elmer) in Luminescent mode. The relative light units (RLU) values were obtained and the results were analyzed using nonlinear regression with GraphPad Prism software (Graph Pad). The maximum RLU value obtained from the hOSM dose response was defined as 100% activation in the HEK293.STAT3.Luc.GP130.hLepR (Y1141F) cell-based assay.

The ability of LEPRxGP130 bispecific antibodies to activate via GP130-mediated cell signaling was evaluated in the HEK293.STAT3.Luc.gp130.hLepR (Y1141F) cell-based assay and the resulting EC₅₀ values and percentage activation are shown in Table 30. The responsiveness of the cell line was confirmed using a dose response of hOSM, which demonstrated activation in the assay with an EC₅₀ value of 592 pM. Both LEPRxGP130 bispecific antibodies tested, bsAb21236 and bsAb21237, demonstrated activation in this assay with EC₅₀ values of 2.11 nM and 2.46 nM, respectively. Both bispecific antibodies have approximately 20% of maximal activation observed with hOSM.

TABLE 30
Activation of HEK293.STAT3.Luc.GP130.hLEPR (Y1141F) Cell
line by LEPR × GP130 Bispecific Antibodies

| | | Percentage activation as |
|---|---|---|
| Molecule tested | EC₅₀ (M) | compared to hOSM |
| bsAb21236 | 2.109E−09 | 25% |
| bsAb21237 | 2.460E−09 | 20% |
| hOSM | 5.919E−10 | 100% |
| Isotype control | No activation | No activation |
| antibody | | |

To confirm that the activation by LEPRxGP130 bispecific antibodies in the HEK293.STAT3.Luc.GP130.hLEPR (Y1141F) cell-based assay was due to activation through both LEPR and GP130, a competition bioassay was performed using soluble LEPR and GP130 proteins to block the activation by the bispecific antibodies. The competition bioassay utilized an excess fixed concentration, 500 nM, of the extracellular domain of human LEPR with a C-terminal hFc tag (hLEPR-hFc; SEQ ID NO:189), the extracellular domain of human LEPR with a C-terminal myc-myc-hexahistidine tag (hLEPR-MMH; SEQ ID NO:187), the extracellular domain of human GP130 with a C-terminal hFc tag (hGP130-hFc; SEQ ID NO:197), and the extracellular domain of human CNTFR with a C-terminal myc-myc-hexahistidine tag (hCNTFR-MMH; SEQ ID NO:198).

For the assay, HEK293.STAT3.Luc.GP130.hLEPR (Y1141F) cells were plated at the density of 20,000 cells/well and then the following day the media was replaced with 70 uL of Opti-MEM supplemented with 1% BSA and 0.1% FBS (Assay Buffer). 10 uL of fixed-concentration of 10 nM of human Leptin (hLeptin; R&D Systems, #398-LP-01M) was added to the wells. Immediately following the hLeptin treatment, 10 nM of the bispecific antibodies in Assay Buffer were then added to the cells. Immediately after, an excess amount, 500 nM, of the soluble proteins hLEPR-hFc, hLEPR-MMH, hGP130-hFc, and hCNTFR-MMH were added to the appropriate designated wells. The plates were then placed in the incubator overnight at 37° C. in 5% CO₂. One-Glo reagent (Promega, #E6051) was then added to the samples and luciferase activity was measured on Envision Multilable Plate Reader (Perkin Elmer) in Luminescent mode. The relative light units (RLU) values were obtained and the results were analyzed using GraphPad Prism software (GraphPad).

The competition assay result demonstrated that soluble hLEPR-hFc, hLEPR-MMH and hGP130-hFc were able to block the bispecific antibody activity whereas soluble hCNTFR.mmh did not block the activity of the bispecific antibodies. The activity of the bispecific antibodies alone is defined as 100% whereas the activity of isotype control represents 0% activity.

Table 31, below shows activation of HEK293.STAT3.Luc.GP130.hLEPR cells by bispecific antibodies in the presence of soluble human LEPR, GP130 and CNTFR. Table 32 shows RLU production in the presence of hLEPR-MMH, hLEPR-hFc, hGP130-hFc or hCNTFR-MMH.

TABLE 31
Activation of HEK293.STAT3.Luc.GP130.hLEPR (Y1141F) cells by LEPR × GP130
bispecific antibodies in the presence of soluble human LEPR, GP130, and CNTFR

| | RLU in the | | RLU in the | | RLU in the | | RLU in the | | | |
|---|---|---|---|---|---|---|---|---|---|---|
| | presence of | | presence of | | presence of | | presence of | | | |
| Bispecific | hLEPR-MMH | | hLEPR-hFc | | hGP130-hFc | | hCNTFR-MMH | | | |
| Antibody | [500 nM] | | [500 nM] | | [500 nM] | | [500 nM] | | Assay Buffer | |
| bsAb21236 | 12840 | 13160 | 11640 | 10320 | 15520 | 13840 | 48560 | 43080 | 48920 | 42160 |
| bsAb21237 | 11280 | 11800 | 10480 | 9480 | 14320 | 15680 | 39920 | 43400 | 44920 | 38080 |
| Isotype control | | | | | | | | | 12160 | 12080 |

TABLE 32
Activation of HEK293.STAT3.Luc.GP130.hLEPR (Y1141F) cells by LEPR × GP130
bispecific antibodies in the presence of soluble human LEPR, GP130, and CNTFR

| | RLU in the | RLU in the | RLU in the | RLU in the | |
|---|---|---|---|---|---|
| | presence of | presence of | presence of | presence of | |
| Bispecific | hLEPR-MMH | hLEPR-hFc | hGP130-hFc | hCNTFR-MMH | |
| Antibody | [500 nM] | [500 nM] | [500 nM] | [500 nM] | Assay Buffer |
| bsAb21236 | 3% | −3% | 8% | 101% | 100% |
| bsAb21237 | −2% | −7% | 10% | 101% | 100% |
| Isotype | | | | | 0% |
| control | | | | | |

### Example 13: In Vivo Efficacy of LEPR x GP130 Bispecific Antibodies bsAb21236 and bsAb21237 in Diet-Induced Obese Mice

The effects of two LEPR x GP130 bispecific antibodies of the invention, bsAb21236 and bsAb21237, on body weight were determined in an in vivo model using high fat diet fed obese LEPRHu/Hu;IL6STHu/Hu mice, that express a leptin receptor composed of the human LEPR extracellular domain sequence in place of the murine LEPR extracellular domain sequence and a GP130 protein composed of the human IL6ST extracellular domain sequence in place of the murine IL6ST extracellular domain sequence.

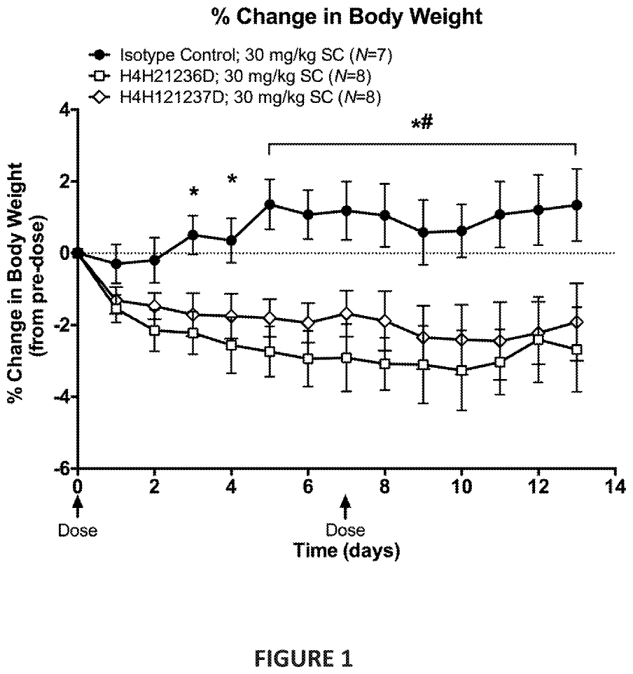

On day 0, twenty-three male LEPRHu/Hu;IL6STHu/Hu mice that were fed a high fat diet for 12 weeks were randomized into three groups of 7 to 8 mice based on body weight. On day 0 and 7, each group received via subcutaneous injection a dose of either isotype control antibody at 30 mg/kg, bsAb21236 at 30 mg/kg, or bsAb21237 at 30 mg/kg. The isotype control antibody used does not bind any known mouse protein. The body weight of each mouse was measured daily for the duration of the study. The percent change in body weight from day 0 was calculated for each animal at every time point measured. FIG. 1 summarizes the average percent change in body weight for animals in each treatment group. All results are expressed as mean±SEM.

As shown in FIG. 1, LEPRHu/Hu;IL6STHu/Hu mice treated with bsAb21236 at 30 mg/kg exhibited significant reductions in percent body weight change starting at three days post antibody treatment and at the other subsequent time points measured compared to mice injected with isotype control antibody. LEPRHu/Hu;IL6STHu/Hu mice treated with bsAb21237 at 30 mg/kg exhibited a significant reduction in percent body weight change starting at five days post antibody treatment and at the other subsequent time points measured compared to mice injected with isotype control antibody.

### Example 14: Transcriptional Activation of STAT3 Via the Promotion of GP130 and LEPR (Short Form) Heterodimerization Following Treatment with LEPR x GP130 bsAbs

GP130 serves a co-receptor for multiple cytokines and is expressed broadly in human tissues (Taga T., Kishimoto T. gp130 and the interleukin-6 family of cytokines. Annu. Rev. Immunol 1997; 15:797-819). Isoforms of the LEPR are generated through alternative splicing, resulting a long isoform b (LEPR-b) and several short forms, including isoform a (LEPR-a) which shows the highest and broadest expression pattern (Tartaglia LA. The leptin receptor. J Biol Chem 1997; 272: 6093-6096). All the isoforms share the same extracellular domain, transmembrane region and a short stretch of the cytoplasmic domain, containing the Box 1 region, followed by a variable region. The long form contains intracellular sequence motifs required for mediating all the signaling capabilities of leptin whereas the short forms are lacking these regions. Since the extracellular domain of the short forms is identical to the signaling competent long form, the bispecific antibodies can bind to the short forms and generate complexes with GP130. The primary intended target tissue for LEPR agonists in general (including LEPRxGP130 bispecific antigen binding molecules) is the brain where LEPR isoform b is predominantly expressed. Given the broad expression of GP130 and isoform a of LEPR, however, there existed the potential for unwanted STAT3 activation in tissues such as the liver.

In order to evaluate signaling outcomes resulting from complexing of LEPR short isoform a and GP130, a bioassay was developed to detect the transcriptional activation of STAT3 via the promotion of GP130 and LEPR (short form) heterodimerization following treatment with LEPR x GP130 bsAbs. In particular, a reporter cell line that stably expresses the dominant short form of LEPR (NP 001003679.1), to be referred to as hLEPR(a), and wild-type human GP130, along with a STAT3 responsive luciferase reporter (STAT3-Luc; Qiagen CLS-6028L) was generated. The resulting stable cell line, referred to as HEK293.STAT3.Luc.GP130.hLEPR(a), was isolated and maintained in DME medium supplemented with 10% FBS, 1 ug/mL Puromycin, 250 ug/mL of Hygromycin B, 500 ug/mL of G418 and Penicillin/Streptomycin/L-Glutamine.

For the bioassay, HEK293.STAT3.Luc.GP130.hLEPR (a) cells were plated at a density of 20,000 cells/well and then the following day the media was replaced with 80 uL of Opti-MEM supplemented with 1% BSA and 0.1% FBS (Assay Buffer). Subsequently, 10 uL of fixed-concentration of 10 nM of human Leptin (hLeptin; R&D Systems, #398-LP-01 M) was added to the wells. Immediately following the hLeptin treatment, the bispecific antibodies were half-log serially diluted (12 points) to final concentrations ranging from 500 nM to 5 pM in Assay Buffer and were then added to the cells. As controls, human leptin and OSM (hOSM; R&D Systems, #295-OM/CF) were half-log diluted (11 points) to final concentrations ranging from 100 nM to 1 pM in Assay Buffer and were then added to the cells. The plates were then placed in the incubator overnight at 37° C. in 5% CO₂. One-Glo reagent (Promega, #E6051) was then added to the samples and luciferase activity was measured on Envision Multilable Plate Reader (Perkin Elmer) in Luminescent mode. The relative light units (RLU) values were obtained and the results were analyzed using nonlinear regression with GraphPad Prism software (Graph Pad). The maximum RLU value obtained from the hOSM dose response was defined as 100% activation in the HEK293.STAT3.Luc.GP130.hLEPR (a) cell-based assay.

The ability of LEPR x GP130 bispecific antibodies to activate via GP130-mediated cell signaling was evaluated in the HEK293.STAT3.Luc.gp130.hLEPR (a) cell-based assay and the resulting responses are shown in Table 33. The responsiveness of the cell line was confirmed using a dose response of hOSM, which demonstrated activation in the assay with an EC₅₀ value of 121 pM and its maximum response was designated as 100% activation. Both LEPR x GP130 bispecific antibodies tested, bsAb21236 and bsAb21237, failed to activate STAT3 signaling with LEPR(a). Similar to leptin, the bispecific antigen-binding proteins of the present invention generate productive STAT3 signaling only in cell types containing the long form of LEPR.

TABLE 33
Activation of HEK293.STAT3.Luc.GP130.hLEPR (a) Cell line
by LEPR × GP130 Bispecific Antibodies

| | | Percentage maximum |
|---|---|---|
| | | activation as compared |
| Molecule tested | EC₅₀ (M) | to hOSM |
| bsAb21236 + 10 nM | No activation | No activation |
| leptin | | |
| bsAb21237 + 10 nM | No activation | No activation |
| leptin | | |
| hOSM | 1.21E−10 | 100% |
| hLeptin | No activation | No activation |

In summary, the data shows that the LEPRxGP130 bispecific antibodies provided herein do not activate signaling through the “short form” of the leptin receptor (LEPR-a isoform), but do activate signaling through the “long form” of the leptin receptor (LEPR-b isoform). The relevance of this finding is that it suggests that these bispecific antibodies will exert their activity primarily in the brain where the ‘b’ isoform is predominantly expressed, but not in other tissues such as the liver where the ‘a’ form is broadly expressed. Given that these bispecific antibodies can be used to treat obesity by activating LEPR signaling in the brain, this work confirms that the bispecific antibodies provided herein are effective at targeting leptin signaling where it is needed (in the brain) while avoiding unwanted signaling elsewhere in the body (such as the liver, e.g. in inflammatory hepatocellular adenoma (see Rebouissou et al., Nature Letters, 457(8): 200-205, 2009)).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

TABLE 34
Informal Sequence Listing

| SEQ | | |
|---|---|---|
| ID | | |
| NO | Sequence | Description |
| 1. | caggtgcagc tggtggagtc cgggggaggc gtggtccagc ctgggaggtc cctgagactc | DNA |
| | tcctgtacag cgtctggatt caccttcagt agttatgcca tgtactgggt ccgccaggct | nucleotide |
| | ccaggcaagg ggctggagtg ggtgtcagtt atatactatg atggaagtta taaatactat | sequence |
| | gcagactccg tgaagggccg attcaccatc tccagagaca attccaagaa cacgctgtat | |
| | ctgcaaatgg acagcctgag agccgaggac acggctgtct attactgtgc gagttataac | |
| | tggaactact ggtacttcga tttctggggc cgtggcaccc tggtcactgt ctcctca | |
| 2. | QVQLVESGGG VVQPGRSLRL SCTASGFTFS SYAMYWVRQA PGKGLEWVSV IYYDGSYKYY | AA amino |
| | ADSVKGRFTI SRDNSKNTLY LQMDSLRAED TAVYYCASYN WNYWYFDFWG RGTLVTVSS | acid |
| | | sequence |
| 3. | ggattcacct tcagtagtta tgcc | DNA |
| | | nucleotide |
| | | sequence |
| 4. | GFTFSSYA | AA amino |
| | | acid |
| | | sequence |
| 5. | atatactatg atggaagtta taaa | DNA |
| | | nucleotide |
| | | sequence |
| 6. | IYYDGSYK | AA amino |
| | | acid |
| | | sequence |
| 7. | gcgagttata actggaacta ctggtacttc gatttc | DNA |
| | | nucleotide |
| | | sequence |
| 8. | ASYNWNYWYF DF | AA amino |
| | | acid |
| | | sequence |
| 9. | gacatccaga tgacccagtc tccatcctcc ctgtctgcat ctgtaggaga cagagtcacc | DNA |
| | atcacttgcc gggcaagtca gagcattagc agctatttaa attggtatca gcagaaacca | nucleotide |
| | gggaaagccc ctaagctcct gatctatgct gcatccagtt tgcaaagtgg ggtcccgtca | sequence |
| | aggttcagtg gcagtggatc tgggacagat ttcactctca ccatcagcag tctgcaacct | |
| | gaagattttg caacttacta ctgtcaacag agttacagta cccctccgat caccttcggc | |
| | caagggacac gactggagat taaa | |
| 10. | DIQMTQSPSS LSASVGDRVT ITCRASQSIS SYLNWYQQKP GKAPKLLIYA ASSLQSGVPS | AA amino |
| | RFSGSGSGTD FTLTISSLQP EDFATYYCQQ SYSTPPITFG QGTRLEIK | acid |
| | | sequence |
| 11. | cagagcatta gcagctat | DNA |
| | | nucleotide |
| | | sequence |
| 12. | QSISSY | AA amino |
| | | acid |
| | | sequence |
| 13. | gctgcatcc | DNA |
| | | nucleotide |
| | | sequence |
| 14. | AAS | AA amino |
| | | acid |
| | | sequence |
| 15. | caacagagtt acagtacccc tccgatcacc | DNA |
| | | nucleotide |
| | | sequence |
| 16. | QQSYSTPPIT | AA amino |
| | | acid |
| | | sequence |
| 17. | caggtgcagc tggtggagtc tgggggaggc gtggtccagc cgggggggtc cctgagactc | DNA |
| | tcctgtgcag cctctggatt caccttcagt acttatgaca ttcactgggt ccgccaggct | nucleotide |
| | ccaggcaagg ggctggagtg ggtggcagtc atatcttttg atggaagtaa tagaaattat | sequence |
| | ggagactccg tgaagggccg attcaccatc tccagggaca attccgacaa taccctgttt | |
| | ctggaaatga acaatctgag atttgaagac acggctgtgt attactgtgc gaaagagggc | |
| | tacggtgttg acttccaaca ctggggccag ggcaccctgg tcaccgtctc ctca | |
| 18. | QVQLVESGGG VVQPGGSLRL SCAASGFTFS TYDIHWVRQA PGKGLEWVAV ISFDGSNRNY | AA amino |
| | GDSVKGRFTI SRDNSDNTLF LEMNNLRFED TAVYYCAKEG YGVDFQHWGQ GTLVTVSS | acid |
| | | sequence |
| 19. | ggattcacct tcagtactta tgac | DNA |
| | | nucleotide |
| | | sequence |
| 20. | GFTFSTYD | AA amino |
| | | acid |
| | | sequence |
| 21. | atatcttttg atggaagtaa taga | DNA |
| | | nucleotide |
| | | sequence |
| 22. | ISFDGSNR | AA amino |
| | | acid |
| | | sequence |
| 23. | gcgaaagagg gctacggtgt tgacttccaa cac | DNA |
| | | nucleotide |
| | | sequence |
| 24. | AKEGYGVDFQ H | AA amino |
| | | acid |
| | | sequence |
| 25. | caggtgcagc tgcaggagtc gggcccagga ctggtgaagc cttcggagac cctgtccctc | DNA |
| | acctgcactg tctctggtgg ctccatcagt aattactact ggacctggat ccggcagccc | nucleotide |
| | ccagggaagg gactggagtg gattgggcat atctctaaca gtgggaccac caactataac | sequence |
| | ccctccctca agagtcgagt caccatatca gtagacacgt ccaagaacca gttctccctg | |
| | aagctgacct ctgtgaccgc cgcagacacg gccgtgtatt actgtgcgag actccccctc | |
| | ggtattgcag cagttgatac agattattat tattacttcg gtatggacgt ctggggccaa | |
| | gggaccacgg tcaccgtctc ctca | |
| 26. | QVQLQESGPG LVKPSETLSL TCTVSGGSIS NYYWTWIRQP PGKGLEWIGH ISNSGITNYN | AA amino |
| | PSLKSRVTIS VDTSKNQFSL KLTSVTAADT AVYYCARLPL GIAAVDTDYY YYFGMDVWGQ | acid |
| | GTTVTVSS | sequence |
| 27. | ggtggctcca tcagtaatta ctac | DNA |
| | | nucleotide |
| | | sequence |
| 28. | GGSISNYY | AA amino |
| | | acid |
| | | sequence |
| 29. | atctctaaca gtgggaccac c | DNA |
| | | nucleotide |
| | | sequence |
| 30. | ISNSGTT | AA amino |
| | | acid |
| | | sequence |
| 31. | gcgagactcc ccctcggtat tgcagcagtt gatacagatt attattatta cttcggtatg | DNA |
| | gacgtc | nucleotide |
| | | sequence |
| 32. | ARLPLGIAAV DTDYYYYFGM DV | AA amino |
| | | acid |
| | | sequence |
| 33. | gaggtgcagc tggtggagtc tgggggaggt gtggtacggc ctggggggtc cctgagactc | DNA |
| | tcctgtacag cctctggatt cacctttgat gattatggca tgagctgggt ccgccaagct | nucleotide |
| | ccagggaagg gactggagtg ggtctctggt attaattgga atggtggtag aacagaatat | sequence |
| | gcagacgctg tgaagggccg attcatcatc tccagagaca acgccaagaa ctccctgtat | |
| | ctgcaaatga acagtctgag agccgaggac acggccttgt atttctgtgc gagaacggaa | |
| | ttacggtggg actactgggg ccagggaacc ctggtcaccg tctcctca | |
| 34. | EVQLVESGGG VVRPGGSLRL SCTASGFTFD DYGMSWVRQA PGKGLEWVSG INWNGGRTEY | AA amino |
| | ADAVKGRFII SRDNAKNSLY LQMNSLRAED TALYFCARTE LRWDYWGQGT LVTVSS | acid |
| | | sequence |
| 35. | ggattcacct ttgatgatta tggc | DNA |
| | | nucleotide |
| | | sequence |
| 36. | GFTFDDYG | AA amino |
| | | acid |
| | | sequence |
| 37. | attaattgga atggtggtag aaca | DNA |
| | | nucleotide |
| | | sequence |
| 38. | INWNGGRT | AA amino |
| | | acid |
| | | sequence |
| 39. | gcgagaacgg aattacggtg ggactac | DNA |
| | | nucleotide |
| | | sequence |
| 40. | ARTELRWDY | AA amino |
| | | acid |
| | | sequence |
| 41. | gaggtgcagc tggtggagtc tgggggaggc ttggtacagc ctggagggtc cctgagactc | DNA |
| | tcctgtgcag cctctggact caccttcagt agttatgaaa tgaactgggt ccgccaggct | nucleotide |
| | ccagggaagg ggctggagtg ggtttcacac attagtagtg ctggtagtac caaatactac | sequence |
| | gcagactctg tgaagggccg attcaccatc tccagagaca acgccaagaa ctcactgttt | |
| | ctgcaaatga acagcctgag agccgaggac acggctattt attactgtgc gggctataac | |
| | tacgcctact actacttcgg tgtggacgtc tggggccaag ggaccacggt caccgtctcc | |
| | tca | |
| 42. | EVQLVESGGG LVQPGGSLRL SCAASGLTFS SYEMNWVRQA PGKGLEWVSH ISSAGSTKYY | AA amino |
| | ADSVKGRFTI SRDNAKNSLF LQMNSLRAED TAIYYCAGYN YAYYYFGVDV WGQGTTVTVS | acid |
| | S | sequence |
| 43. | ggactcacct tcagtagtta tgaa | DNA |
| | | nucleotide |
| | | sequence |
| 44. | GLTFSSYE | AA amino |
| | | acid |
| | | sequence |
| 45. | attagtagtg ctggtagtac caaa | DNA |
| | | nucleotide |
| | | sequence |
| 46. | ISSAGSTK | AA amino |
| | | acid |
| | | sequence |
| 47. | gcgggctata actacgccta ctactacttc ggtgtggacg tc | DNA |
| | | nucleotide |
| | | sequence |
| 48. | AGYNYAYYYF GVDV | AA amino |
| | | acid |
| | | sequence |
| 49. | cagctgcagc tgcaggagtc gggcccagga ctggtgaagc cttcggagac cctgtccctc | DNA |
| | atttgcactg tctctggtgg ctccatcagc agtaataatt actactgggg ctggatccgc | nucleotide |
| | cagcccccag ggaaggggct ggaatggatt gggaatttgt atggtagtgg gagtacctac | sequence |
| | tacaacccgt ccctcaagag tcgagtcacc atatccgtag acacgtccaa gaaccagttc | |
| | tccctgaagc tgagttttgt gaccgccgca gacacggctg tgtatcactg tgcgagatgg | |
| | gacactaacg ggggggcttt tgatatctgg ggccaaggga caatggtcac cgtctcttca | |
| 50. | QLQLQESGPG LVKPSETLSL ICTVSGGSIS SNNYYWGWIR QPPGKGLEWI GNLYGSGSTY | AA amino |
| | YNPSLKSRVT ISVDTSKNQF SLKLSFVTAA DTAVYHCARW DTNGGAFDIW GQGTMVTVSS | acid |
| | | sequence |
| 51. | ggtggctcca tcagcagtaa taattactac | DNA |
| | | nucleotide |
| | | sequence |
| 52. | GGSISSNNYY | AA amino |
| | | acid |
| | | sequence |
| 53. | ttgtatggta gtgggagtac c | DNA |
| | | nucleotide |
| | | sequence |
| 54. | LYGSGST | AA amino |
| | | acid |
| | | sequence |
| 55. | gcgagatggg acactaacgg gggggctttt gatatc | DNA |
| | | nucleotide |
| | | sequence |
| 56. | ARWDTNGGAF DI | AA amino |
| | | acid |
| | | sequence |
| 57. | caggttcagc tggtgcagtc tggagctgag gtgaagaagc ctggggcctc agtgaaggtc | DNA |
| | tcctgcaagg cttctggtta cacctttacc agccttggaa tcagctgggt gcgacaggcc | nucleotide |
| | cctggacaag ggcttgagtg gatgggatgg atcagcgcct acagtggtaa tagagactat | sequence |
| | gcacagaagt tccagggcag agtcaccatg accacagaca catccacgag cacagcctat | |
| | atggagctga ggagcctgag atctgacgac acggccgtgt attattgtgc gagatctcta | |
| | cgttttgact actggggcca gggaaccctg gtcaccgtct cctca | |
| 58. | QVQLVQSGAE VKKPGASVKV SCKASGYTFT SLGISWVRQA PGQGLEWMGW ISAYSGNRDY | AA amino |
| | AQKFQGRVTM TTDTSTSTAY MELRSLRSDD TAVYYCARSL RFDYWGQGTL VTVSS | acid |
| | | sequence |
| 59. | ggttacacct ttaccagcct tgga | DNA |
| | | nucleotide |
| | | sequence |
| 60. | GYTFTSLG | AA amino |
| | | acid |
| | | sequence |
| 61. | atcagcgcct acagtggtaa taga | DNA |
| | | nucleotide |
| | | sequence |
| 62. | ISAYSGNR | AA amino |
| | | acid |
| | | sequence |
| 63. | gcgagatctc tacgttttga ctac | DNA |
| | | nucleotide |
| | | sequence |
| 64. | ARSLRFDY | AA amino |
| | | acid |
| | | sequence |
| 65. | gaggtgcagc tggtggagtc tgggggaggc ttggtacagc ctggcaggtc cctgagactc | DNA |
| | tcctgtgcag cctcaggatt caccttggat gattttgcca tgcactgggt ccggcaagct | nucleotide |
| | ccagggaagg gcctggagtg ggtctcaggt attagttgga atagcggtaa tataggttat | sequence |
| | gcggactctg tgaagggccg cttcaccatc tccagagaca acgccaggaa ctccctgtat | |
| | ctgcaaatga acagtctgag agcggaagac acggccttgt attactgcac ctcgggatac | |
| | tacctctact acgctatgga cgtctggggc caggggacca cggtcaccgt ctcctca | |
| 66. | EVQLVESGGG LVQPGRSLRL SCAASGFTLD DFAMHWVRQA PGKGLEWVSG ISWNSGNIGY | AA amino |
| | ADSVKGRFTI SRDNARNSLY LQMNSLRAED TALYYCTSGY YLYYAMDVWG QGTTVTVSS | acid |
| | | sequence |
| 67. | ggattcacct tggatgattt tgcc | DNA |
| | | nucleotide |
| | | sequence |
| 68. | GFTLDDFA | AA amino |
| | | acid |
| | | sequence |
| 69. | attagttgga atagcggtaa tata | DNA |
| | | nucleotide |
| | | sequence |
| 70. | ISWNSGNI | AA amino |
| | | acid |
| | | sequence |
| 71. | acctcgggat actacctcta ctacgctatg gacgtc | DNA |
| | | nucleotide |
| | | sequence |
| 72. | TSGYYLYYAM DV | AA amino |
| | | acid |
| | | sequence |
| 73. | caggtgcagc tggtgcagtc tgggcctgag gtgaagaagc ctgggtcttc ggtgaaggtc | DNA |
| | tcctgcaagg cttctggagg caccatcgac agaaatgaaa tcacctgggt gcgacaggcc | nucleotide |
| | cctggacaag ggcttgagtg gatgggagta atcatcccta tcttaggtga ggcacactac | sequence |
| | gaacgaaaat tccagggcag actcacgatt accatggacg agtccacgag tacagcctat | |
| | atggaactga gtaatctgag atctgaggac acggccgtat attactgtgc gagagagagg | |
| | gcagctcgag gctaccttga ctactggggc cagggaaccc tggtcaccgt ctcctca | |
| 74. | QVQLVQSGPE VKKPGSSVKV SCKASGGTID RNEITWVRQA PGQGLEWMGV IIPILGEAHY | AA amino |
| | ERKFQGRLTI TMDESTSTAY MELSNLRSED TAVYYCARER AARGYLDYWG QGTLVTVSS | acid |
| | | sequence |
| 75. | ggaggcacca tcgacagaaa tgaa | DNA |
| | | nucleotide |
| | | sequence |
| 76. | GGTIDRNE | AA amino |
| | | acid |
| | | sequence |
| 77. | atcatcccta tcttaggtga ggca | DNA |
| | | nucleotide |
| | | sequence |
| 78. | IIPILGEA | AA amino |
| | | acid |
| | | sequence |
| 79. | gcgagagaga gggcagctcg aggctacctt gactac | DNA |
| | | nucleotide |
| | | sequence |
| 80. | ARERAARGYL DY | AA amino |
| | | acid |
| | | sequence |
| 81. | gaggtgcagc tggtggagtc tgggggaggc ttggtccagc cgggggggtc cctgagactc | DNA |
| | tcctgtacag cctctggatt caccttcagt aattatgcta tattctgggt ccgccaggct | nucleotide |
| | ccagggaagg gactggaata tgtttcaggt attagtaata atggggttac taaatattat | sequence |
| | gcagactctg tgagggacag attcaccatc tccagagaca attccaggaa cacggtgtct | |
| | attcaaatgg acagcctgag aactgaggac atggctgtgt attactgtgc gcgagaaaac | |
| | tggggatcgg acgatggttt tgatatctgg ggccaaggaa caatggtcac cgtctcttca | |
| 82. | EVQLVESGGG LVQPGGSLRL SCTASGFTFS NYAIFWVRQA PGKGLEYVSG ISNNGVTKYY | AA amino |
| | ADSVRDRFTI SRDNSRNTVS IQMDSLRTED MAVYYCAREN WGSDDGFDIW GQGTMVTVSS | acid |
| | | sequence |
| 83. | ggattcacct tcagtaatta tgct | DNA |
| | | nucleotide |
| | | sequence |
| 84. | GFTFSNYA | AA amino |
| | | acid |
| | | sequence |
| 85. | attagtaata atggggttac taaa | DNA |
| | | nucleotide |
| | | sequence |
| 86. | ISNNGVTK | AA amino |
| | | acid |
| | | sequence |
| 87. | gcgcgagaaa actggggatc ggacgatggt tttgatatc | DNA |
| | | nucleotide |
| | | sequence |
| 88. | ARENWGSDDG FDI | AA amino |
| | | acid |
| | | sequence |
| 89. | caggtgcagt tggtgcagtc tgggactgag gtgaagaagc ctgggtcctc ggtgaaggtc | DNA |
| | tcctgcaagg cttctggagg caccttcagc acctatccta tcaattgggt gcgacaggcc | nucleotide |
| | cctggacaag ggcttgagtg gatgggaggg atcatcccta tctttggtac ggcaagctac | sequence |
| | gcacagaagt tccagggcag agtcgcgatt accacggacg aatccacgag cacagcctac | |
| | atggagctga acagcctgag atctgaggac acggccgtgt attattgtgc gagagagagg | |
| | ccgaactggg gatctaatga ctactggggc cagggaaccc tggtcaccgt ctcctca | |
| 90. | QVQLVQSGTE VKKPGSSVKV SCKASGGTFS TYPINWVRQA PGQGLEWMGG IIPIFGTASY | AA amino |
| | AQKFQGRVAI TTDESTSTAY MELNSLRSED TAVYYCARER PNWGSNDYWG QGTLVTVSS | acid |
| | | sequence |
| 91. | ggaggcacct tcagcaccta tcct | DNA |
| | | nucleotide |
| | | sequence |
| 92. | GGTFSTYP | AA amino |
| | | acid |
| | | sequence |
| 93. | atcatcccta tctttggtac ggca | DNA |
| | | nucleotide |
| | | sequence |
| 94. | IIPIFGTA | AA amino |
| | | acid |
| | | sequence |
| 95. | gcgagagaga ggccgaactg gggatctaat gactac | DNA |
| | | nucleotide |
| | | sequence |
| 96. | ARERPNWGSN DY | AA amino |
| | | acid |
| | | sequence |
| 97. | caggtgcagc tggtggagtc tgggggaggc gtggtccagc ctgggaggtc cctgagactc | DNA |
| | gcctgtgcag cgtctggatt cacctttaga agttatggca tgcactgggt ccgccaggct | nucleotide |
| | ccaggcaagg ggctggagtg ggtggcagtt atctggtatg atggaagtaa taaatactat | sequence |
| | acagactccg tgaagggccg attcaccatc tccagagaca attccaagaa cacgctgtat | |
| | ctgcaaatga acagcctgag agccgaggac acggctatgt attactgtgc gatagacaaa | |
| | tgggctacgg tgactacgta cctgtttgac tactggggcc agggaaccct ggtcaccgtc | |
| | tcctca | |
| 98. | QVQLVESGGG VVQPGRSLRL ACAASGFTFR SYGMHWVRQA PGKGLEWVAV IWYDGSNKYY | AA amino |
| | TDSVKGRFTI SRDNSKNTLY LQMNSLRAED TAMYYCAIDK WATVTTYLFD YWGQGTLVTV | acid |
| | SS | sequence |
| 99. | ggattcacct ttagaagtta tggc | DNA |
| | | nucleotide |
| | | sequence |
| 100. | GFTFRSYG | AA amino |
| | | acid |
| | | sequence |
| 101. | atctggtatg atggaagtaa taaa | DNA |
| | | nucleotide |
| | | sequence |
| 102. | IWYDGSNK | AA amino |
| | | acid |
| | | sequence |
| 103. | gcgatagaca aatgggctac ggtgactacg tacctgtttg actac | DNA |
| | | nucleotide |
| | | sequence |
| 104. | AIDKWATVTT YLFDY | AA amino |
| | | acid |
| | | sequence |
| 105. | gaggtgcagc tggtggagtc tgggggaggc ttggtacagt ctggagggtc cctgagactc | DNA |
| | tcttgtgcag cctctggatt caccttcagt cgttatgaaa tgaactgggt ccgccaggct | nucleotide |
| | ccagggaagg ggctggagtg ggttgcatat attagtaata ctggtactac cagggactat | sequence |
| | tcagactctg tgaagggccg attcaccatc tccagagaca acgccaagaa ctcactatat | |
| | ctacaaatga gcagcctgag agtcgaggac acggctgttt attattgtgt gagagagaaa | |
| | tttagtggga cctctgactg gttcgacccc tggggccagg gaaccctggt caccgtctcc | |
| | tca | |
| 106. | EVQLVESGGG LVQSGGSLRL SCAASGFTFS RYEMNWVRQA PGKGLEWVAY ISNTGTTRDY | AA amino |
| | SDSVKGRFTI SRDNAKNSLY LQMSSLRVED TAVYYCVREK FSGTSDWFDP WGQGTLVTVS | acid |
| | S | sequence |
| 107. | ggattcacct tcagtgtta tgaa | |
| | | DNA |
| | | nucleotide |
| | | sequence |
| 108. | GFTFSRYE | AA amino |
| | | acid |
| | | sequence |
| 109. | attagtaata ctggtactac cagg | DNA |
| | | nucleotide |
| | | sequence |
| 110. | ISNTGTTR | AA amino |
| | | acid |
| | | sequence |
| 111. | gtgagagaga aatttagtgg gacctctgac tggttcgacc cc | DNA |
| | | nucleotide |
| | | sequence |
| 112. | VREKFSGTSD WFDP | AA amino |
| | | acid |
| | | sequence |
| 113. | gaggtgcagc tggtggagtc tgggggaggc ttggtgcagc ctggggggtc cctgagactc | DNA |
| | tcctgtgcag cctctggatt cacctttagt agctatgcca tgaactgggt ccgccaggct | nucleotide |
| | ccagggaagg ggctggaatg ggtctcagct attagtggta gtggtgatag cacatactac | sequence |
| | acagactccg tgaagggccg gttcaccatc tccagagaca attccaagaa cacgctgtat | |
| | ctgcaaatga acaccctgag agccgaggac acggccgtat attactgtgc gaaagaggag | |
| | attgttatgt tcgtaggtgc taccccgggg tggttcgacc cctggggcca gggaaccctg | |
| | gtcaccgtct cctca | |
| 114. | EVQLVESGGG LVQPGGSLRL SCAASGFTFS SYAMNWVRQA PGKGLEWVSA ISGSGDSTYY | AA amino |
| | TDSVKGRFTI SRDNSKNTLY LQMNTLRAED TAVYYCAKEE IVMLVGATPG WFDPWGQGTL | acid |
| | VTVSS | sequence |
| 115. | ggattcacct ttagtagcta tgcc | DNA |
| | | nucleotide |
| | | sequence |
| 116. | GFTFSSYA | AA amino |
| | | acid |
| | | sequence |
| 117. | attagtggta gtggtgatag caca | DNA |
| | | nucleotide |
| | | sequence |
| 118. | ISGSGDST | AA amino |
| | | acid |
| | | sequence |
| 119. | gcgaaagagg agattgttat gttggtaggt gctaccccgg ggtggttcga cccc | DNA |
| | | nucleotide |
| | | sequence |
| 120. | AKEEIVMLVG ATPGWFDP | AA amino |
| | | acid |
| | | sequence |
| 121. | gaggtgcagc tggtgcagtc tggagcagag gtgaaaaagc ccggggagtc tctgaagatc | DNA |
| | tcctgtaagg gttctggata cagttttacc agttactgga tcgactgggt gcgccagatg | nucleotide |
| | cccgggaaag gcctggagtg gatggggatc atctatcctg atgactctga taccagatac | sequence |
| | agtccgtcct tccaaggcca ggtcaccatg tcagtcgaca agtccatcag caccgcctac | |
| | ctgcagtgga gcagcctgaa ggcctcggac accgccatat attactgtgc gagacatgag | |
| | agaggtatag tagaagctgg tacggattac tactactact acggtatgga cgtctggggc | |
| | caagggacca cggtcaccgt ctcctca | |
| 122. | EVQLVQSGAE VKKPGESLKI SCKGSGYSFT SYWIDWVRQM PGKGLEWMGI IYPDDSDTRY | AA amino |
| | SPSFQGQVTM SVDKSISTAY LQWSSLKASD TAIYYCARHE RGIVEAGTDY YYYYGMDVWG | acid |
| | QGTTVTVSS | sequence |
| 123. | ggatacagtt ttaccagtta ctgg | DNA |
| | | nucleotide |
| | | sequence |
| 124. | GYSFTSYW | AA amino |
| | | acid |
| | | sequence |
| 125. | atctatcctg atgactctga tacc | DNA |
| | | nucleotide |
| | | sequence |
| 126. | IYPDDSDT | AA amino |
| | | acid |
| | | sequence |
| 127. | gcgagacatg agagaggtat agtagaagct ggtacggatt actactacta ctacggtatg | DNA |
| | gacgtc | nucleotide |
| | | sequence |
| 128. | ARHERGIVEA GTDYYYYYGM DV | AA amino |
| | | acid |
| | | sequence |
| 129. | caggttcagc tggtgcagtc tggagctgag gtgatgaagc ctggggcctc agtgaaggtc | DNA |
| | tcctgcaggg cttctggtta cacctttagc agctatgata tcaactgggt gcgacaggcc | nucleotide |
| | cctggacaag gacttgagtg gatgggatgg atcagcactg ccactggtaa cacaaactat | sequence |
| | ccacagaagg tccagggcag agtcaccatg accacagaca catcaacgaa cacagcctac | |
| | atggagctga ggagcctgag atctgacgac acggccgtat attattgtac gagagagggc | |
| | aattcgaagg gctggctcga cccctggggc cagggaaccc tggtcaccgt ctcctca | |
| 130. | QVQLVQSGAE VMKPGASVKV SCRASGYTFS SYDINWVRQA PGQGLEWMGW ISTATGNTNY | AA amino |
| | PQKVQGRVTM TTDTSTNTAY MELRSLRSDD TAVYYCTREG NSKGWLDPWG QGTLVTVSS | acid |
| | | sequence |
| 131. | ggttacacct ttagcagcta tgat | DNA |
| | | nucleotide |
| | | sequence |
| 132. | GYTFSSYD | AA amino |
| | | acid |
| | | sequence |
| 133. | atcagcactg ccactggtaa caca | DNA |
| | | nucleotide |
| | | sequence |
| 134. | ISTATGNT | AA amino |
| | | acid |
| | | sequence |
| 135. | acgagagagg gcaattcgaa gggctggctc gacccc | DNA |
| | | nucleotide |
| | | sequence |
| 136. | TREGNSKGWL DP | AA amino |
| | | acid |
| | | sequence |
| 137. | caggtacagc tgcagcagtc aggtcccgga caaatgaagt cctcgcagac cctcacactc | DNA |
| | acctgtgcca tctccgggga cagtgtctct agcaacagtg ctatttggta ctggatcagg | nucleotide |
| | cagtccccat cgagaggcct tgagtggctg gggaggacaa agttcaggtc caaatggtat | sequence |
| | aatgattttg cactatctat gaaaagtcga ataaccatca acccagacac atccaagaac | |
| | cagttctccc tgcacctgaa ctctgtgact cccgaggaca cggctgtata ttactgtgca | |
| | agatataagt gggaacttta ctttgactac tggggccagg gaaccctggt caccgtctcc | |
| | tca | |
| 138. | QVQLQQSGPG QMKSSQTLTL TCAISGDSVS SNSAIWYWIR QSPSRGLEWL GRTKFRSKWY | AA amino |
| | NDFALSMKSR ITINPDTSKN QFSLHLNSVT PEDTAVYYCA RYKWELYFDY WGQGTLVTVS | acid |
| | S | sequence |
| 139. | ggggacagtg tctctagcaa cagtgctatt | DNA |
| | | nucleotide |
| | | sequence |
| 140. | GDSVSSNSAI | AA amino |
| | | acid |
| | | sequence |
| 141. | acaaagttca ggtccaaatg gtataat | DNA |
| | | nucleotide |
| | | sequence |
| 142. | TKFRSKWYN | AA amino |
| | | acid |
| | | sequence |
| 143. | gcaagatata agtgggaact ttactttac tac | DNA |
| | | nucleotide |
| | | sequence |
| 144. | ARYKWELYFD Y | AA amino |
| | | acid |
| | | sequence |
| 145. | gaggtgcagc tggtggagtc tgggggaggc ttggtccagc ctggggggtc cctgagactc | DNA |
| | tcctgtgcag cctctggatt cacctttagt acctattgga tgaactgggt ccgccaggct | nucleotide |
| | ccagggaagg ggctggagtg ggtggccaac ataaaggaag atggaagtga gaaatactat | sequence |
| | gtggactctg tgaagggccg attcaccatc tccagagaca acgccaagaa ctcactgtat | |
| | ctgcaaatga acagcctgag agccgaggac acggctgtgt attactgtgc gagagagggt | |
| | tttggagtgg ttacctatat ctggggccaa gggacaatgg tcaccgtctc ttca | |
| 146. | EVQLVESGGG LVQPGGSLRL SCAASGFTFS TYWMNWVRQA PGKGLEWVAN IKEDGSEKYY | AA amino |
| | VDSVKGRFTI SRDNAKNSLY LQMNSLRAED TAVYYCAREG FGVVTYIWGQ GTMVTVSS | acid |
| | | sequence |
| 147. | ggattcacct ttagtaccta ttgg | DNA |
| | | nucleotide |
| | | sequence |
| 148. | GFTFSTYW | AA amino |
| | | acid |
| | | sequence |
| 149. | ataaaggaag atggaagtga gaaa | DNA |
| | | nucleotide |
| | | sequence |
| 150. | IKEDGSEK | AA amino |
| | | acid |
| | | sequence |
| 151. | gcgagagagg gttttggagt ggttacctat atc | DNA |
| | | nucleotide |
| | | sequence |
| 152. | AREGFGVVTY I | AA amino |
| | | acid |
| | | sequence |
| 153. | caggtgcagc tggtggagtc tgggggaggc ttggtcaagc ctggagggtc cctgagactc | DNA |
| | tcctgtgcag cctctggatt caccttcagt gactactata tgacctggat ccgccagact | nucleotide |
| | ccagggaagg ggctggattg ggtttcatac attagttcta gtggtactaa caaatacaac | sequence |
| | gcagactctg tgaagggccg attcaccatc tccagggaca acgccaagaa ctcactgtat | |
| | ctgcaaatga acagcctgcg agccgaggac acggccgtat attactgtgt gagagacccc | |
| | ccctggggta tggacgtctg gggccaaggg accacggtca ccgtctcctc a | |
| 154. | QVQLVESGGG LVKPGGSLRL SCAASGFTFS DYYMTWIRQT PGKGLDWVSY ISSSGTNKYN | AA amino |
| | ADSVKGRFTI SRDNAKNSLY LQMNSLRAED TAVYYCVRDP PWGMDVWGQG TTVTVSS | acid |
| | | sequence |
| 155. | ggattcacct tcagtgacta ctat | DNA |
| | | nucleotide |
| | | sequence |
| 156. | GFTFSDYY | AA amino |
| | | acid |
| | | sequence |
| 157. | attagttcta gtggtactaa caaa | DNA |
| | | nucleotide |
| | | sequence |
| 158. | ISSSGTNK | AA amino |
| | | acid |
| | | sequence |
| 159. | gtgagagacc ccccctgggg tatggacgtc | DNA |
| | | nucleotide |
| | | sequence |
| 160. | VRDPPWGMDV | AA amino |
| | | acid |
| | | sequence |
| 161. | gaggtgcagc tggtggagtc tgggggaggc ttggtacagc ctggggggtc cctgagactc | DNA |
| | tcctgtgttg cctctggatt caccttcaat aaatacgaca tgcactgggt ccgccaaact | nucleotide |
| | actggaaaag gtctagagtg ggtctcaggt attgatactg atggtgacac atactatcca | sequence |
| | ggctccgtga agggccgatt caccatctcc agagaaaatg ccgagaactc cctgtatctt | |
| | caaatgaacg gcctgagagt cggggacacg gctgtgtatt actgtgcaag atggccttgg | |
| | agtggtttct atggtgcttt tgatatctgg ggccaaggga caatggtcac cgtctcttca | |
| 162. | EVQLVESGGG LVQPGGSLRL SCVASGFTFN KYDMHWVRQT TCKGLEWVSG IDTDGDTYYP | AA |
| | GSVKGRFTIS RENAENSLYL QMNGLRVGDT AVYYCARWPW SGFYGAFDIW GQGTMVTVSS | acid |
| | | sequence |
| 163. | ggattcacct tcaataaata cgac | DNA |
| | | nucleotide |
| | | sequence |
| 164. | GFTFNKYD | AA amino |
| | | acid |
| | | sequence |
| 165. | attgatactg atggtgacac a | DNA |
| | | nucleotide |
| | | sequence |
| 166. | IDTDGDT | AA amino |
| | | acid |
| | | sequence |
| 167. | gcaagatggc cttggagtgg tttctatggt gcttttgata tc | DNA |
| | | nucleotide |
| | | sequence |
| 168. | ARWPWSGFYG AFDI | AA amino |
| | | acid |
| | | sequence |
| 169. | caggtgcagc tgcaggagtc gggcccagga ctggtgaagc cttcacagac cctgtccctc | DNA |
| | acctgcactg tctctggtgg ctccatcagc agcggtggtg actactggag ctggatccgc | nucleotide |
| | cagctcccag ggaagggcct ggagtggatt gggtacatct attacagtgg gagcgcctac | sequence |
| | tataatccgt ccctcaagag tcgaggtacc atatcaatag acacgtctaa gaaccagttc | |
| | tccctgaagc tgacctctgt gactgccgcg gacacggccg tatatttctg tgtgaaatta | |
| | cgatttttgg agtggttctt ggggggctgg ttcggcccct ggggccaggg aaccctggtc | |
| | accgtctcct ca | |
| 170. | QVQLQESGPG LVKPSQTLSL TCTVSGGSIS SGGDYWSWIR QLPGKGLEWI GYIYYSGSAY | AA amino |
| | YNPSLKSRGT ISIDTSKNQF SLKLTSVTAA DTAVYFCVKL RFLEWFLGGW FGPWGQGTLV | acid |
| | TVSS | sequence |
| 171. | ggtggctcca tcagcagcgg tggtgactac | DNA |
| | | nucleotide |
| | | sequence |
| 172. | GGSISSGGDY | AA amino |
| | | acid |
| | | sequence |
| 173. | atctattaca gtgggagcgc c | DNA |
| | | nucleotide |
| | | sequence |
| 174. | IYYSGSA | AA amino |
| | | acid |
| | | sequence |
| 175. | gtgaaattac gatttttgga gtggttcttg gggggctggt tcggcccc | DNA |
| | | nucleotide |
| | | sequence |
| 176. | VKLRFLEWFL GGWFGP | AA amino |
| | | acid |
| | | sequence |
| 177. | gaggtgcagc tggtggagtc tgggggaggc ttggtacagc cgggggggtc cctgagactc | DNA |
| | tcctgtgcag cctctggatt cacctttagc aactatggca tgacctgggt ccgccaggct | nucleotide |
| | ccagggaagg gcctggaatg ggtctcagct attactggtg gtggtggtag cacatactac | sequence |
| | tcaaactccg tgaagggccg gttcaccatc tccagagaca attccaagaa cacggtgtat | |
| | ctgcgaatga acagtgtgag agccgaggac acggccgtat attactgtgc gaaatataag | |
| | tggaacttcg tggacgactg gggccaggga accacggtca ccgtctcctc a | |
| 178. | EVQLVESGGG LVQPGGSLRL SCAASGFTFS NYGMTWVRQA PGKGLEWVSA ITGGGGSTYY | AA amino |
| | SNSVKGRFTI SRDNSKNTVY LRMNSVRAED TAVYYCAKYK WNFVDDWGQG TTVTVSS | acid |
| | | sequence |
| 179. | ggattcacct ttagcaacta tggc | DNA |
| | | nucleotide |
| | | sequence |
| 180. | GFTFSNYG | AA amino |
| | | acid |
| | | sequence |
| 181. | attactggtg gtggtggtag caca | DNA |
| | | nucleotide |
| | | sequence |
| 182. | ITGGGGST | AA amino |
| | | acid |
| | | sequence |
| 183. | gcgaaatata agtggaactt cgtggacgac | DNA |
| | | nucleotide |
| | | sequence |
| 184. | AKYKWNFVDD | AA amino |
| | | acid |
| | | sequence |
| 185. | MLTLQTWLVQ ALFIFLTTES TGELLDPCGY ISPESPVVQL HSNFTAVCVL KEKCMDYFHV | AA amino |
| | NANYIVWKTN HFTIPKEQYT IINRTASSVT FTDIASLNIQ LTCNILTFGQ LEQNVYGITI | acid |
| | ISGLPPEKPK NLSCIVNEGK KMRCEWDGGR ETHLETNFTL KSEWATHKFA DCKAKRDTPT | sequence |
| | SCTVDYSTVY FVNIEVWVEA ENALGKVTSD HINFDPVYKV KPNPPHNLSV INSEELSSIL | |
| | KLTWTNPSIK SVIILKYNIQ YRTKDASTWS QIPPEDTAST RSSFTVQDLK PFTEYVFRIR | |
| | CMKEDGKGYW SDWSEEASGI TYEDRPSKAP SFWYKIDPSH TQGYRTVQLV WKTLPPFEAN | |
| | GKILDYEVTL TRWKSHLQNY TVNATKLTVN LTNDRYLATL TVRNLVGKSD AAVLTIPACD | |
| | FQATHPVMDL KAFPKDNMLW VEWTTPRESV KKYILEWCVL SDKAPCITDW QQEDGTVHRT | |
| | YLRGNLAESK CYLITVIPVY ADGPGSPESI KAYLKQAPPS KGPTVRTKKV GKNEAVLEWD | |
| | QLPVDVQNGF IRNYTIFYRT IIGNETAVNV DSSHTEYTLS SLTSDTLYMV RMAAYTDEGG | |
| | KDGPEFTFTT PKFAQGEIEA IVVPVCLAFL LTILLGVLFC FNKRDLIKKH IWPNVPDPSK | |
| | SHIAQWSPHT PPRHNFNSKD QMYSDGNFTD VSVVEIEAND KKPFPEDLKS LDLFKKEKIN | |
| | TEGHSSGIGG SSCMSSSRPS ISSSDENESS QNTSSTVQYS TVVHSGYRHQ VPSVQVFSRS | |
| | ESTQPLLDSE ERPEDLQLVD HVDGGDGILP RQQYFKQNCS QHESSPDISH FERSKQVSSV | |
| | NEEDFVRLKQ QISDHISQSC GSGQMKMFQE VSAADAFGPG TEGQVERFET VGMEAATDEG | |
| | MPKSYLPQTV RQGGYMPQ | |
| 186. | MICQKFCVVL LHWEFIYVIT AFNLSYPITP WRFKLSCMPP NSTYDYFLLP AGLSKNTSNS | AA amino |
| | NGHYETAVEP KFNSSGTHFS NLSKTTFHCC FRSEQDRNCS LCADNIEGKT FVSTVNSLVF | acid |
| | QQIDANWNIQ CWLKGDLKLF ICYVESLFKN LFRNYNYKVH LLYVLPEVLE DSPLVPQKGS | sequence |
| | FQMVHCNCSV HECCECLVPV PTAKLNDTLL MCLKITSGGV IFQSPLMSVQ PINMVKPDPP | |
| | LGLHMEITDD GNLKISWSSP PLVPFPLQYQ VKYSENSITV IREADKIVSA TSLLVDSILP | |
| | GSSYEVQVRG KRLDGPGIWS DWSTPRVFTT QDVIYFPPKI LTSVGSNVSF HCIYKKENKI | |
| | VPSKEIVWWM NLAEKIPQSQ YDVVSDHVSK VTFFNLNETK PRGKFTYDAV YCCNEHECHH | |
| | RYAELYVIDV NINISCETDG YLTKMTCRWS TSTIQSLAES TLQLRYHRSS LYCSDIPSIH | |
| | PISEPKDCYL QSDGFYECIF QPIFLLSGYT MWIRINHSLG SLDSPPTCVL PDSVVKPLPP | |
| | SSVKAEITIN IGLLKISWEK PVFPENNLQF QIRYGLSGKE VQWKMYEVYD AKSKSVSLPV | |
| | PDLCAVYAVQ VRCKRLDGLG YWSNWSNPAY TVVMDIKVPM RGPEFWRIIN GDTMKKEKNV | |
| | TLLWKPLMKN DSLCSVQRYV INHHTSCNGT WSEDVGNHTK FTFLWTEQAH TVIVLAINSI | |
| | GASVANFNLT FSWPMSKVNI VQSLSAYPLN SSCVIVSWIL SPSDYKLMYF IIEWKNLNED | |
| | GEIKWLRISS SVKKYYIHDH FIPIEKYQFS LYPIFMEGVG KPKIINSFTQ DDIEKHQSDA | |
| | GLYVIVPVII SSSILLLGTL LISHQRMKKL FWEDVPNPKN CSWAQGLNFQ KPETFEHLFI | |
| | KHTASVTCGP LLLEPETISE DISVDISWKN KDEMMPTTVV SLLSTIDLEK GSVCISDQFN | |
| | SVNFSEAEGT EVTYEDESQR QPFVKYATLI SNSKPSETGE EQGLINSSVT KCFSSKNSPL | |
| | KDSFSNSSWE IEAQAFFILS DQHPNIISPH LTFSEGLDEL LKLEGNFPEE NNDKKSIYYL | |
| | GVTSIKKRES GVLLTDKSRV SCPFPAPCLF TDIRVLQDSC SHFVENNINL GTSSKKTFAS | |
| | YMPQFQTCST QTHKIMENKM CDLTV | |
| 187. | FNLSYPITPW RFKLSCMPPN STYDYFLLPA GLSKNTSNSN GHYETAVEPK FNSSGTHFSN | AA amino |
| | LSKTTFHCCF RSEQDRNCSL CADNIEGKTF VSTVNSLVFQ QIDANWNIQC WLKGDLKLFI | acid |
| | CYVESLFKNL FRNYNYKVHL LYVLPEVLED SPLVPQKGSF QMVHCNCSVH ECCECLVPVP | sequence |
| | TAKLNDTLLM CLKITSGGVI FQSPLMSVQP INMVKPDPPL GLHMEITDDG NLKISWSSPP | |
| | LVPFPLQYQV KYSENSTTVI READKIVSAT SLLVDSILPG SSYEVQVRGK RLDGPGIWSD | |
| | WSTPRVFTTQ DVIYFPPKIL TSVGSNVSFH CIYKKENKIV PSKEIVWWMN LAEKIPQSQY | |
| | DVVSDHVSKV TFFNLNETKP RGKFTYDAVY CCNEHECHHR YAELYVIDVN INISCETDGY | |
| | LTKMTCRWST STIQSLAEST LQLRYHRSSL YCSDIPSIHP ISEPKDCYLQ SDGFYECIFQ | |
| | PIFLLSGYTM WIRINHSLGS LDSPPTCVLP DSVVKPLPPS SVKAEITINI GLLKISWEKP | |
| | VFPENNLQFQ IRYGLSGKEV QWKMYEVYDA KSKSVSLPVP DLCAVYAVQV RCKRLDGLGY | |
| | WSNWSNPAYT VVMDIKVPMR GPEFWRIING DTMKKEKNVT LLWKPLMKND SLCSVQRYVI | |
| | NHHTSCNGTW SEDVGNHTKF TFLWTEQAHT VTVLAINSIG ASVANFNLTF SWPMSKVNIV | |
| | QSLSAYPLNS SCVIVSWILS PSDYKLMYFI IEWKNLNEDG EIKWLRISSS VKKYYIHDHF | |
| | IPIEKYQFSL YPIFMEGVGK PKIINSFTQD DIEKHQSDEQ KLISEEDLGG EQKLISEEDL | |
| | HHHHHH | |
| 188. | FNLSYPITPW RFKLSCMPPN STYDYFLLPA GLSKNTSNLN GHYETAVEFN SSDTHESNLS | AA amino |
| | KTTFHCCFRS EQDRNCSLCA DNIEGKTFVS TVNSSVFQQM GANWNIQCWL KGDLKLFICY | acid |
| | VESLFKNPFK NYKHKVHLLY VLPEVLEDSP LVPQKGSFQM VHCNCSVHER CECLVPVPTA | sequence |
| | KLNDTLLMCL KITSGGVIFQ SPLMSVQPIN MVKPDPPLGL RMEITDDGNL KISWSSPPLV | |
| | PFPLQYEVKY SENSTTVIRE ADKIVSATSL LVDGILPGSS YEVQVRGKRL DGPGIWSDWS | |
| | TPHVFTTQDV IYFPPKILTS VGSNVSFHCI YKNENKIVSS KKIVWWMNLA EKIPQSQYDV | |
| | VSDHVSKVTF FNLNETKPRG KFTYDAVYCC NEHECHHRYA ELYVIDVNIN ISCETDGHLT | |
| | KMTCRWSTNT IQSLAGSTLQ LRYRRSSLYC FDIPSIHPIS KPKDCYLQSD GFYECVFQPI | |
| | FLLSGYTMWI RINHPLGSLD SPPTCVLPDS VVKPLPPSSV KAEIIKNIGL LKISWEKPVF | |
| | PENNLQFQIR YGLSGKEIQW KMYDVYDAKS KSVSLPVPDF CAVYAVQVRC KRSDGLGLWS | |
| | NWSNPAYTVV MDIKVPMRGP EFWRIINGDT MKKEKNVTLL WKPLMKNDSL CSVQRYVINH | |
| | HTSCNGTWSE DVGNHIKFTF LWTEQAHTVT VLAINSIGAS VANFNLTFSW PMSKVNIVQS | |
| | LSAYPLNSSC VILSWILSPS DYKLMYFIIE WKNLNEDGEI KWLRISSSVK KYYIHDHFIP | |
| | IEKYQFSLYP IFMEGVGKPK IINSFAQDNT EKHQNDEQKL ISEEDLGGEQ KLISEEDLHH | |
| | HHHH | |
| 189. | FNLSYPITPW RFKLSCMPPN STYDYFLLPA GLSKNTSNSN GHYETAVEPK FNSSGTHFSN | AA amino |
| | LSKTTFHCCF RSEQDRNCSL CADNIEGKTF VSTVNSLVFQ QIDANWNIQC WLKGDLKLFI | acid |
| | CYVESLFKNL FRNYNYKVHL LYVLPEVLED SPLVPQKGSF QMVHCNCSVH ECCECLVPVP | sequence |
| | TAKLNDTLLM CLKITSGGVI FQSPLMSVQP INMVKPDPPL GLHMEITDDG NLKISWSSPP | |
| | LVPFPLQYQV KYSENSTTVI READKIVSAT SLLVDSILPG SSYEVQVRGK RLDGPGIWSD | |
| | WSTPRVFTTQ DVIYFPPKIL TSVGSNVSFH CIYKKENKIV PSKEIVWWMN LAEKIPQSQY | |
| | DVVSDHVSKV TFFNLNETKP RGKFTYDAVY CCNEHECHHR YAELYVIDVN INISCETDGY | |
| | LTKMTCRWST STIQSLAEST LQLRYHRSSL YCSDIPSIHP ISEPKDCYLQ SDGFYECIFQ | |
| | PIFLLSGYTM WIRINHSLGS LDSPPTCVLP DSVVKPLPPS SVKAEITINI GLLKISWEKP | |
| | VFPENNLQFQ IRYGLSGKEV QWKMYEVYDA KSKSVSLPVP DLCAVYAVQV RCKRLDGLGY | |
| | WSNWSNPAYT VVMDIKVPMR GPEFWRIING DTMKKEKNVT LLWKPLMKND SLCSVQRYVI | |
| | NHHTSCNGTW SEDVGNHTKF TFLWTEQAHT VTVLAINSIG ASVANFNLTF SWPMSKVNIV | |
| | QSLSAYPLNS SCVIVSWILS PSDYKLMYFI IEWKNLNEDG EIKWLRISSS VKKYYIHDHF | |
| | IPIEKYQFSL YPIFMEGVGK PKIINSFTQD DIEKHQSDDK THTCPPCPAP ELLGGPSVFL | |
| | FPPKPKDTLM ISRTPEVTCV VVDVSHEDPE VKFNWYVDGV EVHNAKTKPR EEQYNSTYRV | |
| | VSVLTVLHQD WLNGKEYKCK VSNKALPAPI EKTISKAKGQ PREPQVYTLP PSRDELTKNQ | |
| | VSLTCLVKGF YPSDIAVEWE SNGQPENNYK TTPPVLDSDG SFFLYSKLTV DKSRWQQGNV | |
| | FSCSVMHEAL HNHYTQKSLS LSPGK | |
| 190. | ELLDPCGYIS PESPVVQLHS NFTAVCVLKE KCMDYFHVNA NYIVWKINHF TIPKEQYTII | AA amino |
| | NRTASSVTFT DIASLNIQLT CNILTFGQLE QNVYGITIIS GLPPEKPKNL SCIVNEGKKM | acid |
| | RCEWDGGRET HLETNFTLKS EWATHKFADC KAKRDTPTSC TVDYSTVYFV NIEVWVEAEN | sequence |
| | ALGKVTSDHI NFDPVYKVKP NPPHNLSVIN SEELSSILKL TWTNPSIKSV IILKYNIQYR | |
| | TKDASTWSQI PPEDTASTRS SFTVQDLKPF TEYVFRIRCM KEDGKGYWSD WSEEASGITY | |
| | EDRPSKAPSF WYKIDPSHTQ GYRTVQLVWK TLPPFEANGK ILDYEVTLTR WKSHLQNYTV | |
| | NATKLTVNLT NDRYLATLTV RNLVGKSDAA VLTIPACDFQ ATHPVMDLKA FPKDNMLWVE | |
| | WTTPRESVKK YILEWCVLSD KAPCITDWQQ EDGTVHRTYL RGNLAESKCY LITVTPVYAD | |
| | GPGSPESIKA YLKQAPPSKG PTVRTKKVGK NEAVLEWDQL PVDVQNGFIR NYTIFYRTII | |
| | GNETAVNVDS SHTEYTLSSL TSDTLYMVRM AAYTDEGGKD GPEFTFTTPK FAQGEIEEPR | |
| | GPTIKPCPPC KCPAPNLLGG PSVFIFPPKI KDVLMISLSP IVTCVVVDVS EDDPDVQISW | |
| | FVNNVEVHTA QTQTHREDYN STLRVVSALP IQHQDWMSGK EFKCKVNNKD LPAPIERTIS | |
| | KPKGSVRAPQ VYVLPPPEEE MTKKQVTLTC MVTDEMPEDI YVEWTNNGKT ELNYKNTEPV | |
| | LDSDGSYFMY SKLRVEKKNW VERNSYSCSV VHEGLHNHHT TKSFSRTPGK | |
| 191. | ELLDPCGYIS PESPVVQLHS NFTAVCVLKE KCMDYFHVNA NYIVWKTNHF TIPKEQYTII | AA amino |
| | NRTASSVTFT DIASLNIQLT CNILTFGQLE QNVYGITIIS GLPPEKPKNL SCIVNEGKKM | acid |
| | RCEWDGGRET HLETNFTLKS EWATHKFADC KAKRDTPTSC TVDYSTVYFV NIEVWVEAEN | sequence |
| | ALGKVTSDHI NFDPVYKVKP NPPHNLSVIN SEELSSILKL TWTNPSIKSV IILKYNIQYR | |
| | TKDASTWSQI PPEDTASTRS SFTVQDLKPF TEYVFRIRCM KEDGKGYWSD WSEEASGITY | |
| | EDRPSKAPSF WYKIDPSHTQ GYRTVQLVWK TLPPFEANGK ILDYEVTLTR WKSHLQNYTV | |
| | NATKLTVNLT NDRYLATLTV RNLVGKSDAA VLTIPACDFQ ATHPVMDLKA FPKDNMLWVE | |
| | WTTPRESVKK YILEWCVLSD KAPCITDWQQ EDGTVHRTYL RGNLAESKCY LITVTPVYAD | |
| | GPGSPESIKA YLKQAPPSKG PTVRTKKVGK NEAVLEWDQL PVDVQNGFIR NYTIFYRTII | |
| | GNETAVNVDS SHTEYTLSSL TSDTLYMVRM AAYTDEGGKD GPEFTFTTPK FAQGEIEEQK | |
| | LISEEDLGGE QKLISEEDLH HHHHH | |
| 192. | LPPEKPKNLS CIVNEGKKMR CEWDGGRETH LETNFTLKSE WATHKFADCK AKRDTPTSCT | AA amino |
| | VDYSTVYFVN IEVWVEAENA LGKVTSDHIN FDPVYKVKPN PPHNLSVINS EELSSILKLT | acid |
| | WTNPSIKSVI ILKYNIQYRT KDASTWSQIP PEDTASTRSS FTVQDLKPFT EYVFRIRCMK | sequence |
| | EDGKGYWSDW SEEASGITYE DRPSKAPSFW YKIDPSHTQG YRTVQLVWKT LPPFEANGKI | |
| | LDYEVTLTRW KSHLQNYTVN ATKLTVNLTN DRYLATLTVR NLVGKSDAAV LTIPACDFQA | |
| | THPVMDLKAF PKDNMLWVEW TTPRESVKKY ILEWCVLSDK APCITDWQQE DGTVHRTYLR | |
| | GNLAESKCYL ITVTPVYADG PGSPESIKAY LKQAPPSKGP TVRTKKVGKN EAVLEWDQLP | |
| | VDVQNGFIRN YTIFYRTIIG NETAVNVDSS HTEYTLSSLT SDTLYMVRMA AYTDEGGKDG | |
| | PEFTFTTPKF AQGEIEEQKL ISEEDLGGEQ KLISEEDLHH HHHH | |
| 193. | APSFWYKIDP SHTQGYRTVQ LVWKTLPPFE ANGKILDYEV TLTRWKSHLQ NYTVNATKLT | AA amino |
| | VNLTNDRYLA TLTVRNLVGK SDAAVLTIPA CDFQATHPVM DLKAFPKDNM LWVEWTTPRE | acid |
| | SVKKYILEWC VLSDKAPCIT DWQQEDGTVH RTYLRGNLAE SKCYLITVTP VYADGPGSPE | sequence |
| | SIKAYLKQAP PSKGPTVRTK KVGKNEAVLE WDQLPVDVQN GFIRNYTIFY RTIIGNETAV | |
| | NVDSSHTEYT LSSLTSDTLY MVRMAAYTDE GGKDGPEFTF TTPKFAQGEI EEQKLISEED | |
| | LGGEQKLISE EDLHHHHHH | |
| 194. | ELLDPCGYIS PESPVVQLHS NFTAVCVLKE KCMDYFHVNA NYIVWKINHF TIPKEQYTII | AA amino |
| | NRTASSVTFT DISSLNIQLT CNILTFGQLE QNVYGITIIS GLPPEKPKNL SCIVNEGKKM | acid |
| | RCEWNRGRET HLETNFTLKS EWATHKFADC KAKRDTPTSC TVDYSTVYFV NIEVWVEAEN | sequence |
| | ALGKVTSDHI NFDPVYKVKP NPPHNLSVIN SEELSSILKL TWTNPSIKSV IRLKYNIQYR | |
| | TKDASTWSQI PPEDTASTRS SFTVQDLKPF TEYVFRICCM KEDGKGYWSD WSEEANGITY | |
| | EDRPSKAPSF WYKIDPSHAQ GYRTVQLMWK TLPPFEANGK ILDYEVTLTR WKSHLQNYTV | |
| | NDTKLTVNLT NDRYVATLTA RNLVGKSDAA VLTIPACDFQ ATHPVMDLKA FPKDNMLWVE | |
| | WTTPRESVKK YILEWCVLSD KAPCIADWQQ EDGTVHRTHL RGNLAESKCY LITVTPVYAD | |
| | GPGSPESIKA YLKQAPPSKG PTVRTKKVGK NEAVLEWDQL PVDVQNGFIR NYTIFYRTII | |
| | GNETAVNVDS SHTEYTLSSL TSDTLYMVRM AAYTDEGGKD GPEFTFTTPK FAQGEIEEQK | |
| | LISEEDLGGE QKLISEEDLH HHHHH | |
| 195. | QLVEPCGYIY PEFPVVQRGS NFTATCVLKE KCLQVYSVNA TYIVWKTNHV AVPKEQVTVI | AA amino |
| | NRTASSVTFT DVVFQNVQLT CNILSFGQIE QNVYGITILS GYPPDIPTNL SCIVNEGKNM | acid |
| | LCQWDPGRET YLETNYTLKS EWATEKFPDC RTKHGTSSCM MGYTPIYFVN IEVWVEAENA | sequence |
| | LGNVSSEPIN FDPVDKVKPS PPHNLSVTNS EELSSILKLA WVNSGLDSIL RLKSDIQYRT | |
| | KDASTWIQVP LEDTVSPRTS FTVQDLKPFT EYVFRIRSIK ENGKGYWSDW SEEASGTTYE | |
| | DRPSKAPSFW YKVNANHPQE YRSARLIWKT LPLSEANGKI LDYEVVLTQS KSVSQTYTVN | |
| | GTELIVNLTN NRYVASLAAR NVVGKSPATV LTIPGSHFKA SHPVVDLKAF PKDNLLWVEW | |
| | TPPSKPVNKY ILEWCVLSEN SPCIPDWQQE DGTVNRTHLR GSLLESKCYL ITVTPVFPGG | |
| | PGSPESMKAY LKQAAPSKGP TVRTKKVGKN EAVLEWDHLP VDVQNGFIRN YSISYRTSVG | |
| | KEMVVRVDSS HTEYTLSSLS SDTLYMVHMA AYTEEGGKDG PEFTFTTLKF AQGEIEEQKL | |
| | ISEEDLGGEQ KLISEEDLHH HHHH | |
| 196. | QLLEPCGYIY PEFPVVQRGS NFTAICVLKE ACLQHYYVNA SYIVWKTNHA AVPREQVTVI | AA amino |
| | NRTTSSVTFT DVVLPSVQLT CNILSFGQIE QNVYGVTMLS GFPPDKPTNL TCIVNEGKNM | acid |
| | LCQWDPGRET YLETNYTLKS EWATEKFPDC QSKHGTSCMV SYMPTYYVNI EVWVEAENAL | sequence |
| | GKVSSESINF DPVDKVKPTP PYNLSVINSE ELSSILKLSW VSSGLGGLLD LKSDIQYRTK | |
| | DASTWIQVPL EDTMSPRTSF TVQDLKPFTE YVFRIRSIKD SGKGYWSDWS EEASGTTYED | |
| | RPSRPPSFWY KTNPSHGQEY RSVRLIWKAL PLSEANGKIL DYEVILTQSK SVSQTYTVTG | |
| | TELTVNLTND RYVASLAARN KVGKSAAAVL TIPSPHVTAA YSVVNLKAFP KDNLLWVEWT | |
| | PPPKPVSKYI LEWCVLSENA PCVEDWQQED ATVNRTHLRG RLLESKCYQI TVTPVFATGP | |
| | GGSESLKAYL KQAAPARGPT VRTKKVGKNE AVLAWDQIPV DDQNGFIRNY SISYRTSVGK | |
| | EMVVHVDSSH TEYTLSSLSS DTLYMVRMAA YTDEGGKDGP EFTFTTPKFA QGEIEEQKLI | |
| | SEEDLGGEQK LISEEDLHHH HHH | |
| 197. | ELLDPCGYIS PESPVVQLHS NFTAVCVLKE KCMDYFHVNA NYIVWKINHF TIPKEQYTII | AA amino |
| | NRTASSVTFT DIASLNIQLT CNILTFGQLE QNVYGITIIS GLPPEKPKNL SCIVNEGKKM | acid |
| | RCEWDGGRET HLETNFTLKS EWATHKFADC KAKRDTPTSC TVDYSTVYFV NIEVWVEAEN | sequence |
| | ALGKVTSDHI NFDPVYKVKP NPPHNLSVIN SEELSSILKL TWTNPSIKSV IILKYNIQYR | |
| | TKDASTWSQI PPEDTASTRS SFTVQDLKPF TEYVFRIRCM KEDGKGYWSD WSEEASGITY | |
| | EDRPSKAPSF WYKIDPSHTQ GYRTVQLVWK TLPPFEANGK ILDYEVTLTR WKSHLQNYTV | |
| | NATKLTVNLT NDRYLATLTV RNLVGKSDAA VLTIPACDFQ ATHPVMDLKA FPKDNMLWVE | |
| | WTTPRESVKK YILEWCVLSD KAPCITDWQQ EDGTVHRTYL RGNLAESKCY LITVTPVYAD | |
| | GPGSPESIKA YLKQAPPSKG PTVRTKKVGK NEAVLEWDQL PVDVQNGFIR NYTIFYRTII | |
| | GNETAVNVDS SHTEYTLSSL TSDTLYMVRM AAYTDEGGKD GPEFTFTTPK FAQGEIEDKT | |
| | HTCPPCPAPE LLGGPSVFLF PPKPKDTLMI SRTPEVTCVV VDVSHEDPEV KFNWYVDGVE | |
| | VHNAKTKPRE EQYNSTYRVV SVLTVLHQDW LNGKEYKCKV SNKALPAPIE KTISKAKGQP | |
| | REPQVYTLPP SRDELTKNQV SLTCLVKGFY PSDIAVEWES NGQPENNYKT TPPVLDSDGS | |
| | FFLYSKLTVD KSRWQQGNVF SCSVMHEALH NHYTQKSLSL SPGK | |
| 198. | QRHSPQEAPH VQYERLGSDV TLPCGTANWD AAVTWRVNGT DLAPDLLNGS QLVLHGLELG | AA amino |
| | HSGLYACFHR DSWHLRHQVL LHVGLPPREP VLSCRSNTYP KGFYCSWHLP TPTYIPNTFN | acid |
| | VTVLHGSKIM VCEKDPALKN RCHIRYMHLF STIKYKVSIS VSNALGHNAT AITFDEFTIV | sequence |
| | KPDPPENVVA RPVPSNPRRL EVTWQTPSTW PDPESFPLKF FLRYRPLILD QWQHVELSDG | |
| | TAHTITDAYA GKEYIIQVAA KDNEIGTWSD WSVAAHATPW TEEPRHLTTE AQAAETTTST | |
| | TSSLAPPPTT KICDPGELGS EQKLISEEDL GGEQKLISEE DLHHHHHH | |

1. An isolated bispecific antibody or antigen binding fragment thereof comprising:
(a) a first immunoglobulin heavy chain variable region (HCVR) and immunoglobulin light chain variable region (LCVR) that binds human GP130 (D1); and
(b) a second immunoglobulin heavy chain variable region (HCVR) and immunoglobulin light chain variable region (LCVR) that binds human leptin receptor (LEPR) (D2).

2. The isolated bispecific antibody or antigen-binding fragment thereof of claim 1, wherein D1 and/or D2 comprises an immunoglobulin variable domain.

3. The isolated bispecific antibody or antigen-binding fragment thereof of claim 1, wherein D1 and/or D2 comprises an immunoglobulin heavy chain variable region (HCVR) and an immunoglobulin light chain variable region (LCVR).

4. The isolated bispecific antibody or antigen-binding fragment thereof of claim 1, further comprising a multimerizing component that connects D1 with D2.

5. The isolated bispecific antibody or antigen-binding fragment thereof of claim 4, wherein the multimerizing component comprises an Fc portion of an immunoglobulin.

6. The isolated bispecific antibody or antigen binding fragment thereof of claim 1, wherein D1 is derived from an anti-GP130 antibody that exhibits one or more properties selected from the group consisting of:
(i) binds monomeric human GP130 at 25° C. with a K D of less than about 50 nM as measured by surface plasmon resonance;
(ii) binds monkey GP130 but does not substantially bind rat or mouse GP130 as determined by surface plasmon resonance;
(iii) does not inhibit GP130 ligand-mediated signaling in a cell based GP130 signaling assay; and
(iv) does not activate GP130 signaling in the absence of a GP130 ligand.

7. The isolated bispecific antibody or antigen binding fragment thereof of claim 1, wherein D2 is derived from an anti-LEPR antibody that exhibits one or more properties selected from the group consisting of:
(i) binds monomeric human LEPR at 25° C. with a K D of less than about 110 nM as measured by surface plasmon resonance; and
(ii) potentiates leptin-mediated signaling in vitro.

8. The isolated bispecific antibody or antigen binding fragment thereof of claim 1, wherein the bispecific antibody or antigen binding fragment thereof exhibits one or more properties selected from the group consisting of:
(i) binds monomeric human LEPR at 25° C. with a K D of less than about 110 nM as measured by surface plasmon resonance;
(ii) binds monomeric human LEPR at 25° C. with a t ½ of greater than about 3 minutes as measured by surface plasmon resonance;
(iii) binds monomeric human GP130 at 25° C. with a K D of less than about 150 nM as measured by surface plasmon resonance;
(iv) binds monomeric human GP130 at 25° C. with a t ½ of greater than about 2.5 minutes as measured by surface plasmon resonance;
(v) binds monkey LEPR;
(vi) binds monkey GP130;
(vii) binds cells expressing human LEPR in the presence or absence of leptin as measured by FACS;
(viii) activates GP130 in a cell-based assay with a potency that is at least 20% or greater than the activation mediated by human oncostatin M under the same or similar experimental assay conditions; and
(ix) causes a reduction in body weight when administered in a therapeutically effective dose to an animal.

9. The isolated bispecific antibody or antigen binding fragment thereof of claim 1, wherein D1 comprises three heavy chain complementarity determining regions (D1-HCDR1, D1-HCDR2 and D1-HCDR3) from a heavy chain variable region (D1-HCVR) comprising the amino acid sequence of SEQ ID NO: 154 and three light chain complementarity determining regions (D1-LCDR1, D1-LCDR2 and D1-LCDR3) from a light chain variable region (D1-LCVR) comprising the amino acid sequence of SEQ ID NO: 10.

10. The isolated bispecific antibody or antigen binding fragment thereof of claim 9, wherein D1-HCDR1 comprises the amino acid sequence of SEQ ID NO:
156; D1-HCDR2 comprises the amino acid sequence of SEQ ID NO: 158; D1-HCDR3 comprises the amino acid sequence of SEQ ID NO: 160; D1-LCDR1 comprises the amino acid sequence of SEQ ID NO: 12; D1-LCDR2 comprises the amino acid sequence of SEQ ID NO: 14; and D1-LCDR3 comprises the amino acid sequence of SEQ ID NO: 16.

11. The isolated bispecific antibody or antigen binding fragment thereof of claim 10, wherein D1-HCVR comprises the amino acid sequence of SEQ ID NO: 154; and D1-LCVR comprises the amino acid sequence of SEQ ID NO:10.

12. The isolated bispecific antibody or antigen binding fragment thereof of claim 1, wherein D2 comprises three heavy chain complementarity determining regions (D2-HCDR1, D2-HCDR2 and D2-HCDR3) from a heavy chain variable region (D2-HCVR) comprising the amino acid sequence selected from the group consisting of SEQ ID NOs: 2, 162, 170 and 178; and three light chain complementarity determining regions (D2-LCDR1, D2-LCDR2 and D2-LCDR3) from a light chain variable region (D2-LCVR) comprising the amino acid sequence of SEQ ID NO: 10.

13. The isolated bispecific antibody or antigen binding fragment thereof of claim 12, wherein D2-HCVR comprises D2-HCDR1, D2-HCDR2, D2-HCDR3 amino acid sequences selected from the group consisting of SEQ ID NOs: 4, 6, 8, respectively; SEQ ID NOs: 164,166, 168, respectively; SEQ ID NOs: 172, 174, 176, respectively, and SEQ ID NOs: 180, 182, 184, respectively; and wherein the D2-LCVR comprises D2-LCDR1, D2-LCDR2, D2-LCDR3 amino acid sequences of SEQ ID NOs: 12, 14, 16, respectively.

14. The isolated bispecific antibody or antigen binding fragment thereof of claim 13, wherein D2-HCVR comprises an amino acid sequence selected from the group consisting of SEQ ID NOs: 2, 162, 170 and 178; and D1-LCVR comprises the amino acid sequence of SEQ ID NO: 10.

15. The isolated bispecific antibody or antigen-binding fragment thereof of claim 1, wherein D2 is derived from an anti-LEPR antibody that potentiates leptin-mediated signaling in vitro through the LEPR-b isoform.

16. The isolated bispecific antibody or antigen-binding fragment thereof of claim 1, wherein D2 is derived from an anti-LEPR antibody that does not activate leptin-mediated signaling in vitro through the LEPR-a isoform.

17. The isolated bispecific antibody or antigen-binding fragment thereof of claim 1, wherein the bispecific antibody or antigen binding fragment thereof activates signaling through the LEPR-b isoform and does not substantially activate leptin-mediated signaling through the LEPR-a isoform.

18. A pharmaceutical composition comprising the isolated bispecific antibody or antigen-binding fragment thereof of claim 1, and a pharmaceutically acceptable carrier or diluent.

19. A vessel or injection device comprising the isolated bispecific antibody or antigen-binding fragment thereof of claim 1.

20. An isolated nucleic acid encoding the immunoglobulin chains of the bispecific antibody or antigen-binding fragment thereof of claim 1.

21. An isolated vector comprising the nucleic acid of claim 20.

22. An isolated host cell comprising the vector of claim 21.

23. The host cell of claim 22, which is a Chinese hamster ovary cell.

24. A method for treating a disease or condition, in a subject, associated with or caused by leptin deficiency or leptin resistance, the method comprising administering the pharmaceutical composition of claim 18, to a subject in need thereof.

25. The method of claim 24, wherein the disease or condition associated with or caused by leptin deficiency or leptin resistance is selected from the group consisting of lipodystrophies, obesity, metabolic syndrome, diet-induced food craving, functional hypothalamic amenorrhea, type 1 diabetes, type 2 diabetes, insulin resistance, severe insulin resistance due to mutation in insulin receptor, Alzheimer's disease, leptin deficiency, leptin resistance, Leprechaunism/Donohue syndrome, and Rabson-Mendenhall syndrome.

26. A method for treating a lipodystrophy condition in a subject, the method comprising administering the pharmaceutical composition of claim 18, to a subject in need thereof, wherein the lipodystrophy condition is selected from the group consisting of congenital generalized lipodystrophy, acquired generalized lipodystrophy, familial partial lipodystrophy, acquired partial lipodystrophy, centrifugal abdominal lipodystrophy, lipoatrophia annularis, localized lipodystrophy, and HIV-associated lipodystrophy.

27. The method of claim 24, further comprising administering a second therapeutic agent to the subject, wherein the second therapeutic agent is selected from the group consisting of a recombinant human leptin, a PCSK9 inhibitor, a statin, ezetimibe, insulin, an insulin variant, an insulin secretagogue, metformin, a sulfonylurea, a sodium glucose cotransporter 2 (SGLT2) Inhibitor, a GLP-1 agonist/analogue, a glucagon (GCG) inhibitor, a glucagon receptor (GCGR) inhibitor, an angiopoietin-like protein (ANGPTL) inhibitor, Phentermine, Orlistat, Topiramate, Bupropion, Topiramate/Phentermine, Bupropion/Naltrexone, Bupropion/Zonisamide, Pramlintide/Metrelepin, Lorcaserin, Cetilistat, Tesofensine, and Velneperit.

28. A method for making the bispecific antibody or antigen-binding fragment thereof of claim 1, comprising:
(a) introducing one or more polynucleotides encoding an immunoglobulin chain of said antibody or antigen-binding fragment thereof into a host cell;
(b) culturing the host cell under conditions favorable to expression of the polynucleotide; and
(c) optionally, isolating the antibody or antigen-binding fragment thereof from the host cell and/or medium in which the host cell is grown.

29. A bispecific antigen-binding molecule or immunoglobulin chain which is a product of the method of claim 28.

30. A method for administering the isolated bispecific antibody or antigen-binding fragment thereof of claim 1, to a subject comprising injecting said antibody or antigen-binding fragment thereof into the body of the subject.

31. The method of claim 30, wherein said antibody or antigen-binding fragment thereof is injected intravenously, intramuscularly or subcutaneously.

32. A method for treating a disease or condition, in a subject in need thereof, associated with or caused by leptin deficiency or leptin resistance, the method comprising administering a therapeutically effective dose of the isolated bispecific antibody or antigen binding fragment thereof comprising:
(a) a first immunoglobulin heavy chain variable region (HCVR) and immunoglobulin light chain variable region (LCVR) that binds human GP130 (D1); and
(b) a second immunoglobulin heavy chain variable region (HCVR) and immunoglobulin light chain variable region (LCVR) that binds human leptin receptor (LEPR) (D2);
which, when contacted by a cell that expresses human LEPR having a Y1141F mutation and human gp130 on the surface of the cell, in the presence of human leptin, causes heterodimerization of the LEPR and gp130 and transcription of STAT3 in the cell;
wherein said transcription of STAT3 is not activated in said cell when contacted by:
a monospecific antibody that binds gp130 and comprises said immunoglobulin heavy chain variable region (HCVR) and said immunoglobulin light chain variable region (LCVR) (D1); and
a monospecific antibody that binds LEPR and comprises said immunoglobulin heavy chain variable region (HCVR) and said immunoglobulin light chain variable region (LCVR) (D2), but not contacted by said bispecific antibody or antigen binding fragment thereof; to the subject.

33. The method of claim 32, wherein the disease or condition associated with or caused by leptin deficiency or leptin resistance is selected from the group consisting of obesity, metabolic syndrome, diet-induced food craving, functional hypothalamic amenorrhea, type 1 diabetes, type 2 diabetes, insulin resistance, severe insulin resistance due to mutation in insulin receptor, Alzheimer's disease, leptin deficiency, leptin resistance, Leprechaunism/Donohue syndrome, and Rabson-Mendenhall syndrome.

34. The method of claim 32, further comprising administering a second therapeutic agent to the subject.

35. The method of claim 32, further comprising administering a second therapeutic agent to the subject, wherein the second therapeutic agent is selected from the group consisting of a recombinant human leptin, a PCSK9 inhibitor, a statin, ezetimibe, insulin, an insulin variant, an insulin secretagogue, metformin, a sulfonylurea, a sodium glucose cotransporter 2 (SGLT2) Inhibitor, a GLP-1 agonist/analogue, a glucagon (GCG) inhibitor, a glucagon receptor (GCGR) inhibitor, an angiopoietin-like protein (ANGPTL) inhibitor, Phentermine, Orlistat, Topiramate, Bupropion, Topiramate/Phentermine, Bupropion/Naltrexone, Bupropion/Zonisamide, Pramlintide/Metrelepin, Lorcaserin, Cetilistat, Tesofensine, and Velneperit.

36. The method of claim 32, wherein said antibody or antigen-binding fragment thereof is administered by injecting intravenously, intramuscularly or subcutaneously.
